(19) United States
(12) Patent Application Publication
(10) Pub. No.: US 2020/0084186 A1
(43) Pub. Date: Mar. 12, 2020

(54) Encrypted Messaging System

(71) Applicant: Medroster.com Corporation, Santa Monica, CA (US)

(72) Inventors: Changgao Yang, Signal Hill, CA (US); Keith Parks, Long Beach, CA (US); Christopher Scott Whitman, Los Angeles, CA (US); Martin Boßlet, Bechhofen (DE)

(21) Appl. No.: 16/538,729

(22) Filed: Aug. 12, 2019

Related U.S. Application Data
(60) Provisional application No. 62/717,691, filed on Aug. 10, 2018.

(51) Int. Cl. H04L 29/06; H04L 9/08; G16H 40/67; G16H 40/20; H04L 12/58

(52) U.S. Cl. CPC H04L 63/0435; H04L 9/083; H04L 63/08; H04L 2463/121; G16H 40/20; H04L 67/42; H04L 51/04; G16H 40/67

(57) ABSTRACT

An encrypted messaging system allows secured communication for the medical industry. The encrypted messaging system may be designed to observe strict confidentiality requirements for various use cases required in the medical industry, such as the confidentiality requirements required by HIPAA. For example, the encrypted messaging system may include features that are specifically designed for interoffice, intraoffice, or patient communications, while maintaining privacy of the information being transmitted within the encrypted messaging system.

A portion of the disclosure of this patent document contains material which is subject to copyright protection. The copyright owner has no objection to the facsimile reproduction by anyone of the patent document or the patent disclosure, as it appears in the U.S. Patent and Trademark Office patent file or records, but otherwise reserves all copyright rights whatsoever.

### CROSS REFERENCE TO RELATED APPLICATIONS

This patent application claims the benefit of U.S. patent application 62/717,691, filed Aug. 10, 2018, which is incorporated by reference along with all other references cited in this application.

### BACKGROUND OF THE INVENTION

The present invention relates to a messaging system and, more specifically, an encrypted messaging system that maintains the confidentiality of information shared in the encrypted messaging system.

The Internet has improved communication in many industries. Messages and other information can be transmitted over the Internet and accessed across a variety of devices, often nearly simultaneously to when the message is sent.

However, some industries have lagged behind. For example, much of the communication in the medical industry relies on using a fax machine, which is inefficient, costly, and produces poor image quality. This makes it difficult for medical practitioners to communicate with patients, other doctors, or within their own office.

One of the problems to overcome is that the medical industry is required to observe strict confidentiality of patient information. Some of these requirements are laid out by Health Insurance Portability and Accountability Act of 1996 (HIPAA) which is legislation passed in the United States that provides data privacy and security provisions for safeguarding patient information. This patient information may include sensitive identifying information of patients such as social security card numbers, addresses, or other information. Also, because a medical professional has access to test results and diagnoses for diseases and other ailments, inadvertent exposure of patient information may result in exposing a patient to disrepute. Failure to abide by the strict confidentiality standards may erode patient confidence in their medical provider or even result in legal liability for violation of HIPAA.

Therefore, there is a need for an improved communication method for the medical industry.

### BRIEF SUMMARY OF THE INVENTION

An encrypted messaging system allows secured communication for the medical industry. The encrypted messaging system may be designed to observe strict confidentiality requirements for various use cases required in the medical industry, such as the confidentiality requirements required by HIPAA. For example, the encrypted messaging system may include features that are specifically designed for interoffice, intraoffice, or patient communications, while maintaining privacy of the information being transmitted within the encrypted messaging system.

In an implementation, the encrypted messaging system is the Medroster software product provided by Medroster.com Corporation. The Medroster software product is a HIPAA-secure communications platform for the medical community. The Medroster software product makes communication between doctors, their staff and patients safer and more efficient by simulating how these users would communicate in real life. Medroster is cross-platform and works across desktop, iOS, Android, and tablet.

HIPAA is implemented by the HIPAA Administrative Simplification Regulations specified by the Code of Federal Regulations in 45 CFR 160, 162, and 164, which is hereby incorporated by reference. Specifically, section 164.312(e)(ii) discusses technical security measures to guard against unauthorized access to electronic protected health information (or EPHI) that is being transmitted over an electronic communications network. The US Health and Human Services has also provided additional guidelines in the Federal Register and other materials regarding security standards required by HIPAA which are also incorporated by reference to the filing date of this patent application.

In an implementation, the encrypted messaging system includes a method of fetching, from a key server, a first encryption key for a first conversation from a first client device intended for a medical space that corresponds to a second client device. The medical space may be part of a medical office, business entity, a HIPAA compliant unit, or, in larger medical establishments, a department or any division used by the medical industry. The first conversation may be a message from a patient to a medical office or vice versa.

The medical office may include multiple medical spaces, such as sections or departments within a place or a doctor's office. Some examples of medical spaces include front office, back office, reception, billing, doctor, other staff, or any other space in a medical office. In an implementation, for larger institutions such as a hospital, the medical place may be defined as a department in a hospital, for manageability and ease of use of the encrypted messaging system.

The medical space may correspond a user logged onto the second client device and may be any type of device that can access the encrypted messaging system, such as a personal computer, tablet computer, smart phone, or smart device. The medical space may correspond to more than one user account. For example, the second client device may be logged in as a first user. However, the medical space may also correspond to a second user, different than the first user, which is associated with the same medical space. The second user may log onto the second client device or a different client device.

The encrypted messaging system includes encrypting the first conversation using the first encryption key to create a first encrypted conversation. The encrypted messaging system may use any suitable method of encryption, such as Advanced Encryption Standard (AES), RSA (Rivest-Shamir-Adelman), or any other encryption scheme. The encrypted messaging system includes storing a first timestamp and a first conversation identifier that uniquely identifies the first encrypted conversation with the first encryption key and transmitting the first encrypted conversation to a message server. The first timestamp may correspond to a time when the first client device has completed drafting the first conversation, when a user of the first client device indicated to send the first conversation, when the first conversation has been requested by the second client device, or any other period of time. The first conversation identifier may be a unique conversation identifier that is not reused in the encrypted messaging system to identify any other conversation in the encrypted messaging system. The conversation identifier may be a randomized conversation identifier. This means that the first conversation identifier is generated using a random number generator of the encrypted messaging system. This may prevent malicious users from easily guessing conversation identifiers for conversations in the encrypted messaging system, which may make the process of hacking or accessing the first conversation more difficult. The message server may be a different server than the key server. The message server may also be the same server as the key server, such as a server supporting features of the message and key servers on different virtualized systems or as applications executing on the same server.

The encrypted messaging system includes when exceeding an expiration period for the first conversation according to the first timestamp, causing to be deleted the first encrypted message from the message server. For example, the encrypted messaging system may include sending a message to the message server to delete the first encrypted conversation or the message server may automatically delete the first encrypted conversation without a message from another server. The expiration period may be any type of expiration period, such as a period of time (e.g., 1 day, 2 days, 3 days, 1 week, or other length of time), a number of access attempts to the first encrypted conversation, a number of successful access attempts to the first encrypted conversation, a number of clients that have accessed the first encrypted conversation (e.g., all parties to the conversation, at least one party to the conversation), or a combination of any of these.

Deleting the first encrypted message may occur before or after the first encrypted conversation has been requested by the second client device. The encrypted messaging system includes receiving from the second client device a request to retrieve the first encrypted conversation from the message server.

If the first encrypted conversation has been deleted, the encrypted messaging system includes responding to the second client device that the first encrypted message has been deleted. If the first encrypted conversation has not been deleted, the encrypted messaging system includes causing to be determined at the second client device, based on the first conversation identifier, whether the second client device has stored a copy of a first decryption key. For example, the first decryption key may be stored on the second client device in a cache or other memory store. The memory store may also be encrypted to prevent unauthorized access to the first decryption key. When the second client device has not stored a copy of the first decryption key, the encrypted messaging system includes receiving a request at the key server for the first encryption key.

In an implementation, the encrypted messaging system includes decryption keys that are conversation specific. This means that there may be one or more messages in the first encrypted conversation, such as a back and forth between users to coordinate an appointment time. The first decryption key may decrypt all the messages associated with the first encrypted conversation, without needing an additional decryption key for each specific message in the conversation. Further, the first decryption key may be unable to decrypt other conversations in the encrypted messaging system. For example, a second conversation in the encrypted messaging system may be unable to be decrypted using the first decryption key, even if the parties included in the second encrypted conversation are the same as the parties in the first encrypted conversation.

In an implementation, the encrypted messaging system includes ephemeral conversations. This means that, although a conversation must be downloaded onto client devices for viewing, once the conversation has been closed, the conversation is deleted from client devices. Deleting from client devices may occur when a connection to the encrypted messaging system is terminated at the client device, when the encrypted messaging system application is terminated at the client device, upon a timeout period for the client device (e.g., connected to the encrypted messaging system's servers for a period of time, activity from the client device has not occurred for a period of time), or other. This allows the encrypted messaging system to maintain confidentiality of conversations in the encrypted messaging system, by restricting the number of computers which store copies of conversations. In an implementation, client devices may retain encrypted conversations in memory of the client devices. This allows flexibility for storage of conversations and conserves bandwidth by preventing duplicative downloading of conversations.

In an implementation, the encrypted messaging system includes a method for a secured messaging system including: receiving from a patient user's device a request to secure a first message to be sent to a first functional unit of a medical office. A functional unit may be a grouping of one or more users of the encrypted messaging system, that share responsibilities in the encrypted messaging system. A single user may be part of more than one functional unit. For example, the patient user may be attempting to reach a doctor, front office, or other functional unit of the system. The encrypted messaging system may include in response to the patient user's request, selecting a first encryption key and creating, based on the first message and the first encryption key, a secured first message. The encrypted messaging system may include causing storing the secured first message and a unique identifier for the secured first message. The encrypted messaging system determines which users of the encrypted messaging system the first message was intended for. Since there may be multiple medical offices using the encrypted messaging system simultaneously, the encrypted messaging system may determine users associated with the medical office. Then, based on the users associated with the medical office, the encrypted messaging system determines first and second users of the secured messaging system associated with the first functional unit and the medical office and transmits the secured first message to these users. The encrypted messaging system may include causing to be determined, based on the unique identifier of the secured first message, that a first decryption key is stored on the first user's device before transmitting the first secured message; and decrypting the first secured message on the first user's device. Other implementations of the encrypted messaging system may check whether the first decryption key is stored on the first user's device before the first user's request to open the message, in response to the first and second users being determined, or at other times.

In an implementation, the encrypted messaging system includes determining that the first decryption key is not stored on the second user's device. For example, the encrypted messaging system includes causing to be determined, based on the unique identifier of the secured first message, that the first decryption key is not stored on the second user's device before transmitting the first secured message, transmitting, based on the second user's device not storing the first decryption key, the first decryption key to the second user's device; and decrypting the first secured message on the second user's device. The encrypted messaging system may include where transmitting the first decryption key and transmitting the secured first message occurs during different transmissions.

In an implementation, the first and second users may be different roles in the encrypted messaging system. For example, the first user includes a staff role user and the second user includes a doctor role user.

In an implementation, the encrypted messaging system includes allowing responses from a medical office to the patient user. The encrypted messaging system includes receiving from the first user's device a request to secure a second message to be sent to the patient user; in response to the first user's request, selecting a second encryption key; creating, based on the second message and the second encryption key, a secured second message; causing storing the secured second message and a unique identifier for the secured second message; transmitting the secured second message to the patient user's device; causing to be determined, based on the unique identifier of the secured second message, that a second decryption key is stored on the patient user's device before transmitting the second secured message; and decrypting the second secured message on the patient user's device. The encrypted messaging system may include an indication that the first user is responding to the patient user on behalf of the second user. For example, the second secured message may include a badge or a icon that designates the second secured message as being sent by a staff member working with a doctor to respond to the second secured message.

In an implementation, the patient user opens the second secured message from the first user. The encrypted messaging system may include receiving from the first user's device a request to secure a second message to be sent to the patient user; in response to the first user's request, selecting a second encryption key; creating, based on the second message and the second encryption key, a secured second message; causing storing the secured second message and a unique identifier for the secured second message; transmitting the secured second message to the patient user's device; causing transmitting, based on the unique identifier of the secured second message and that the second decryption key is not stored on the patient user's device before transmitting the second secured message, the second decryption key to the patient user's device; and decrypting the second secured message on the patient user's device. The encrypted messaging system does not check whether the patient user belongs to a medical office or a functional unit.

Other objects, features, and advantages of the present invention may become apparent upon consideration of the following detailed description and the accompanying drawings, in which like reference designations represent like features throughout the figures.

### BRIEF DESCRIPTION OF THE DRAWINGS

FIG. 1 shows a distributed computer network.

FIG. 2 shows a computer system that can be used in an encrypted messaging system.

FIG. 3 shows a system block diagram of the computer system.

FIGS. 4-5 show examples of mobile devices.

FIG. 6 shows a system block diagram of a mobile device.

FIG. 7 shows a system diagram of the encrypted messaging system.

FIG. 8 shows a flow of encrypting messages in the encrypted messaging system.

FIG. 9 shows a flow for messaging functional spaces in the encrypted messaging system.

FIG. 10 shows a chat encryption overview for the encrypted messaging system.

FIG. 11 shows characteristics of the encrypted messaging system when sending an encrypted message in the encrypted messaging system.

FIG. 12 shows characteristics of the encrypted messaging system when sending an encrypted message in the encrypted messaging system.

FIG. 13 shows characteristics of the encrypted messaging system when sending an encrypted message in the encrypted messaging system.

FIGS. 14-39D show screens of messaging features in the encrypted messaging system.

FIGS. 40-60 show screen guiding through a user during a signup process in the encrypted messaging system.

FIGS. 61-73 shows screens for an onboarding tutorial in the encrypted messaging system.

FIG. 74 shows a screen for a user profile.

FIG. 75 shows a screen for a place profile.

FIG. 76 shows a screen for a place profile referral.

FIG. 77-83 shows screens for a user dropdown specification.

FIGS. 84-85 show screens for a transferred user interface.

FIGS. 86-89 show screens for a people/places search feature.

FIGS. 90-94 shows screens for a reminders feature.

FIGS. 95-98B show screens for a chat feature in a web messenger application.

FIGS. 99-102 show screens for a verification feature.

FIG. 103 shows a screen with the online/offline status capability.

FIGS. 104-111 show a series of screens on how to add places for an encrypted messaging system.

FIG. 112 shows a screen of a search results pages with points of reference.

FIG. 113 shows a screen of an invite form.

FIG. 114 shows a screen of an invite form, without adding them to an existing medical practice.

### DETAILED DESCRIPTION OF THE INVENTION

An encrypted messaging system includes various features that facilities different use cases for medical industry professionals. The encrypted messaging system may be designed to protect confidential or sensitive information being stored or transmitted using the encrypted messaging system. Some examples of this information include electronic protected health information (or ePHI) protected by HIPAA, such as patient names, addresses, social security numbers, procedure codes, birth dates, and other. However, the encrypted messaging system may also protect information that is not explicitly covered by HIPAA.

FIG. 1 is a simplified block diagram of a distributed computer network 100 incorporating an embodiment of the present invention. Computer network 100 includes a number of client systems 113,116, and 119, and a server system 122 coupled to a communication network 124 via a plurality of communication links 128. Communication network 124 provides a mechanism for allowing the various components of distributed network 100 to communicate and exchange information with each other.

Communication network 124 may itself be comprised of many interconnected computer systems and communication links. Communication links 128 may be hardwire links, optical links, satellite or other wireless communications links, wave propagation links, or any other mechanisms for communication of information. Communication links 128 may be DSL, Cable, Ethernet or other hardwire links, passive or active optical links, 3G, 3.5G, 4G and other mobility, satellite or other wireless communications links, wave propagation links, or any other mechanisms for communication of information.

Various communication protocols may be used to facilitate communication between the various systems shown in FIG. 1. These communication protocols may include VLAN, MPLS, TCP/IP, Tunneling, HTTP protocols, wireless application protocol (WAP), vendor-specific protocols, customized protocols, and others. While in one embodiment, communication network 124 is the Internet, in other embodiments, communication network 124 may be any suitable communication network including a local area network (LAN), a wide area network (WAN), a wireless network, an intranet, a private network, a public network, a switched network, and combinations of these, and the like.

Distributed computer network 100 in FIG. 1 is merely illustrative of an embodiment incorporating the present invention and does not limit the scope of the invention as recited in the claims. One of ordinary skill in the art would recognize other variations, modifications, and alternatives. For example, more than one server system 122 may be connected to communication network 124. As another example, a number of client systems 113, 116, and 119 may be coupled to communication network 124 via an access provider (not shown) or via some other server system.

Client systems 113, 116, and 119 typically request information from a server system which provides the information. For this reason, server systems typically have more computing and storage capacity than client systems. However, a particular computer system may act as both as a client or a server depending on whether the computer system is requesting or providing information. Additionally, although aspects of the invention have been described using a client-server environment, it should be apparent that the invention may also be embodied in a stand-alone computer system.

Server 122 is responsible for receiving information requests from client systems 113, 116, and 119, performing processing required to satisfy the requests, and for forwarding the results corresponding to the requests back to the requesting client system. The processing required to satisfy the request may be performed by server system 122 or may alternatively be delegated to other servers connected to communication network 124.

Client systems 113, 116, and 119 enable users to access and query information stored by server system 122. In a specific embodiment, the client systems can run as a standalone application such as a desktop application or mobile smartphone or tablet application. In another embodiment, a “Web browser” application executing on a client system enables users to select, access, retrieve, or query information stored by server system 122. Examples of Web browsers include the Internet Explorer browser program provided by Microsoft Corporation, Firefox browser provided by Mozilla, Chrome browser provided by Google, Safari browser provided by Apple, and others.

In a client-server environment, some resources (e.g., files, music, video, or data) are stored at the client while others are stored or delivered from elsewhere in the network, such as a server, and accessible via the network (e.g., the Internet). Therefore, the user's data can be stored in the network or “cloud.” For example, the user can work on documents on a client device that are stored remotely on the cloud (e.g., server). Data on the client device can be synchronized with the cloud.

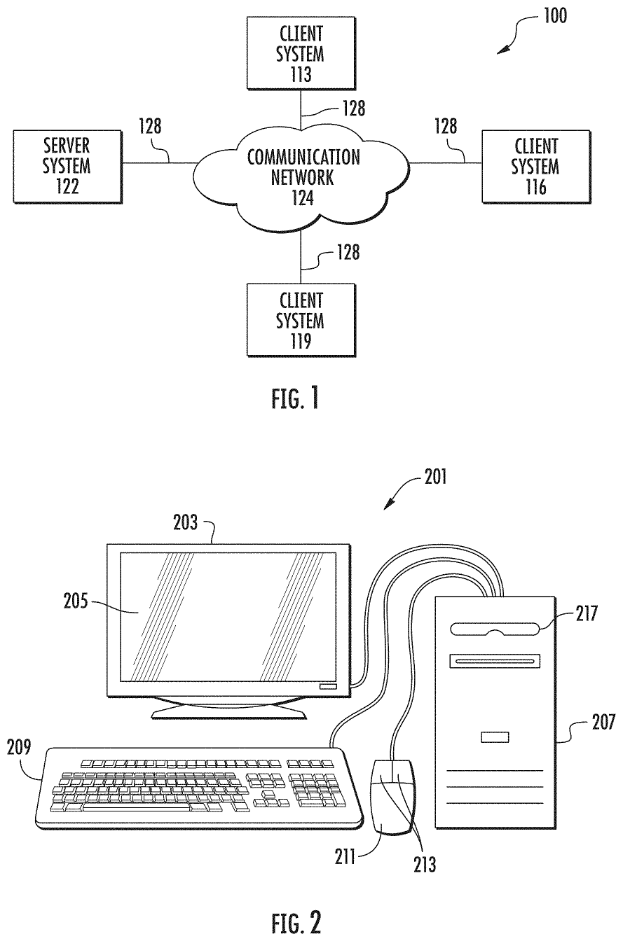

FIG. 2 shows an exemplary client or server system of the present invention. In an embodiment, a user interfaces with the system through a computer workstation system, such as shown in FIG. 2. FIG. 2 shows a computer system 201 that includes a monitor 203, screen 205, enclosure 207 (may also be referred to as a system unit, cabinet, or case), keyboard or other human input device 209, and mouse or other pointing device 211. Mouse 211 may have one or more buttons such as mouse buttons 213.

It should be understood that the present invention is not limited any computing device in a specific form factor (e.g., desktop computer form factor), but can include all types of computing devices in various form factors. A user can interface with any computing device, including smartphones, personal computers, laptops, electronic tablet devices, global positioning system (GPS) receivers, portable media players, personal digital assistants (PDAs), other network access devices, and other processing devices capable of receiving or transmitting data.

For example, in a specific implementation, the client device can be a smartphone or tablet device, such as the Apple iPhone (e.g., Apple iPhone 6), Apple iPad (e.g., Apple iPad, Apple iPad Pro, or Apple iPad mini), Apple iPod (e.g., Apple iPod Touch), Samsung Galaxy product (e.g., Galaxy S series product or Galaxy Note series product), Google Nexus and Pixel devices (e.g., Google Nexus 6, Google Nexus 7, or Google Nexus 9), and Microsoft devices (e.g., Microsoft Surface tablet). Typically, a smartphone includes a telephony portion (and associated radios) and a computer portion, which are accessible via a touch screen display.

There is nonvolatile memory to store data of the telephone portion (e.g., contacts and phone numbers) and the computer portion (e.g., application programs including a browser, pictures, games, videos, and music). The smartphone typically includes a camera (e.g., front facing camera or rear camera, or both) for taking pictures and video. For example, a smartphone or tablet can be used to take live video that can be streamed to one or more other devices.

Enclosure 207 houses familiar computer components, some of which are not shown, such as a processor, memory, mass storage devices 217, and the like. Mass storage devices 217 may include mass disk drives, floppy disks, magnetic disks, optical disks, magneto-optical disks, fixed disks, hard disks, CD-ROMs, recordable CDs, DVDs, recordable DVDs (e.g., DVD-R, DVD+R, DVD-RW, DVD+RW, HD-DVD, or Blu-ray Disc), flash and other nonvolatile solid-state storage (e.g., USB flash drive or solid state drive (SSD)), battery-backed-up volatile memory, tape storage, reader, and other similar media, and combinations of these.

A computer-implemented or computer-executable version or computer program product of the invention may be embodied using, stored on, or associated with computer-readable medium. A computer-readable medium may include any medium that participates in providing instructions to one or more processors for execution. Such a medium may take many forms including, but not limited to, nonvolatile, volatile, and transmission media. Nonvolatile media includes, for example, flash memory, or optical or magnetic disks. Volatile media includes static or dynamic memory, such as cache memory or RAM. Transmission media includes coaxial cables, copper wire, fiber optic lines, and wires arranged in a bus. Transmission media can also take the form of electromagnetic, radio frequency, acoustic, or light waves, such as those generated during radio wave and infrared data communications.

For example, a binary, machine-executable version, of the software of the present invention may be stored or reside in RAM or cache memory, or on mass storage device 217. The source code of the software of the present invention may also be stored or reside on mass storage device 217 (e.g., hard disk, magnetic disk, tape, or CD-ROM). As a further example, code of the invention may be transmitted via wires, radio waves, or through a network such as the Internet.

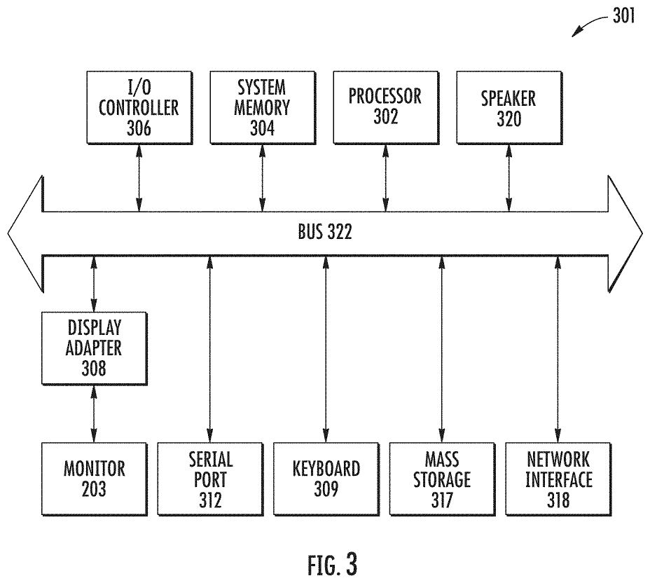

FIG. 3 shows a system block diagram of computer system 201 used to execute the software of the present invention. As in FIG. 2, computer system 201 includes monitor 203, keyboard 209, and mass storage devices 217. Computer system 201 further includes subsystems such as central processor 302, system memory 304, input/output (I/O) controller 306, display adapter 308, serial or universal serial bus (USB) port 312, network interface 318, and speaker 320. The invention may also be used with computer systems with additional or fewer subsystems. For example, a computer system could include more than one processor 302 (i.e., a multiprocessor system) or a system may include a cache memory.

Arrows such as 322 represent the system bus architecture of computer system 201. However, these arrows are illustrative of any interconnection scheme serving to link the subsystems. For example, speaker 320 could be connected to the other subsystems through a port or have an internal direct connection to central processor 302. The processor may include multiple processors or a multicore processor, which may permit parallel processing of information. Computer system 201 shown in FIG. 3 is but an example of a computer system suitable for use with the present invention. Other configurations of subsystems suitable for use with the present invention may be readily apparent to one of ordinary skill in the art.

Computer software products may be written in any of various suitable programming languages, such as C, C++, C#, Pascal, Fortran, Perl, Matlab (from MathWorks, www.mathworks.com), SAS, SPSS, JavaScript, AJAX, Java, Python, Erlang, and Ruby on Rails. The computer software product may be an independent application with data input and data display modules. Alternatively, the computer software products may be classes that may be instantiated as distributed objects. The computer software products may also be component software such as Java Beans (from Oracle Corporation) or Enterprise Java Beans (EJB from Oracle Corporation).

An operating system for the system may be one of the Microsoft Windows® family of systems (e.g., Windows 95, 98, Me, Windows NT, Windows 2000, Windows XP, Windows XP x64 Edition, Windows Vista, Windows 7, Windows 8, Windows 10, Windows CE, Windows Mobile, Windows RT), Symbian OS, Tizen, Linux, HP-UX, UNIX, Sun OS, Solaris, Mac OS X, Apple iOS, Android, Alpha OS, AIX, IRIX32, or IRIX64. Other operating systems may be used. Microsoft Windows is a trademark of Microsoft Corporation.

Any trademarks or service marks used in this patent are property of their respective owner. Any company, product, or service names in this patent are for identification purposes only. Use of these names, logos, and brands does not imply endorsement.

Furthermore, the computer may be connected to a network and may interface to other computers using this network. The network may be an intranet, internet, or the Internet, among others. The network may be a wired network (e.g., using copper), telephone network, packet network, an optical network (e.g., using optical fiber), or a wireless network, or any combination of these. For example, data and other information may be passed between the computer and components (or steps) of a system of the invention using a wireless network using a protocol such as Wi-Fi (IEEE standards 802.11, 802.11a, 802.11b, 802.11e, 802.11g, 802.11i, 802.11n, 802.11ac, and 802.11ad, just to name a few examples), near field communication (NFC), radio-frequency identification (RFID), mobile or cellular wireless (e.g., 2G, 3G, 4G, 3GPP LTE, WiMAX, LTE, LTE Advanced, Flash-OFDM, HIPERMAN, iBurst, EDGE Evolution, UMTS, UMTS-TDD, ixRDD, and EV-DO). For example, signals from a computer may be transferred, at least in part, wirelessly to components or other computers.

In an embodiment, with a Web browser executing on a computer workstation system, a user accesses a system on the World Wide Web (WWW) through a network such as the Internet. The Web browser is used to download Web pages or other content in various formats including HTML, XML, text, PDF, and postscript, and may be used to upload information to other parts of the system. The Web browser may use uniform resource identifiers (URLs) to identify resources on the Web and hypertext transfer protocol (HTTP) in transferring files on the Web.

In other implementations, the user accesses the system through either or both of native and nonnative applications. Native applications are locally installed on the particular computing system and are specific to the operating system or one or more hardware devices of that computing system, or a combination of these. These applications (which are sometimes also referred to as “apps”) can be updated (e.g., periodically) via a direct internet upgrade patching mechanism or through an applications store (e.g., Apple iTunes and App store, Google Play store, Windows Phone store, and Blackberry App World store).

The system can run in platform-independent, nonnative applications. For example, client can access the system through a Web application from one or more servers using a network connection with the server or servers and load the Web application in a Web browser. For example, a Web application can be downloaded from an application server over the Internet by a Web browser. Nonnative applications can also be obtained from other sources, such as a disk.

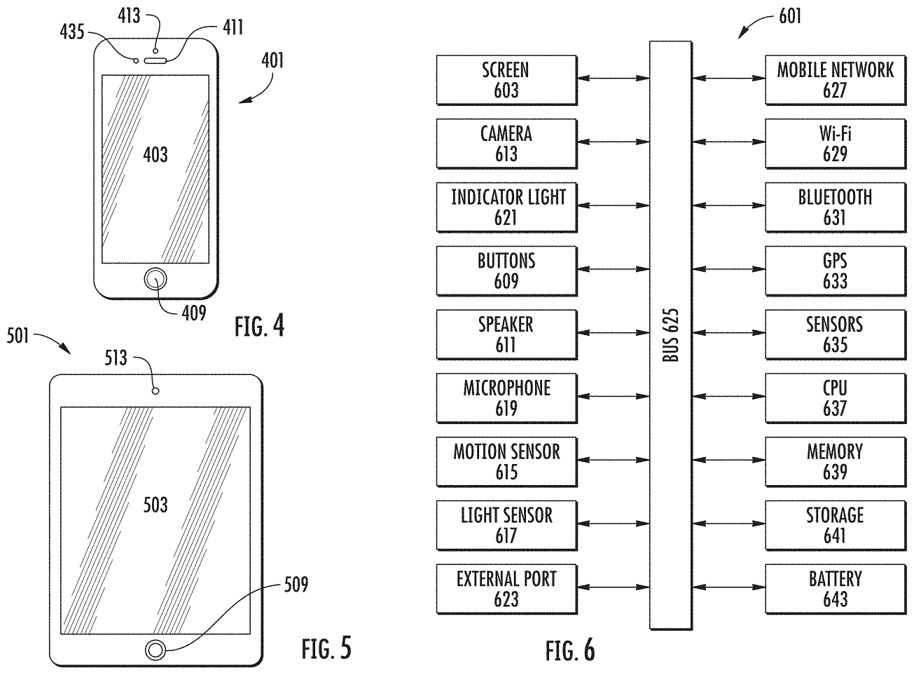

FIGS. 4-5 show examples of mobile devices, which can be mobile clients. Mobile devices are specific implementations of a computer, such as described above. FIG. 4 shows a smartphone device 401, and FIG. 5 shows a tablet device 501. Some examples of smartphones include the Apple iPhone, Samsung Galaxy, and Google Nexus family of devices. Some examples of tablet devices include the Apple iPad, Apple iPad Pro, Samsung Galaxy Tab, and Google Nexus family of devices.

Smartphone 401 has an enclosure that includes a screen 403, button 409, speaker 411, camera 413, and proximity sensor 435. The screen can be a touch screen that detects and accepts input from finger touch or a stylus. The technology of the touch screen can be a resistive, capacitive, infrared grid, optical imaging, or pressure-sensitive, dispersive signal, acoustic pulse recognition, or others. The touch screen is screen and a user input device interface that acts as a mouse and keyboard of a computer.

Button 409 is sometimes referred to as a home button and is used to exit a program and return the user to the home screen. The phone may also include other buttons (not shown) such as volume buttons and on-off button on a side. The proximity detector can detect a user's face is close to the phone, and can disable the phone screen and its touch sensor, so that there may be no false inputs from the user's face being next to screen when talking.

Tablet 501 is similar to a smartphone. Tablet 501 has an enclosure that includes a screen 503, button 509, and camera 513. Typically the screen (e.g., touch screen) of a tablet is larger than a smartphone, usually 7, 8, 9, 1, 3, 4, or more inches (measured diagonally).

FIG. 6 shows a system block diagram of mobile device 601 used to execute the software of the present invention. This block diagram is representative of the components of smartphone or tablet device. The mobile device system includes a screen 603 (e.g., touch screen), buttons 609, speaker 611, camera 613, motion sensor 615, light sensor 617, microphone 619, indicator light 621, and external port 623 (e.g., USB port or Apple Lightning port). These components can communicate with each other via a bus 625.

The system includes wireless components such as a mobile network connection 627 (e.g., mobile telephone or mobile data), Wi-Fi 629, Bluetooth 631, GPS 633 (e.g., detect GPS positioning), other sensors 635 such as a proximity sensor, CPU 637, RAM memory 639, storage 641 (e.g., nonvolatile memory), and battery 643 (lithium ion or lithium polymer cell). The battery supplies power to the electronic components and is rechargeable, which allows the system to be mobile.

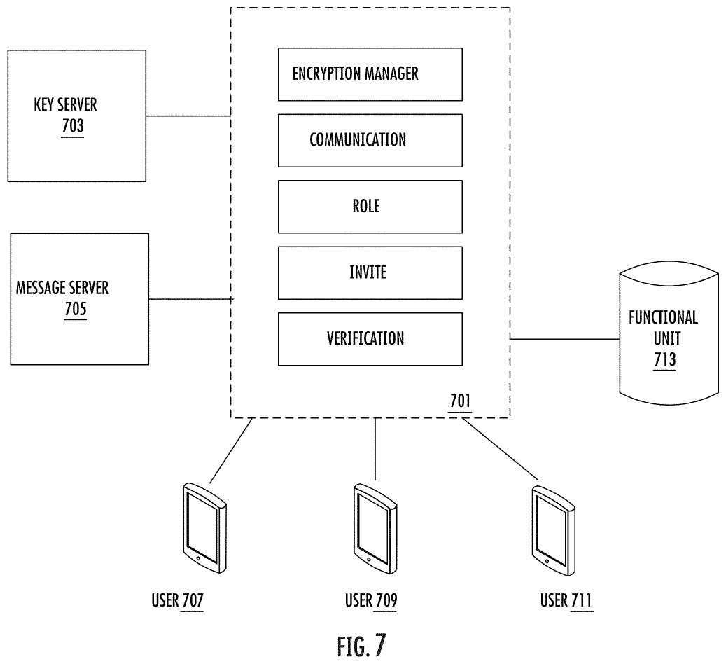

FIG. 7 shows a system diagram of the encrypted messaging system, in an implementation. The encrypted messaging system includes a encrypted messaging system server 701 that is communicatively coupled to a key server 703 and a message server 705. The key server 703 and the message server 705 are provide encryption/decryption keys and messages, respectively to support various features provided by the encrypted messaging system server 701. For example, the encrypted messaging system server may include an encryption manager feature that manages encryption and decryption of messages stored in the message server 705 using keys stored in the key server 703.

A communication feature allows communication between people using the encrypted messaging system. For example, the users may be users 707, 709, or 711. The people do not need to be registered or verified users to use the encrypted messaging system (e.g., newly invited user to join the encrypted messaging system). further, the communication feature may facilitate different types of communication methods in the encrypted messaging system. For example, the encrypted messaging system may provide chat, email, voice-over-internet-protocol, photo, video, or other types of communication methods.

A role feature allows the encrypted messaging system to enforce access control to features of the encrypted messaging system. For example, role may be related to space or functional units defined in a database 713. Certain spaces may have access to certain features that other roles may not. For example, a back office may have access to accounting features but not medical records. As another example, a patient should not have access to information of other users, such as medical information of other patients or messaging features that represent themselves as medical professionals. An invite feature allows existing users of the encrypted messaging system to invite others to join the encrypted messaging system. Invited persons may be associated with any role of the encrypted messaging system, such as staff, doctor, patient, or other roles.

A verification feature assists the encrypted messaging system in determining whether a person is actually the person they allege to be. For example, the encrypted messaging system would verify whether users are actually medical practitioners, so that the encrypted messaging system is not misused by those impersonating medical practitioners for financial gain.

Example Messaging Uses

Some examples of some use cases of the encrypted messaging system include:

Interoffice communication.

Doctors or medical offices often need a method to share information with other doctors or medical offices. For example, interoffice communications include a doctor's office to talk to another doctor's office, such as through staff members of each respective office. With interoffice communication, users communicate with physicians outside of a particular doctor's practice to facilitate referrals, discuss patient issues or just chat with colleagues. Some examples of interoffice communication may include doctor-doctor (from different offices) or staff-staff communications. The encrypted messaging system may refer to any medical business or establishment as a place. For example, a generalist from a first place may be a referrer for a patient to a specialist at a second place. However, since sensitive information may be included in the information doctor's share, the information must be kept confidential. Further, doctors need a way to ensure that contact information used to contact another doctor is valid. For example, contact information may be falsified, incorrect, or outdated. If the contact information is falsified, a malicious party may pose as the referred doctor and obtain sensitive information.

Intraoffice communication.

The encrypted messaging system may include for a medical office different departments with staff that may be responsible for different roles. Examples of intraoffice messaging include communications with one doctor's office, doctors, staff and patient messages and may be associated by history or conversations, and the department. For example, a medical office may include a customer service representative or receptionist, billing specialist, multiple doctors, patients, or other roles. Some examples of intraoffice communication may include doctor-doctor, doctor-staff, or staff-staff communications. Intraoffice communication may also include third-parties a doctor as contracted with to provide service. For example, doctors may choose to outsource a portion of their billing to third-parties. The encrypted messaging system may be configured to allow verified third-parties access to the encrypted messaging system and associate them with applicable spaces (e.g., a third-party billing service may be associated with a back office space). With intraoffice communication, doctor-staff-patient users may communicate with each other. In an implementation, intraoffice communication is separated into different spaces. Like most businesses, a medical office is likely comprised of different departments. To streamline communications within a place, the encrypted messaging system allows users to virtually recreate the structure of their business as different departments or spaces to give users the ability to communicate with the right people every time. For example, a space in the encrypted messaging system refers to a department or subdivision within a particular place. Some examples of spaces in a medical office include “front office,” “back office,” “billing,” or “general.” This also provides a layer of security by ensuring only the people who are meant to view communications are able to.

Spaces may also be used in the encrypted messaging system to define different tasks and who handles the task in a medical office. For example, although a doctor is responsible for the medical care of their patients, they may require the assistance of many others in their office to run an effective medical practice. A doctor may not need to use the encrypted messaging system often, since most tasks in the medical office may be delegated to their respective space (e.g., appointments to the front office, how to pay bills to the back office, and other examples).

There may be different methods to sort through intraoffice messages. For example, messages can be searched by message history or by particular departments at a medical office.

Office to patient communication.

A particular example of intraoffice communication is office to patient communication. The encrypted messaging system provides staff and patients a streamlined avenue of communication—eliminating the need for inefficient phone calls and emails.

When a medical professional logs into the encrypted messaging system, they are asked to create Spaces (or departments) for the business. After staff is added, each staff user can be assigned to one or more departments or spaces they work in normally. This serves several functions:

All communications within each space are secure and cannot be seen by users without access (pursuant to HIPAA);

Users can choose to communicate with an entire department rather than each person individually, saving valuable time;

Patient users can directly communicate with the department/person they need rather than going through a receptionist—allowing medical staff to avoid sorting through emails and time-consuming phone calls; and

Different types of users may be associated with a role, which grants or limits features of the encrypted messaging system, depending on a user's respective role. An example may be limiting users with the billing role from viewing medical test results.

The encrypted messaging system may include chat features. This allows different users of the encrypted messaging system to communicate with as many other users as desired, such as through one-to-one messaging or one-to-many messaging. In an implementation, the chat feature does not include a presending feature. This means that typed text is not transferred from a user's device before the entire message has been drafted. This allows the encrypted messaging system to ensure incomplete messages are properly secured for HIPAA compliance.

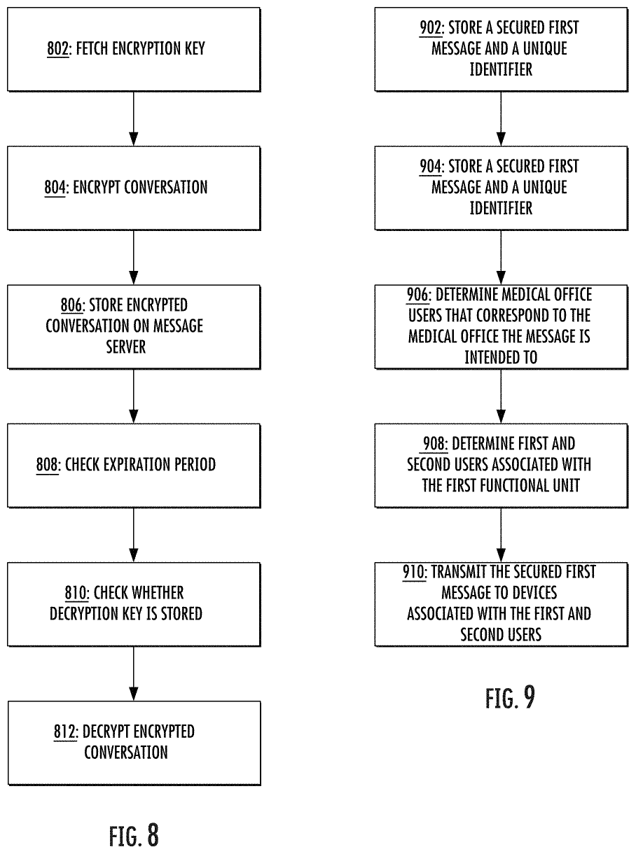

FIG. 8 shows a flow of encrypting messages in the encrypted messaging system, in an implementation. This application includes flows including a series of steps, however these flows are for illustrative purposes only. Various embodiments of the encrypted messaging system may include greater or fewer steps than shown by flows included.

In a step 802, the encrypted messaging system includes fetching an encryption key. The encryption key may be an encryption key that has been used previously in the encrypted messaging system, but uniquely associated with a sender of a conversation that is to be encrypted using the first encryption key. The sender may associate the conversation for a particular functional unit, such as a back office, staff, front office, or other space or functional unit. The particular functional unit corresponds to one or more users of a medical office. Users of the medical office but not associated with the first functional unit not allow access to the conversation.

In a step 804, the encrypted messaging system includes encrypting the conversation using the encryption key. Information associated with the conversation may be stored. For example, the encrypted conversation may be associated with a unique identifier that allows the encrypted messaging system to identify the conversation as a conversation with the sender, even when the conversation is encrypted. A timestamp of the message may also be included.

In a step 806, the encrypted messaging system includes storing the encrypted conversation on a message server. The message server may be separate from a key server storing keys used by the encrypted messaging system.

In a step 808, the encrypted messaging system includes checking an expiration period for the conversation. For example, using the timestamp of the conversation, the encrypted messaging system may determine whether the conversation exists in the encrypted messaging system or has expired. For example, the encrypted messaging system may delete encrypted conversations if a timeout period for the conversation has expired.

In a step 810, the encrypted messaging system includes checking whether a decryption key is stored. For example, if the encrypted conversation is being read on a device with the application decryption key already stored, the encrypted messaging system may not need to transfer the decryption key. Otherwise, the decryption key is transmitted to a device for decryption of the encrypted conversation. In a step 812, the encrypted messaging system includes decrypting an encrypted conversation.

FIG. 9 shows a flow for messaging functional spaces in the encrypted messaging system, in an implementation. In a step 902, the encrypted messaging system includes receiving from a patient user's device a request to secure a message. The message may also specify a functional unit and a medical office the message is intended for.

In a step 904, the encrypted messaging system includes storing a secured first message and a unique identifier. For example, the user's message may be encrypted using a selected encryption key.

In a step 906, the encrypted messaging system includes determining medical office users that correspond to the medical office the message is intended to. For example, the encrypted messaging system may provide services to one or more medical offices. Each medical office needs to maintain confidentiality of their respective information, even if a user is a patient of more than one medical office, unless the information is intended to be shared. Users may be associated with the medical office, such as employees, doctors, staff, contractors or others associated with the medical office.

In a step 908, the encrypted messaging system includes determining first and second users of the secured messaging system associated with the first functional unit. For example, not all users associated with a medical office may be doctors, staff, accounting, or other roles designated in the encrypted messaging system. Each functional unit may only have access to all or limited information in the encrypted messaging system.

In a step 910, the encrypted messaging system includes transmitting the secured first message to devices associated with the first and second users.

Encryption

In an implementation, the encrypted messaging system includes various methods to access communications. The encrypted messaging system may allow users to access the encrypted messaging system using their mobile devices or browsers. This provides users a device agnostic-capability to access the encrypted messaging system, while providing access to various features of the encrypted messaging system, such as interoffice and intraoffice messaging.

In an implementation, the encrypted messaging system stores a copy of keys used to encrypt and decrypt communications in the encrypted messaging system on a key server. This may allow the encrypted messaging system to access communications in emergency situations, such as when there is a medical emergency and communications made in the encrypted messaging system may assist in discovering what happened. Alternate implementations of the encrypted messaging system may also include end-to-end encryption for communications in the encrypted messaging system. For example, the encrypted messaging system may be adapted for use in industries other than the medical industry, such as finance. Depending on requirements of each industry, the encrypted messaging system may use end-to-end encryption to allow only users that send a message and users intended to receive the message to open and decrypt the message.

In an implementation, messages sent in the encrypted messaging system include an expiration period. For example, non-encrypted or encrypted messages may be associated with a time they are sent. The expiration period may be any length of time (e.g., 24-hours, 48-hours, 3 days, or any other time period). When the expiration period calculated using the time sent has been reached, the encrypted messaging system may delete either copies of the message from a message server or client device, or a key used to decrypt the message stored on the encrypted messaging system's server. Optionally, copies of the key or message may also be deleted when the expiration period has been reached.

The encrypted messaging system may include automatic logout features. For example, a user logged into a device may be logged out automatically after a logout condition has occurred. Some examples of log out conditions include a predetermined amount of time logged on, a predetermined amount of idle time while connected to the encrypted messaging system, when a device has been shut off or enters sleep mode, or a combination of any of these.

The encrypted messaging system incorporating HIPAA compliant communications can include one or more computers to control or access information from. FIG. 1 shows an example of a computer that is component of an encrypted messaging system. The computer may be a separate unit that is connected to a system, or may be embedded in electronics of the system. In an embodiment, the invention includes software that executes on a computer workstation system or server, such as shown in FIG. 1.

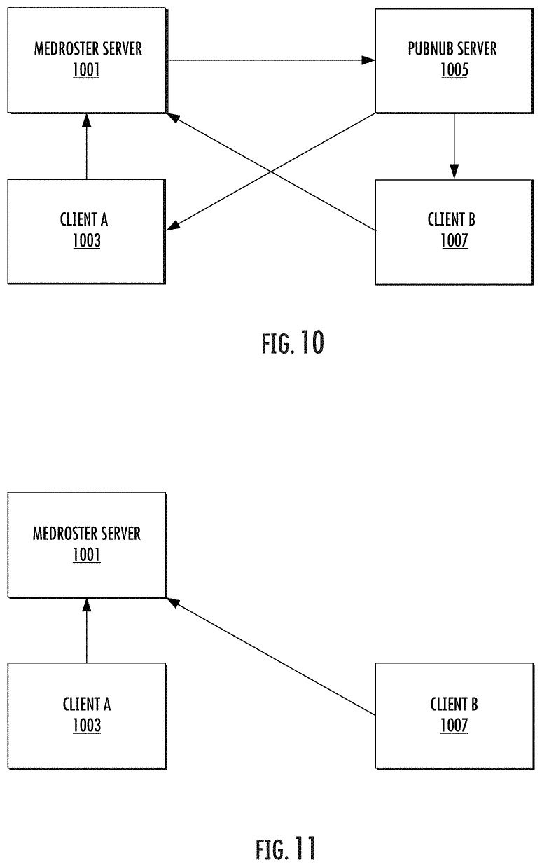

FIG. 10 includes information on an encryption feature for an encrypted messaging system. The encryption feature may be implemented for various pieces of information stored in the encrypted messaging system, such as messages, chats, patient information, contact information, or any other piece of information in the encrypted messaging system. The encryption features may provide encryption for data stored in the encrypted messaging system while the data is at rest and when the data is in motion.

Implementations of the encrypted messaging system may use different types of encryption. Some examples of encryption that may be used by the encrypted messaging system include (1) key reservation encryption, (2) end-to-end encryption, or (3) hybrid encryption.

In an implementation where the encrypted messaging system uses end-to-end encryption, this means that only authenticated users included as a recipient or sender of an encrypted message may decode the encrypted message. For example, a user's device may store keys used by the user. The Medroster server does not retain a copy of a key when end-to-end encrypted messaging is used.

In an implementation where the encrypted messaging system uses hybrid encryption, end-to-end encryption or key reservation encryption may be used for all or a subset of messages of the encrypted messaging system. For example, encrypted messages for a chat group within a medical office may not require end-to-end encryption or key reservation encryption since it is likely to be focused on more mundane matters within the medical office, such as lunch plans or coordinating events within the office.

In an implementation where the encrypted messaging system uses key reservation encryption, FIG. 10 shows a chat encryption overview for the encrypted messaging system. The overview includes a server 1001 (e.g., a server running software provided by Medroster.com Corporation), a PubNub Server 1005, and client devices A 1003 and B 1007. The PubNub Server 1005 may be any server capable of supporting real-time publish/subscribe messaging. The messaging features includes an application programming interface that may be called by the encrypted messaging system, to facilitate messaging in the encrypted messaging system. For example, the overview shows how a user at client A 1003 may transmit a message using the encrypted messaging system to another user at client B 1007. The overview includes the following steps:

Step 1: client A 1003 sends plaintext message over encrypted connection (https) to Medroster server 1001. The message is intended for a user at client B 1007.

Step 2: Medroster server 1001 fetches key for conversation A<->B from database or creates it if missing and stores it.

Step 3: Medroster server 1001 encrypts the client message using the conversation key and sends the encrypted message to PubNub Server 1005—including a secure random conversation identifier. In an implementation, the secure random conversation identifier is generated by the Medroster server 1001. The Medroster server 1001 does not keep a copy of the message, including the encrypted and the unencrypted version. However, the secure random conversation identifier uniquely identifies the message in encrypted form.

Step 4: PubNub Server 1005 receives the encrypted message and pushes it to Client B 1007. At the same time, pushes acknowledgment of delivery to client A 1003.

Step 5: client B 1007 receive the encrypted message. If already cached locally, client B 1007 uses the key corresponding to the conversation identifier ID to decrypt the message.

Step 6: If the key for the conversation identifier is missing locally, client B 1007 requests the conversation key from the Medroster server 1001 over encrypted connection (https) and decrypts the message.

FIG. 11 shows characteristics of the encrypted messaging system when sending an encrypted message in the encrypted messaging system with a client A 1003 to Medroster server 1001 to client B 1007. Some characteristics include:
Encryption is not end-to-end. The Medroster server 1001 manages and stores the keys
Message sending is protected by the underlying Transport Layer Security (TLS)-encrypted connection
Keys sent by the server to clients are protected by the underlying TLS connection, too
Decryption of messages happens on the client only
Each key is associated with a conversation that can be one-to-one or a group chat
Access to keys is restricted via access permissions to conversations
The keys are stored in encrypted form (as compared to Data-At-Rest Encryption)

In an implementation, messages sent in the encrypted messaging system include an expiration period. For example, non-encrypted or encrypted messages may be associated with a time they are sent. The expiration period may be any length of time (e.g., 24-hours, 48-hours, 3 days, or any other time period). When the expiration period calculated using the time sent has been reached, the encrypted messaging system may delete either copies of the message or a key used to decrypt the message stored on Medroster server 1001. Optionally, copies of the key or message may also be deleted when the expiration period has been reached.

FIG. 12 shows characteristics of the encrypted messaging system when sending an encrypted message in the encrypted messaging system with Medroster server 1001 to PubNub Server 1005. Some characteristics include:
PubNub Server 1005 is limited to encrypted content
The plaintext information they see is the conversation identifier
The conversation identifier is generated as a secure random number converted to Base64.
Keys are created as secure random bytes, non-guessable
This leaves no chance of guessing the participants of a conversation or the key
Due to the properties of the Authenticated Encryption scheme used for message encryption, PubNub may not be able to modify encrypted content without clients detecting (and rejecting) these changes
While the Medroster server 1001 has access to plaintext messages, they are not persistent. For example, plaintext messages in Medroster server 1001 may be subject to one or more safeguards, such as automated deletion, deletion when a time duration has passed, or other methods.
PubNub persists messages, but in encrypted form

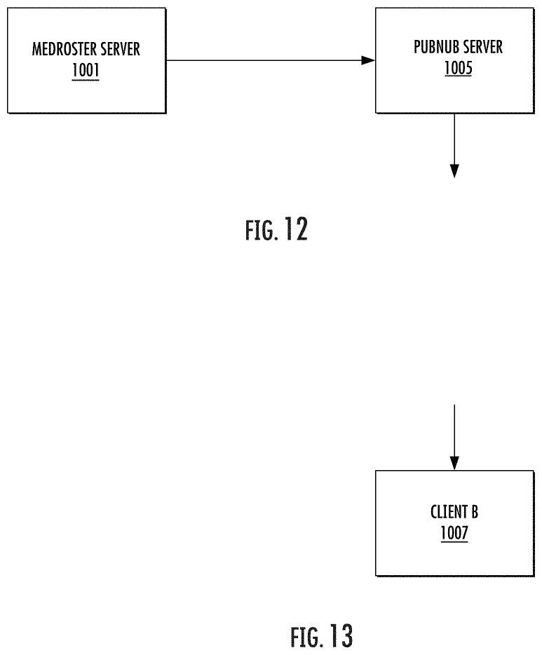

FIG. 13 shows characteristics of the encrypted messaging system when sending an encrypted message in the encrypted messaging system with client B 1007. Some characteristics include:
Client caches the encryption keys locally, but only in memory
No key is ever persisted in any form on the client
No message is ever persisted in any form on the client
Authenticated Encryption: AES-256-ENC with a secure random initialization vector (IV) & HMAC-SHA256
Each conversation key actually consists of two separate keys or tags: One for encryption and one for the message authentication code (MAC)
The IV and the MAC tags are included in the encrypted messages

Table 1 below shows how data is handled in the encrypted messaging system when the data is at rest. For example, a table is shown in the figure storing data of nine users. For each user, a user identifier, name, phone, email, and type is stored. The encrypted messaging system stores sensitive information encrypted on the application-level on a per-column basis. Authenticated Encryption is used (AES-256-ENC with HMAC-SHA256) to store the information, with a secure random IV. A separate encryption and authentication key are derived from the Rails application secret.

TABLE 1

| Identifier | Name | Phone | Email | Type |
|---|---|---|---|---|
| 1 | Yd!w7/i. . . | 23BDSfa . . . | AGf3s2a . . . | DOCTOR |
| 2 | . . . | . . . | . . . | PATIENT |
| 3 | . . . | . . . | . . . | STAFF |
| 4 | . . . | . . . | . . . | DOCTOR |
| 5 | . . . | . . . | . . . | STAFF |
| 6 | . . . | . . . | . . . | STAFF |
| 7 | . . . | . . . | . . . | PATIENT |
| 8 | . . . | . . . | . . . | PATIENT |
| 9 | . . . | . . . | . . . | PATIENT |

EMAIL is typically a field that is used to look up records, such as the table 1 shown above. For example, when a search for user information is made, the email of the user is usually used to identify the user in the encrypted messaging system. Authenticated Encryption may not work here as it is non-deterministic, which causes lookup failure. The encrypted messaging system may use AES in synthetic initialization vector (SIV) mode to guarantee the best tradeoff between security and determinism. With AES-SIV exact-match search is possible, range, order, etc. queries may not be possible.

Table 1 shows how name data is handled in the encrypted messaging system when the data is at rest. Columns not used in queries may be encrypted using AES in ENC mode using a secure random IV for each entry to guarantee the best possible security. For example, name data may be encrypted using AES. Both searchable and non-searchable columns are authenticated with an HMACSHA256 tag. The IV and the MAC tag are stored along with the encrypted value.

In an implementation, chatkey, invitation, and user data is handled in the encrypted messaging system when the data is at rest. For chatkey data, authentication may be non-searchable (authentication key of a single conversation) and encryption—non-searchable (encryption key of a single conversation). For invitation data, email—searchable, mobile_phone data—searchable, invitee_type—searchable (e.g., Doctor, Patient, Staff), first_name—non-searchable, and last_name—non-searchable. For user data, email—searchable, phone_number—searchable, unconfirmed_email—searchable, first_name—non-searchable, last_name—non-searchable, title—non-searchable, and specialty—non-searchable.

Delegated Use

In an implementation, although the encrypted messaging system is designed for use in a medical office or place, doctors may be infrequent users of the encrypted messaging system. For example, staff associated with a doctor may use the encrypted messaging system a majority of the time. Patients and doctors may access the encrypted messaging system also, but spend less time using the encrypted messaging system than staff. For example, a single doctor may have thousands of patients depending on their practice. Not all of these patients may require medical care at any given point in time, so that the bulk of the doctor's patients may not need to use the encrypted messaging system.

Further, not every message from or to a patient may require a doctor's direct involvement. As an example, doctors may need to coordinate with patients to schedule appointments. However, a doctor rarely would access the encrypted messaging system themselves to schedule an appointment. Staff may transmit, on behalf of the doctor, messages using the encrypted messaging system to coordinate with patients. This reduces the workload on the doctor, so that they can focus on providing medical care to their patients.

The encrypted messaging system allows staff to act on behalf of the doctor, while maintaining accountability for each particular user. For example, the messages may indicate that the medical office, medical space, particular staff member, or any combination of these as the source of the message, even when the message is sent on behalf of the doctor. In an implementation, all users of the encrypted messaging system have an individual and verified account with the encrypted messaging system. This means that, although a user may be sending messages on behalf of a doctor user, a medical place, a medical space, or other grouping of users, the identity of the actual user is included with messages. For example, a message from a staff user A on behalf of a front office to a patient user may indicate both that user A and the front office space are responsible for the message. When the patient user views the message, the encrypted messaging system indicates that the message is sent from the user A on behalf of the doctor user from the front office space. If, for example, user A is on vacation or otherwise unavailable to respond, a response from the patient user may be transmitted to user A and other users of the front office space. A user B of the front office space may then continue handling the conversation, even though they were not the initial user A whom sent the first message. The user B's response may then indicate that they are the sender of the message on behalf of the front office. The user A may also receive indication that user B has responded to the patient user's message. In this situation, users A and B are acting as proxies to the doctor user; the doctor user may not need to be notified of these messages, even when other users are acting of their behalf.

In an implementation, the encrypted messaging system may include badges to indicate various pieces of important information. For example, a badge may be used to identify a particular doctor a user is corresponding on behalf of. For example, a shared medical office may include two or more doctors. The shared medical office may share one or more staff users between the two or more doctors. When a staff user is included with messages in the encrypted messaging system, the message may include a badge for the staff user, indicating which doctor of the shared medical office they are corresponding on behalf of

Screens

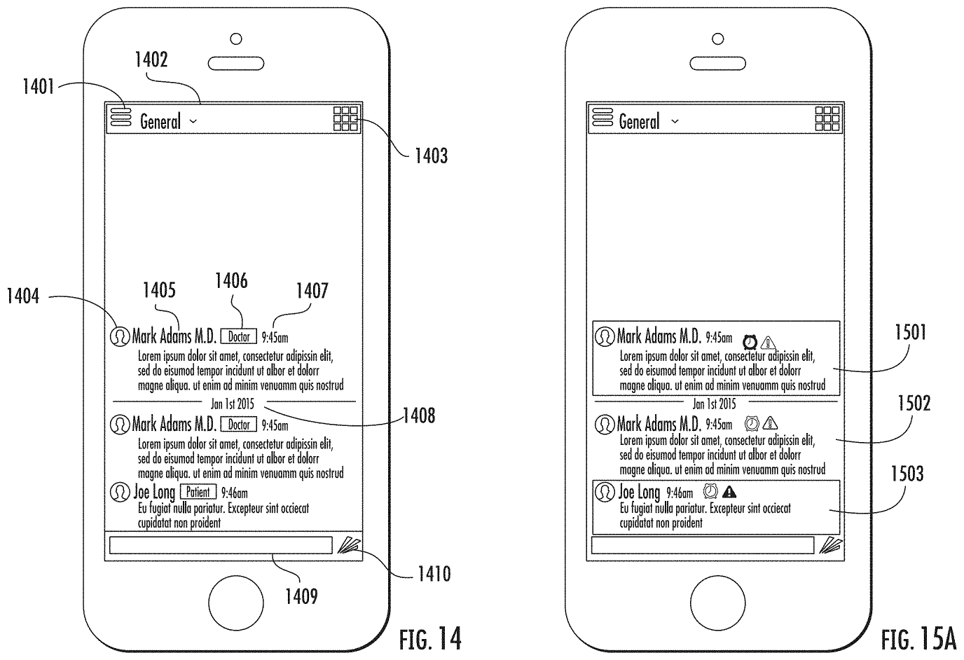

FIGS. 14-39 show screens of the encrypted messaging system when accessed on an application executing on a mobile device. The screens are of a mobile device, such as Apple Inc.'s iPhone®, but other mobile devices or computing devices may be used by a user to access information of the encrypted messaging system. Different types of users may access the encrypted messaging system. The screens may be from an application designed to be a sleek and efficient messaging platform that mirrors a related web application.

In an implementation, the encrypted messaging system (1) allows HIPAA-compliant one-to-one chat between a person or office (2) facilitates intraoffice communications such as allowing staff to act on behalf of a doctor, without the doctor's express supervision (e.g., the encrypted messaging system may be used mostly by staff and patients). The encrypted messaging system may be divided into different parts. A medical place in the encrypted messaging system may be a medical office, business entity, a HIPAA compliant unit, or, in larger medical establishments, a department or any division used by the medical industry.

The medical office may include multiple medical spaces, such as sections or departments within a place or a doctor's office. Some examples of medical spaces include front office, back office, reception, billing, doctor, other staff, or any other space in a medical office. In an implementation, for larger institutions such as a hospital, the medical place may be defined as a department in a hospital, for manageability and ease of use of the encrypted messaging system.

For example, the encrypted messaging system may be accessed by a doctor 5% staff 85% patient 10% of the time. FIG. 14 shows a screen in a places chat. The “Place Chat” interface (or the “General” Space Chat) is the first screen a user may see when they login to the encrypted messaging system. “Place Chat” is open to all users belonging to that particular place and cannot be deleted. “Place Chat” is a messaging interface for all staff non-patient users in a place to communicate with each other without patients being able to see. No patients can access “Place Chat.” FIG. 14 includes:

1401. “Left Slide Out”: This menu button when pressed may function by sliding the screen to the right, exposing the left-docked menu. Pressing this may take the user to the “Left Menu Spec”

1402. “Convo Title Button”: This feature may display the conversation title on each chat screen interface. Depending on which conversation style the user has selected, it may change. When speaking to a user one-on-one, the conversation title should display that user's name. When inside a place/space, the title of that place/space should appear. When pressed, this button may display the users taking part in this place/space/group message

1403. “Menu Slide out”: When pressed, this “contact” button may function by sliding the screen to the left, exposing the left-docked contact list

1404. User Image

1405. User Name

1406. “Badge” (user type Doctor/Staff/Patient)

1407. Timestamp

1408. Datestamp

1409. Text Input Area: Tapping text input area to type causes keyboard to pop up

1410. Send Button: Tapping send button causes message to display for users on the chat interface and hides the keyboard.

A “Left Menu” is accessible from the “Place Chat” interface, as well as any other chat interface. The screen slides right and the menu is viewable. This function is to provide a navigation area for Places, Spaces, and Direct Messages, as well as any other menu items (e.g., referrals, reminders, invites, logout, edit places). A spaces (buttons): direct to the space chat interface (open group chat, but only to users with access to the particular space.)

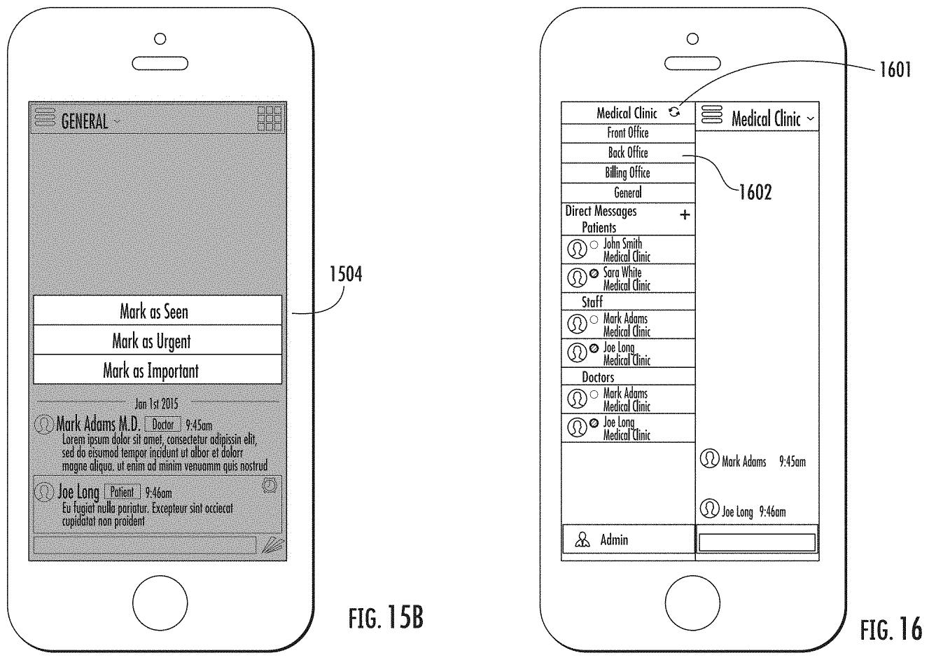

FIGS. 15A-15B show two screens for an urgent/important message. For example, this includes a Direct Messages feature: when a user contacts another user by selecting that user from their contact list, the chat history between those two users may appear. If the menu visible, that users name may appear under direct messages. The functions of this menu are to keep all of the user's conversations efficiently organized. Selecting a users name from direct messages may cause the users shared chat history to appear, the same as contact list. When it is different is the ability to delete the message history direct messages. If the user is a patient, they may be contained under the Patient label and visa versa with colleagues. FIG. 15 includes:

1501. Shows a message marked as urgent

1502. Shows a message marked as urgent

1503. Online/Offline indicator. Displays green when user is online/grey when offline

1504. Shows a menu allowing a message sender to designate a message as seen, urgent, or important.

FIG. 16 shows a screen for changing places. For example, a user may belong to more than one space or place. FIG. 16 includes:

1601. Switch Button

1602. Place Panel: Pressing a different place may open that place up for the user to communicate in. May change all conversation/contacts/settings relevant to that specific place.

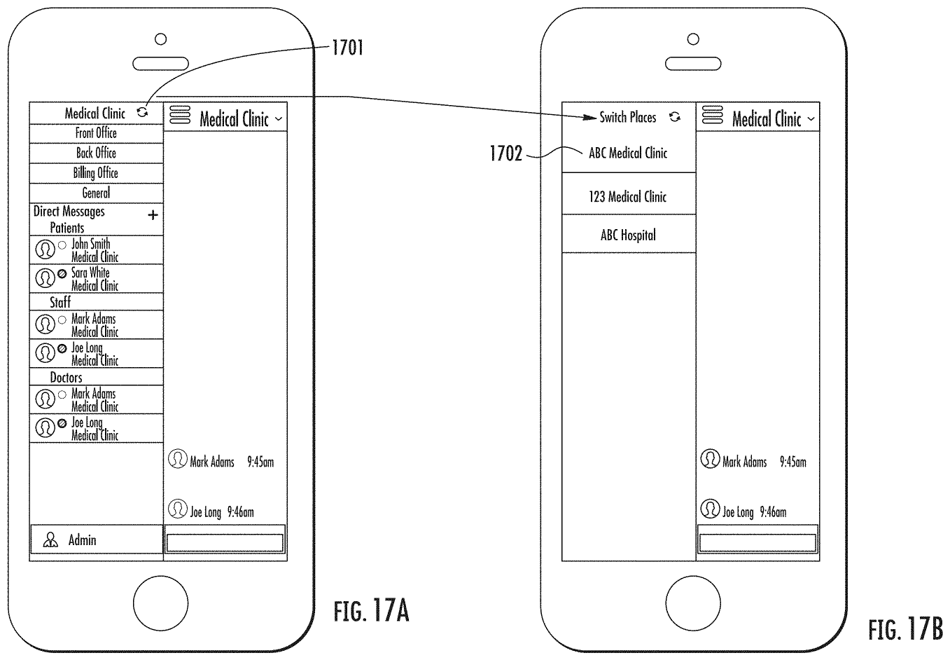

FIGS. 17A-17B show two screens for switching places. The figures include:

1701. Selecting the changing places feature.

1702. Shows places that the user may switch to. These are places that the user belongs to in the encrypted messaging system.

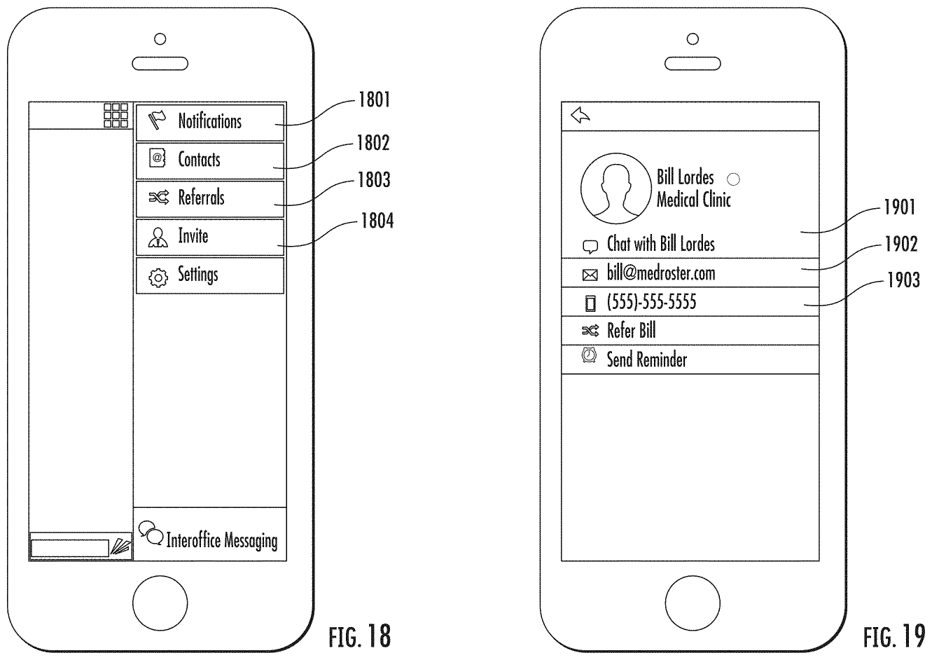

FIG. 18 shows a “Right Menu” function. The “Right Menu” function may be a catch-all for any miscellaneous navigation of the encrypted messaging system, like contacts, settings and referrals etc. Because there different functionalities pertaining to Intraoffice as opposed to interoffice messaging (internal/external), the menu options are different for each. Pressing any button may take the user to the respective screen.

FIG. 18 shows a screen for right menu (e.g., Interoffice). FIG. 18 includes:

1801. Shows notifications

1802. Shows contacts for the user

1803. Shows referrals feature

1804. Shows invite feature.

This may further includes a Contact button filters users; Search may filter users (reference search spec); Online/offline indicator; Pressing user may slide over to show profile view with more detailed contact information (user profile spec).

FIG. 19 shows a screen for a user profile (patient). The user profile may show relevant information pertaining to communicating with a particular patient type user. The patient and doctor/staff profile are similar, but the patient has more options. FIG. 19 includes:

1901. Pressing the “Chat with . . . ” panel may open up a direct message between user A and “Bill Lordes”

1902. May connect to external email application in phone

1903. May open up phone dialer interface in user's phone

The user profile also includes:

Referral—may open up auto filled referral form containing relevant user info (see referral spec)

Reminder—may open up auto filled reminder

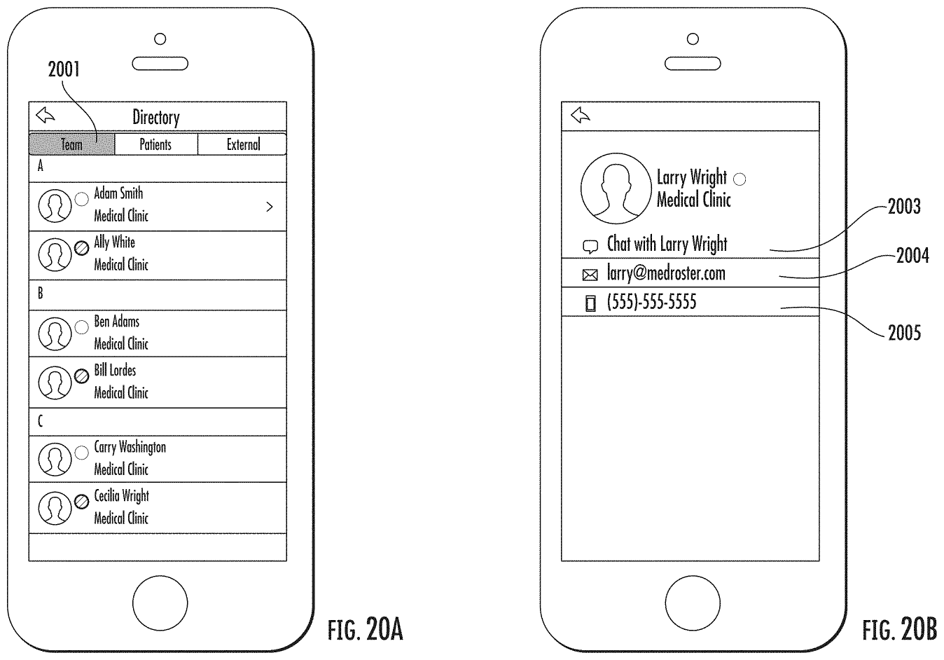

FIGS. 20A-20B show a screen for a user profile (doctor) and a doctor directory screen. For example, the contact list may be separated automatically between your team (doctors and staff that are part of the same place), Patients (patient users that belong to the user's place) and external users (non-patient users who belong to a different place.) All lists may look/function identically. Pressing a user may switch to a user's profile screen. From there, they can choose to contact the user in different ways. For doctor type users, the user may have more options for, including referring the patient to another user and creating reminders for the user. The “refer” button may open up the “create new referrals” screen with data filled in. FIG. 20 includes:

2001. Selecting a team

2002. Show users of the team. A status indicator shows whether the particular user is online

2003. Pressing the “Chat with . . . ” panel may open up a direct message between user A and “Larry Wright”

2004. May connect to external email application in phone

2005. May open up phone dialer interface in user's phone.

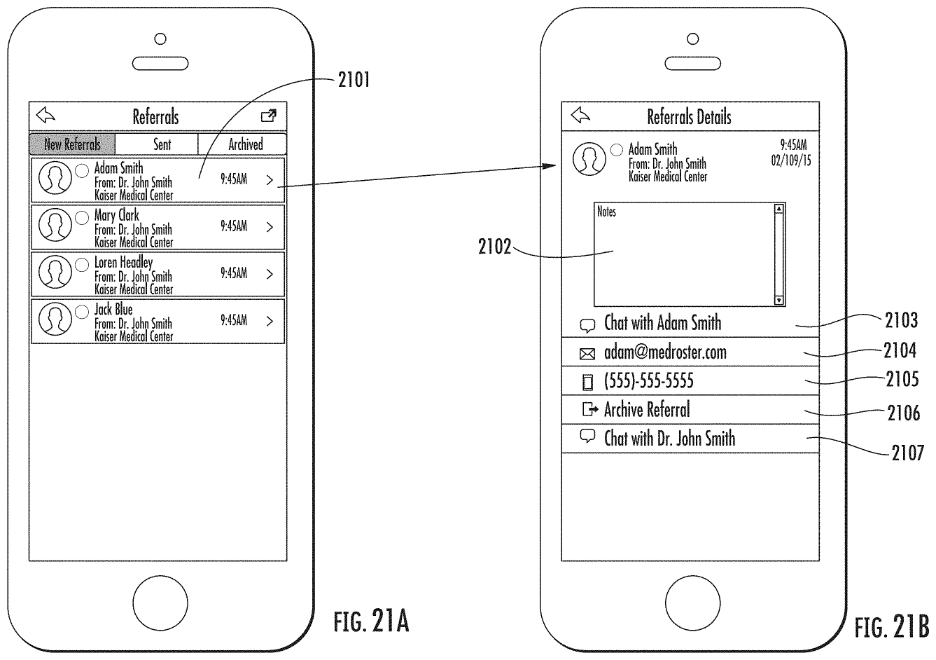

FIGS. 21-21B show screens for received referrals. The referral process entails the act of User A (doctor) sending User B's (patient) information/contact info and details regarding said referral to User C (receiving doctor). When User A sends User C said “referral”, User B is now part of User B's contact list, and vice versa. They now have the ability to communicate further in the same manner that and Doctor user would have to communicate with any other patient user that was part of the Doctor user's place. User C now belongs to both User A and User B's Place. The doctor/staff type users may see a list view of all new/unopened referrals that may work similar to the directory list. The “archived” referral button may switch to archives referrals, which looks exactly the same and new referrals. FIGS. 21-21B includes:

2101. Received referral information panel includes: Patient name and online/offline status, doctor name/location that sent the referral, and a timestamp. Pressing the panel may open up referral details

2102. Any notes from said referral go here

2103. The referral document may automatically generate and display the Patient Users (referred user) contact information. Clicking the “Chat with (User)” link may automatically open a conversation with that user. The chat interface may be displayed

2104. This may open up the user's email system that they user in their phone

2105. This may open using the user's phone dialing system interface

2106. “Archive Referral”: This may remove this specific referral from the “new” tab and move it to the “Archived” tab. It may view and function the same

2107. Each referral received may give an option to communicate with the sending Doctor user for accessibility purposes. The referral form may also display a link that when selected, the receiving user can open a chat with the sending user

2108. Button for creating a new referral.

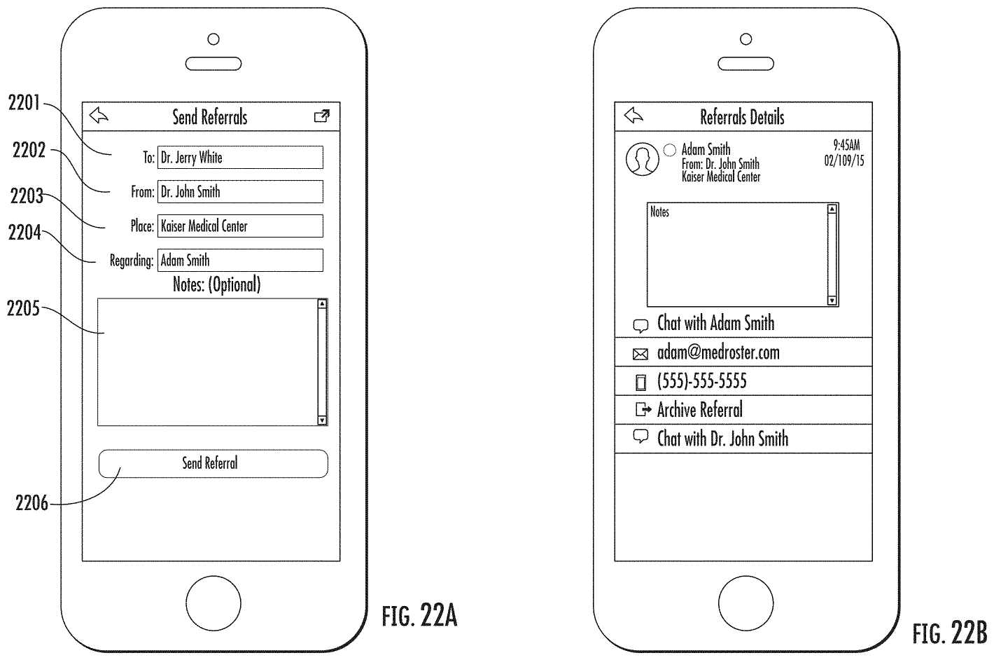

FIGS. 22A-22B show two screens for creating referrals. The encrypted messaging system allows sending referrals. Since this may be used by doctors frequently, it needs to be simple, quick and seamless. FIGS. 22-22B includes:

2201. (Contact List) When selected, choose from Doctor contacts

2202. (Autofill) Sending user info added automatically

2203. (Autofill) Sending users place

2204. (Contact List) Choose from patient contacts

2205. Notes

2206. Sends to user in “TO” form

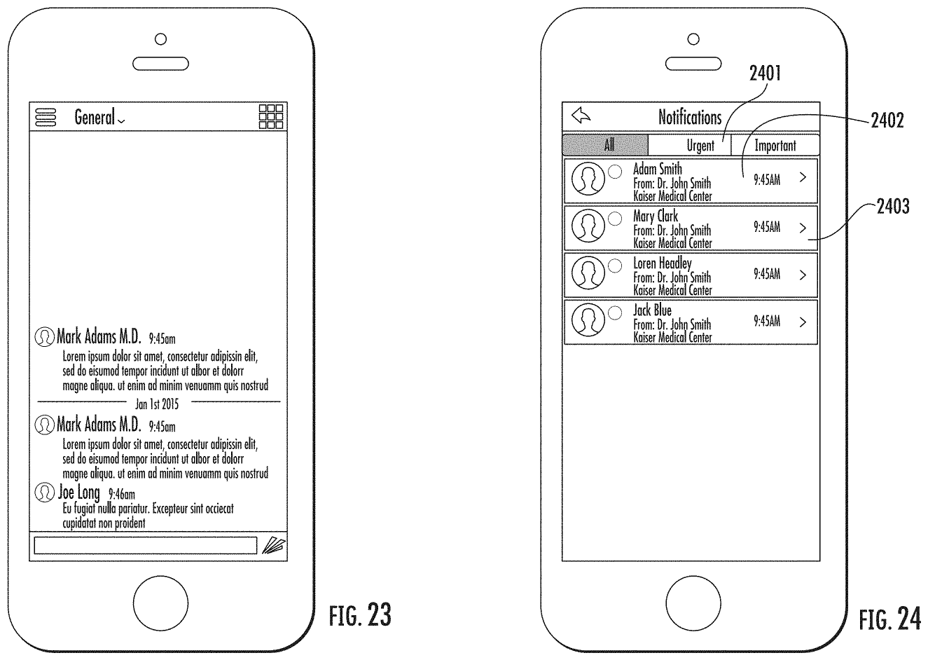

FIGS. 23A-24 show screens for notifications. The notifications may be visible when a user has messages they have yet to view. When messages have not been read, a user may view a green bubble with the number of unread messages on the left menu button. When the button is clicked, the notifications go away. When a user has unread referrals, they may view a red bubble with the number of unread messages on the right contact list menu icon. When it is opened, the notification goes away. FIG. 24 includes:

2401. Switching between “All, Urgent, and Important” may show those messages with that tag in that screen

2402. These are New, Unread messages

2403. Pressing the message summary may open up the message in the chat.

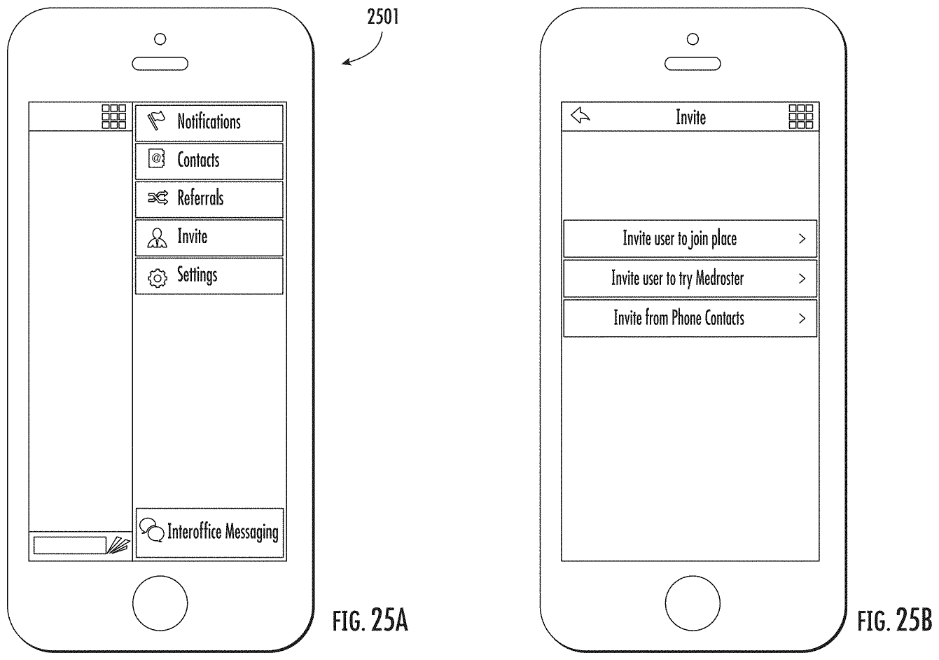

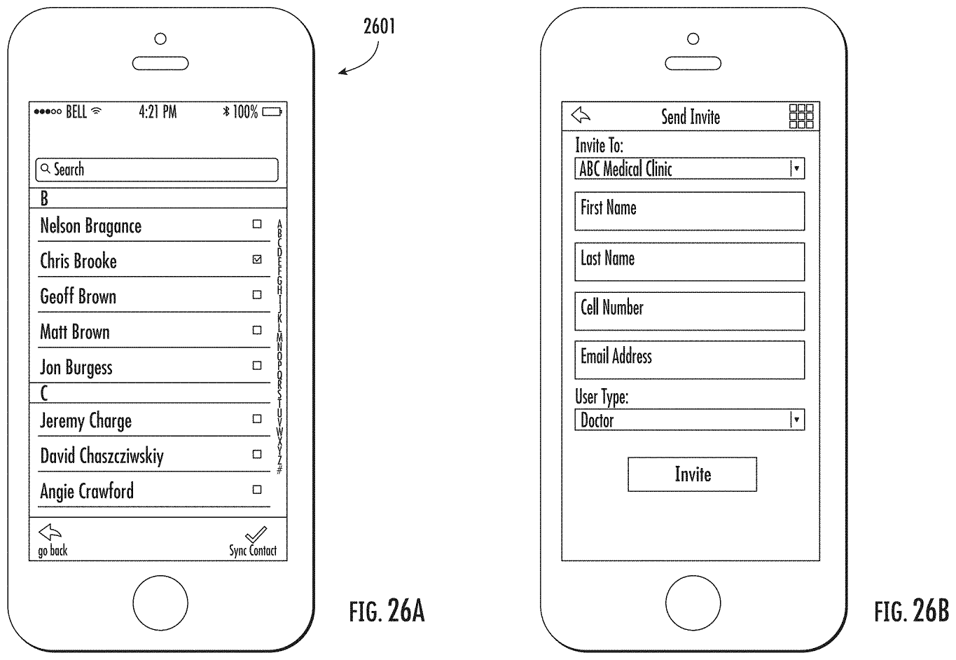

FIGS. 25A-26B shows screens for invitations. An inviting user may choose how to invite new to the encrypted messaging system or existing users to join places, as shown in 2501. As shown in 2601, the inviting user may access a sync contact feature that allows the encrypted messaging system to view contacts in the inviting user's device to invite. The inviting user's invitation may specific where to invite the user (e.g., a medical space) and a user type (e.g., a functional unit).

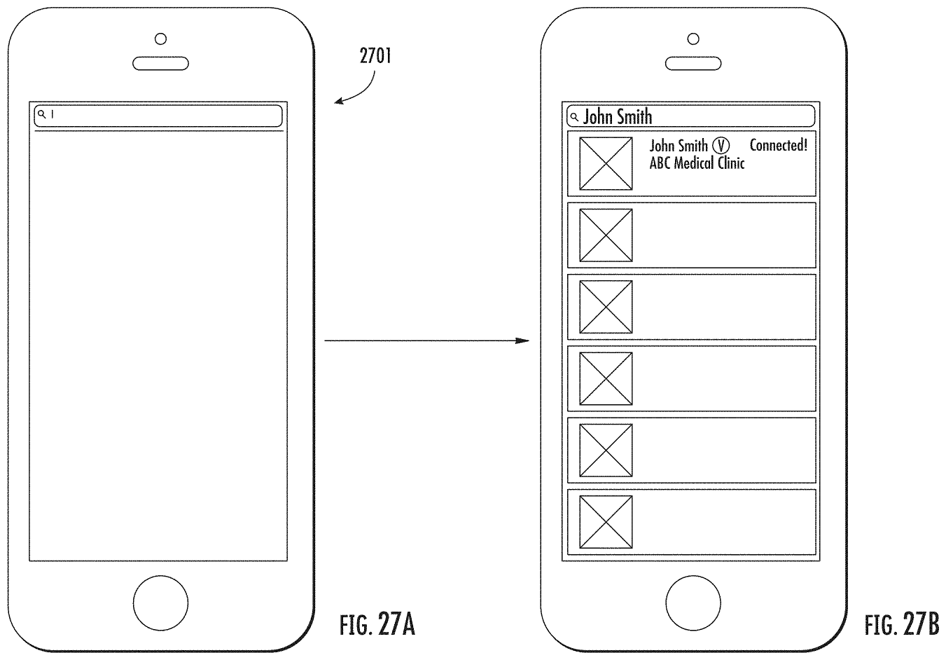

FIGS. 27A-27B show screens for searching for users. When a user is already connected with the current user in the encrypted messaging system, the encrypted messaging system provides a notification that the user is connected, as shown in 2701.

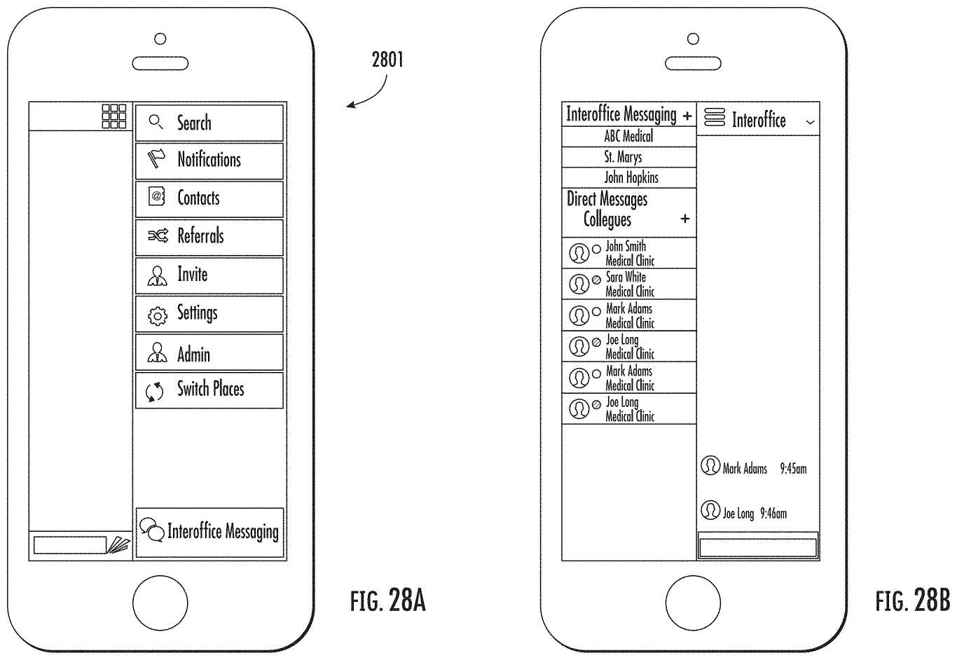

FIGS. 28A-28B shows screens for interoffice messaging, as shown in 2801.

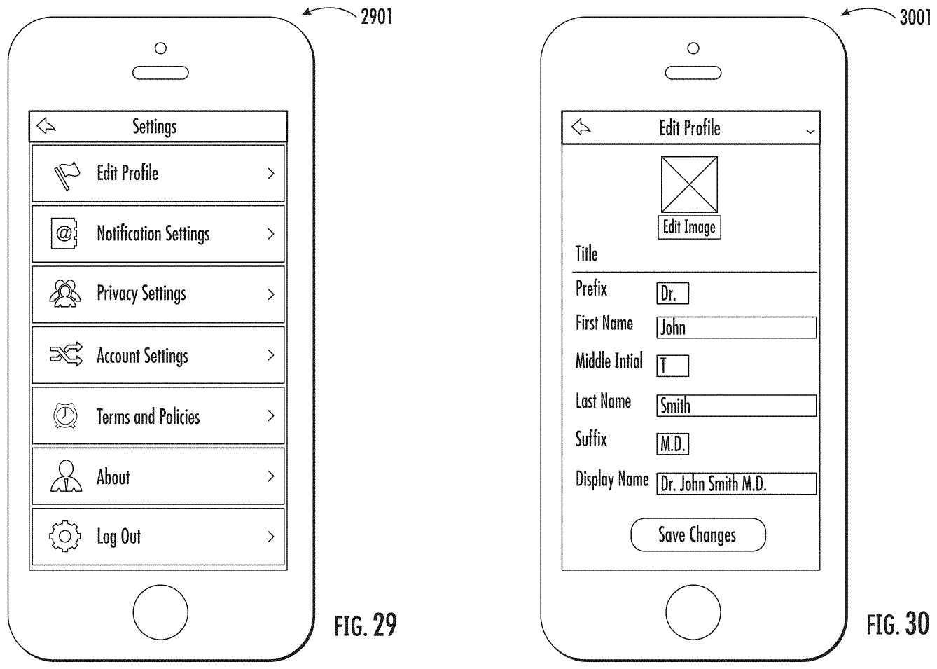

FIG. 29 shows a screen for setting for the encrypted messaging system, as shown in 2901.

FIG. 30 shows a screen for editing a profile for the encrypted messaging system, as shown in 3001.

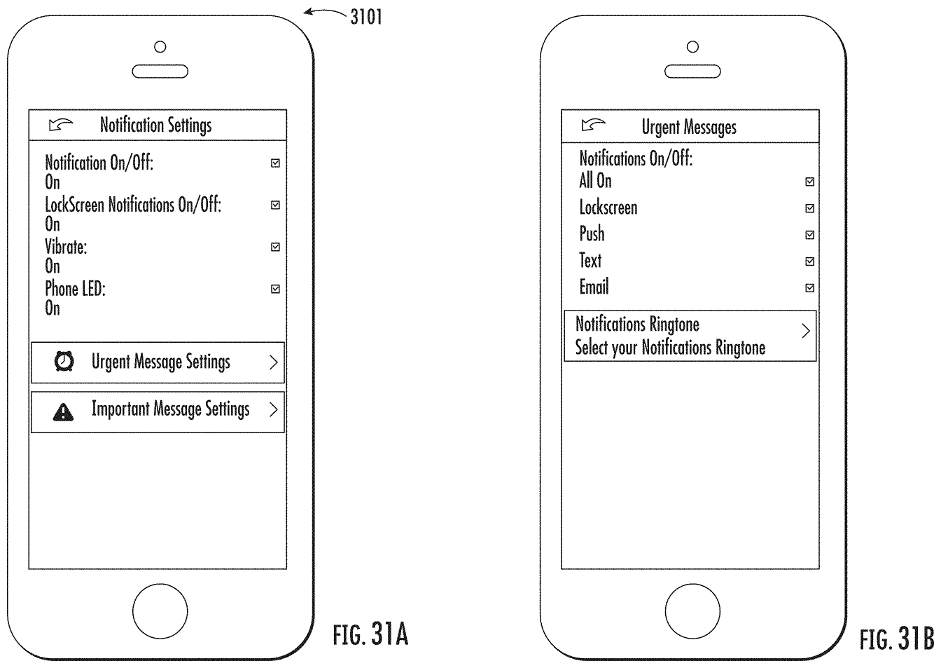

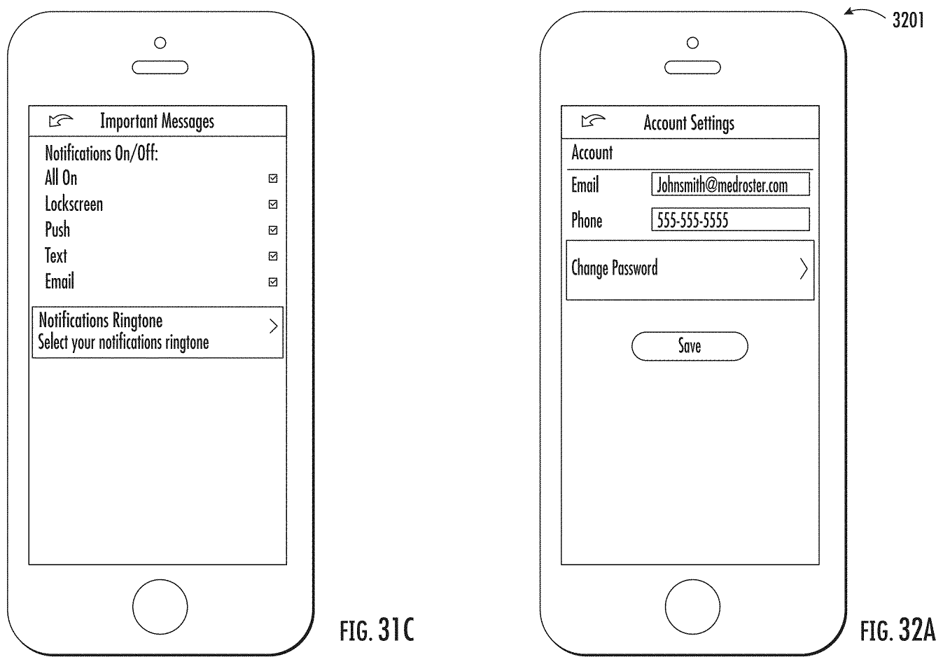

FIGS. 31A-31C show screens for setting notifications in the encrypted messaging system, as shown in 3101.

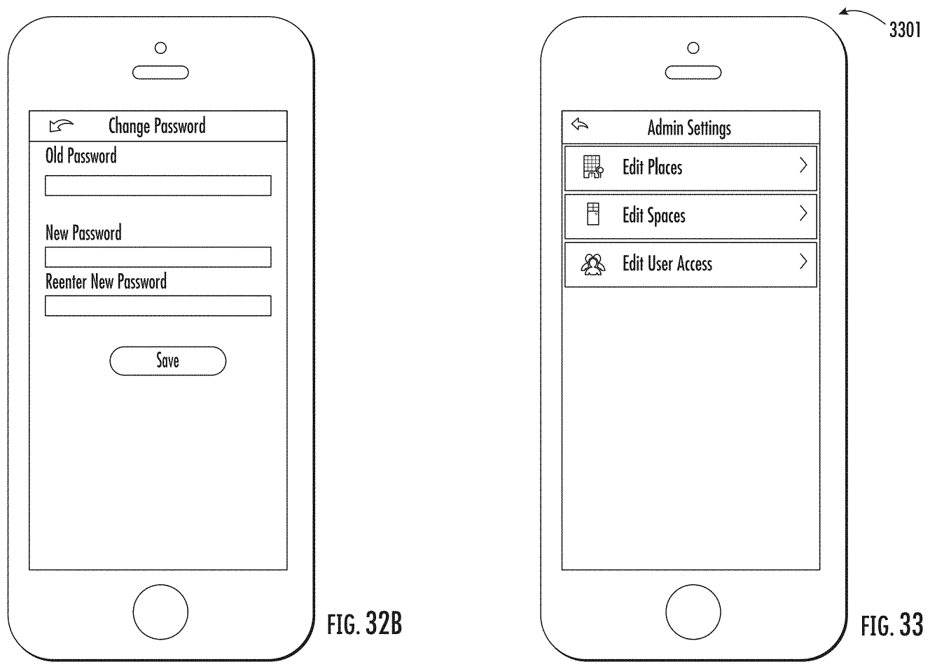

FIGS. 32A-32B show screens for account settings in the encrypted messaging system, as shown in 3201.

FIG. 33 shows a screen for an administrator in the encrypted messaging system, as shown in 3301. The administrator for a place may be a doctor type user or a staff type user.

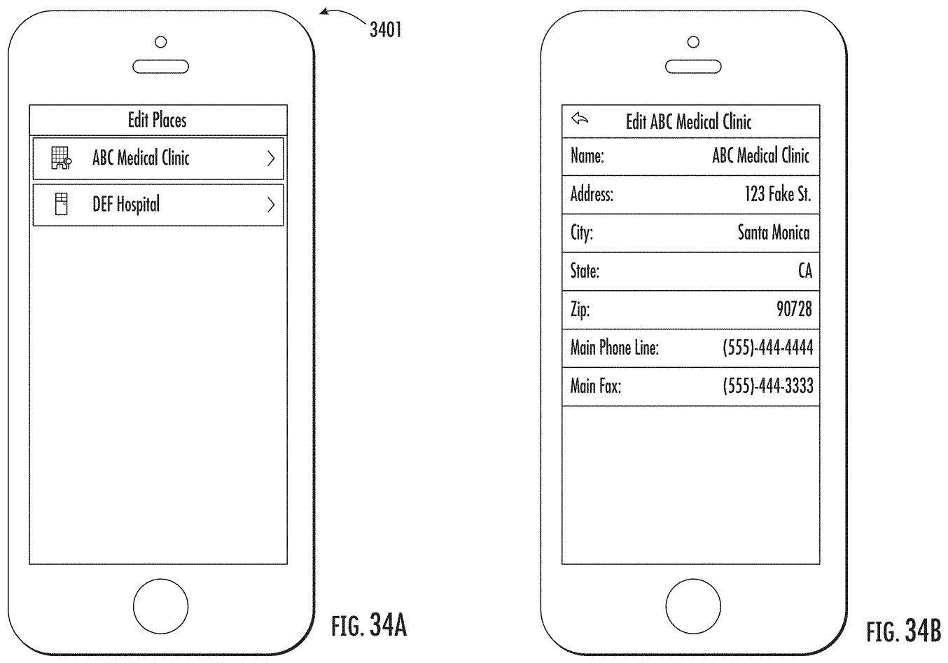

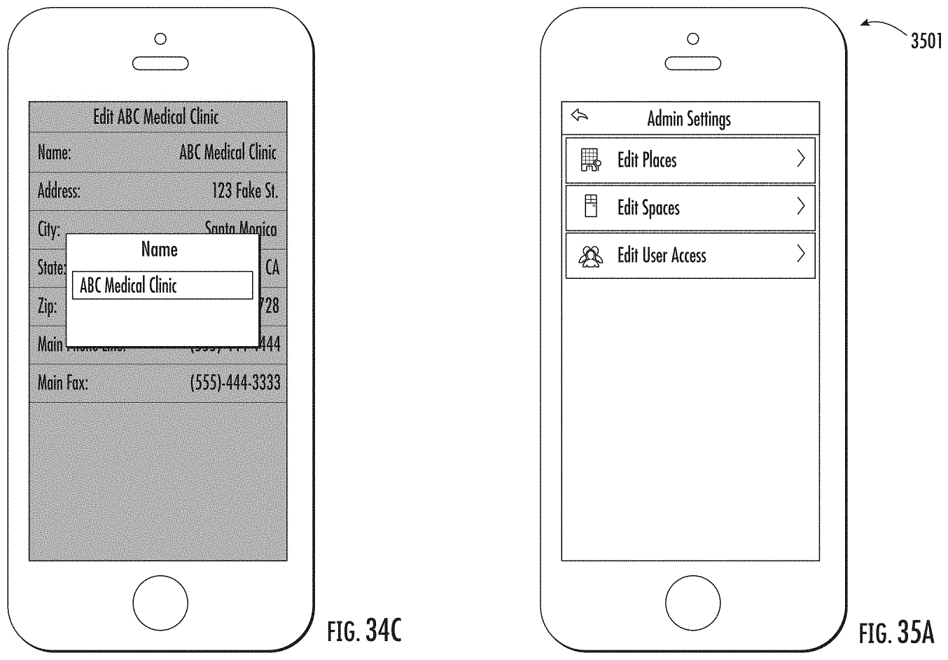

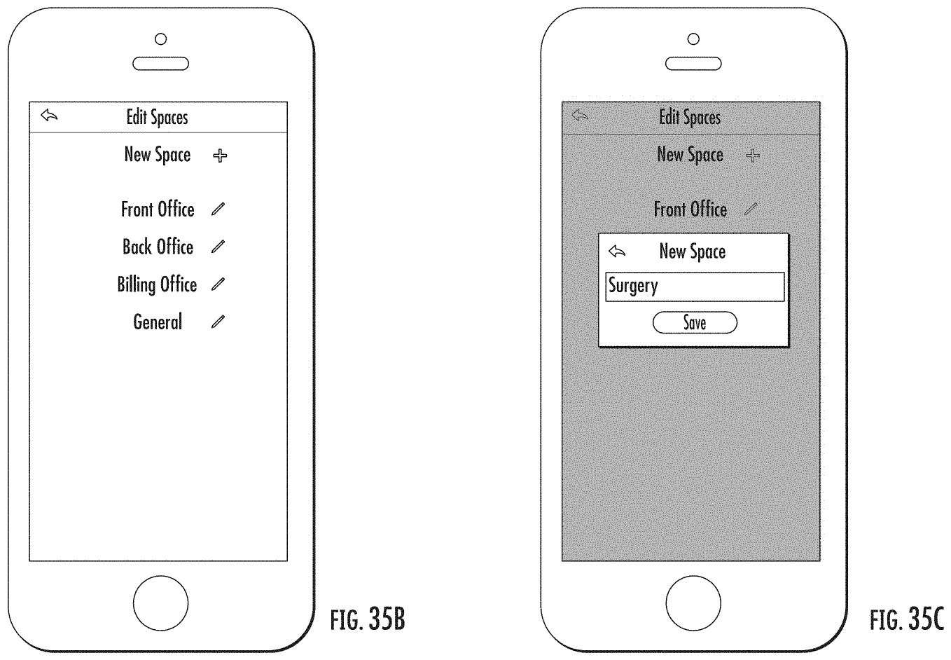

FIGS. 34A-35C show screens for editing a place as an administrator in the encrypted messaging system, as shown in 3401 and 3501.

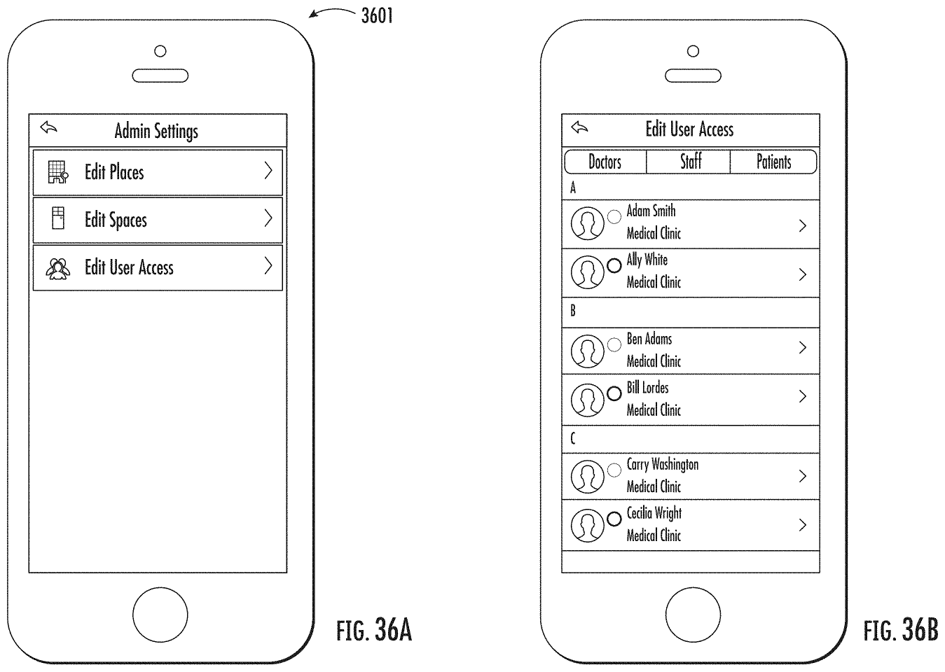

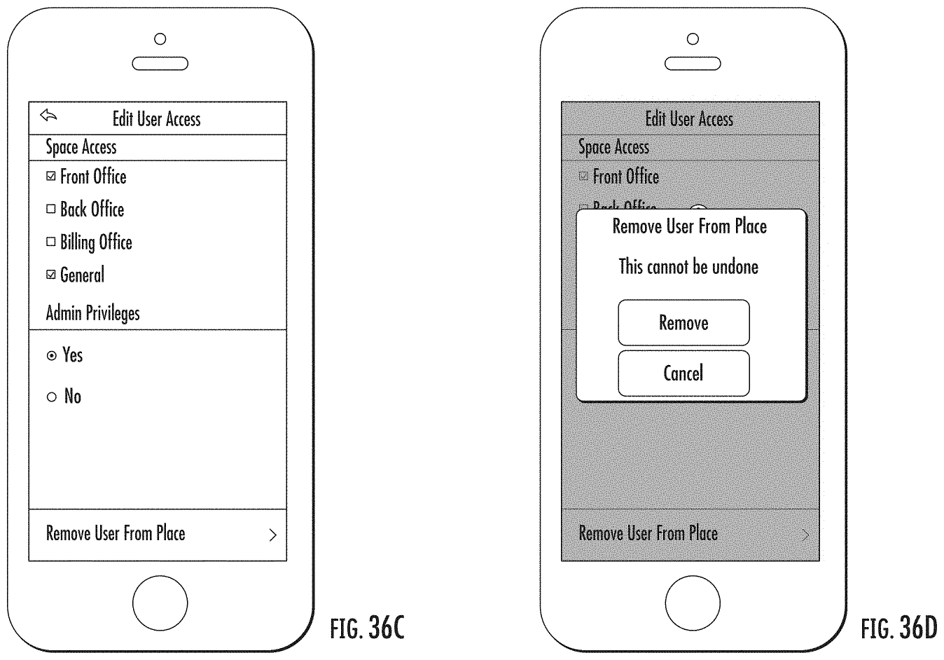

FIGS. 36A-36D show screens for an administrator to edit user access to features in the encrypted messaging system, as shown in 3601.

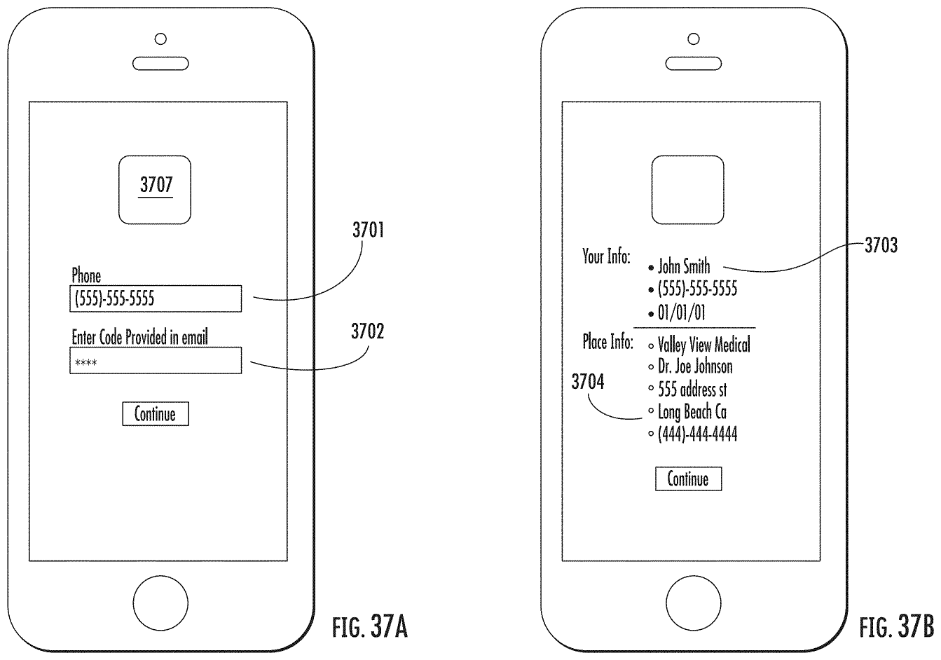

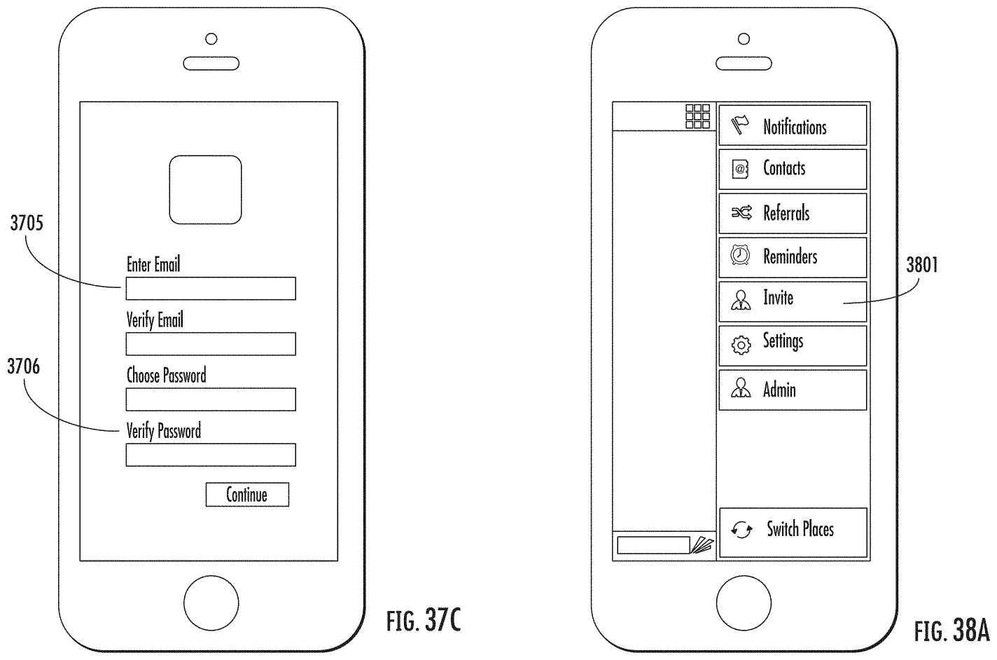

FIGS. 37A-37C show screens for registration for a patient type user. FIGS. 37A-37C include:

3701. User enters phone number

3702. User enters invite code provided by email

3703. Patient name, phone number and date invited

3704. Place/Invitee info: Place Name, Doctor Name, Places address, place phone number

3705. User enters/verifies email

3706. User enters/verifies password

3707. A logo is included here

This grants access to the new user as a patient with access to the encrypted messaging system. The user patient is added to place contact list.

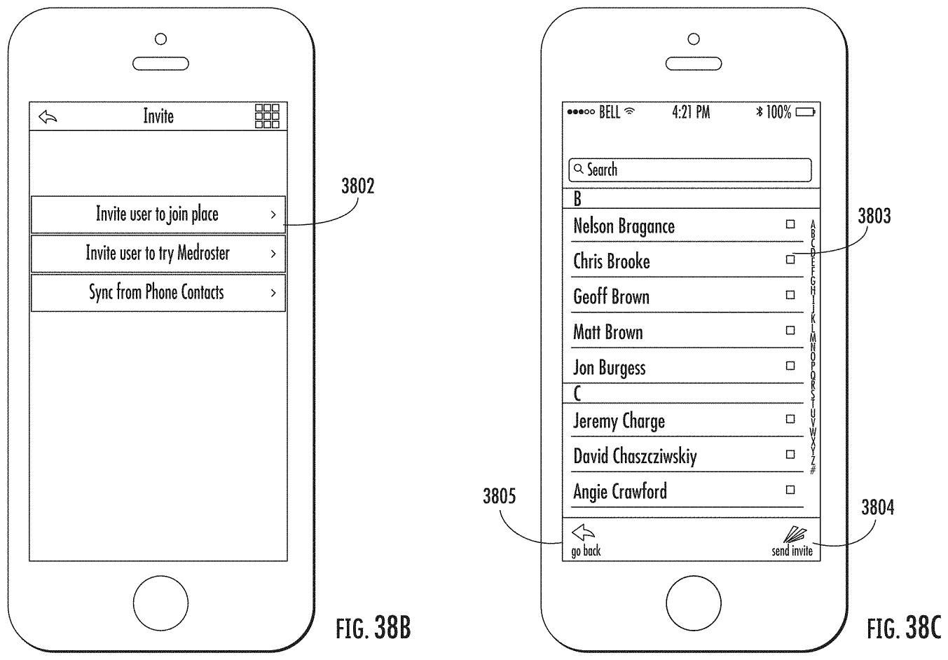

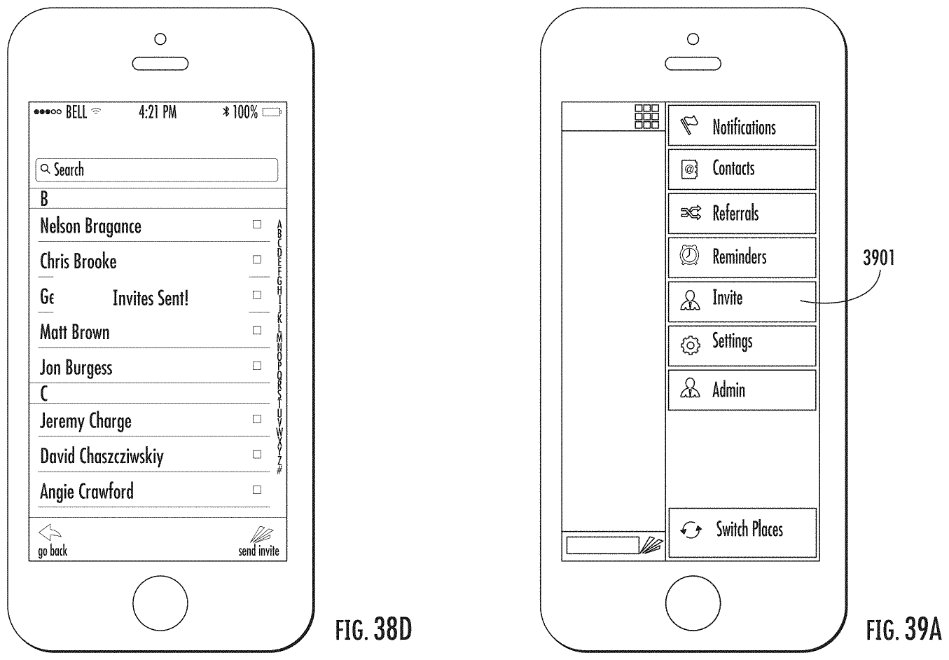

FIGS. 38A-38D show screens for a doctor or other administrator of a place to invite users. FIGS. 38A-38D include:

3801. User clicks invite button to view the second screen from the left.

3802. user clicks “invite user to join place” to access phone contact list to view the third screen from the left

3803. shows all contacts in user's phonebook with checkboxes. Checking the box may add contact to list of users to send invite to. Whether the contact is a phone number/email address may depend how the invite is sent

3804. Click to complete invitation

3805. Click to go back to the second screen from the left.

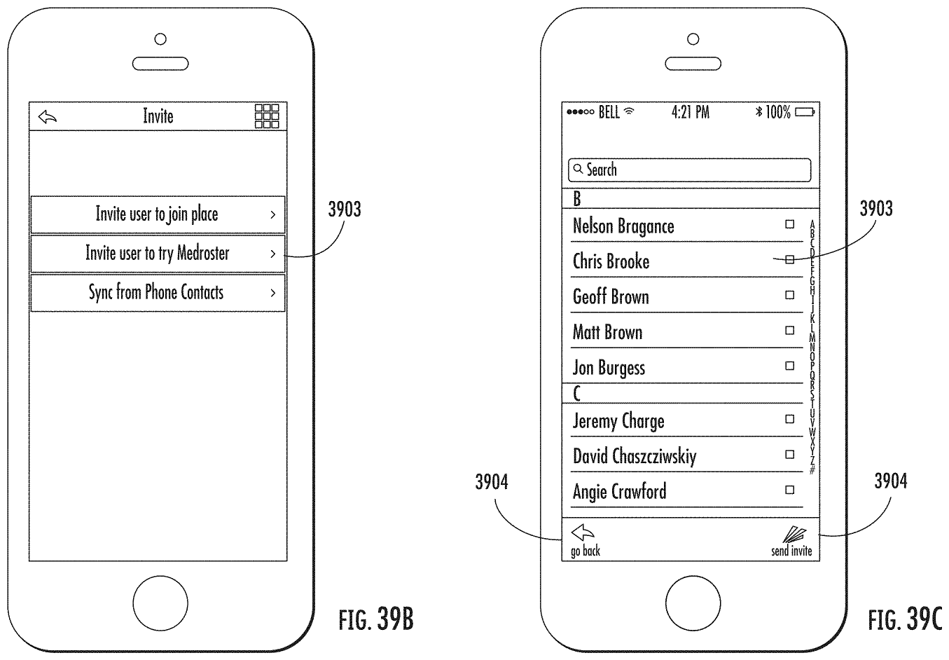

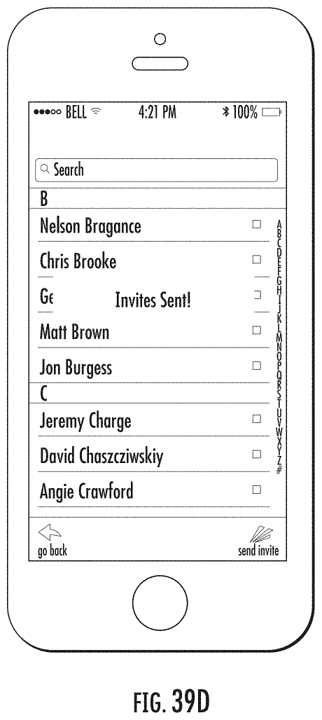

FIGS. 39A-39D show screens for a doctor or other administrator to invite users to try the encrypted messaging system. FIGS. 39A-39D include:

3901. User clicks invite button to view the second screen from the left

3902. User clicks “invite user to join place” to access phone contact list shown in the third screen from the left

3903. Shows all contacts in user's phonebook with checkboxes. Checking the box may add contact to list of users to send invite to. Whether the contact is a phone number/email address may depend how the invite is sent.

3904. Click to complete invitation.

3905. Click to go back to the second screen from the left.

An invitation to use the encrypted messaging system may be made through a text message, an email, or other suitable method to an invitee. For example, a text message may include the message: “(User First/Last name) has invited you to try Medroster.com! The best way to communicate with your colleagues and patients! Click the link to download (link)”.

Verified User

The encrypted messaging system may require multiple identity verification, before a user becomes an authenticated user and is allowed access to features of the encrypted messaging system. This means that users of the encrypted messaging system must complete two or more identity verification steps before they are authenticated as a valid user in the encrypted messaging system. Different types of users may be required to provide different verification steps. For example, the encrypted messaging system requires, before a new user is allowed to sign up with the encrypted messaging system to register as a new place, patient, or doctor.

Some examples of information that may be used to verify a new user as a doctor or a place may include a fax number, a phone number (e.g., a business phone number, a personal phone number, a mobile phone number), a National Provider Identifier (NPI) number, a physical address, or any combination of these. In an implementation, a referring user that refers the new user may also act as a verification step. Some examples of information that may be used to verify a new user as a patient may include an address, a mobile phone number, an email address, doctor provided code, or any combination of these. In an implementation, the encrypted messaging system does not require a new user registering as a patient to provide a social security number or a driver's license number as a verification step. The encrypted messaging system may have access to this information but does not require it for confidentiality purposes (e.g., patients may not provide this information to a doctor, this information is susceptible to abuse by others if accessed). The encrypted messaging system may use other information to identify a patient, such as a phone number they registered with a medical office.

The encrypted messaging system may interface with third-party databases, to collect information that may be used to verify a place or a doctor. When the new user attempts to register a new place or a new user, information from the third-party databases may be used to confirm that the new place or new user is whom they say. For example, the new user may be required to confirm information from the third-party database. The third-party database, for example, may be a state registry of registered doctors.

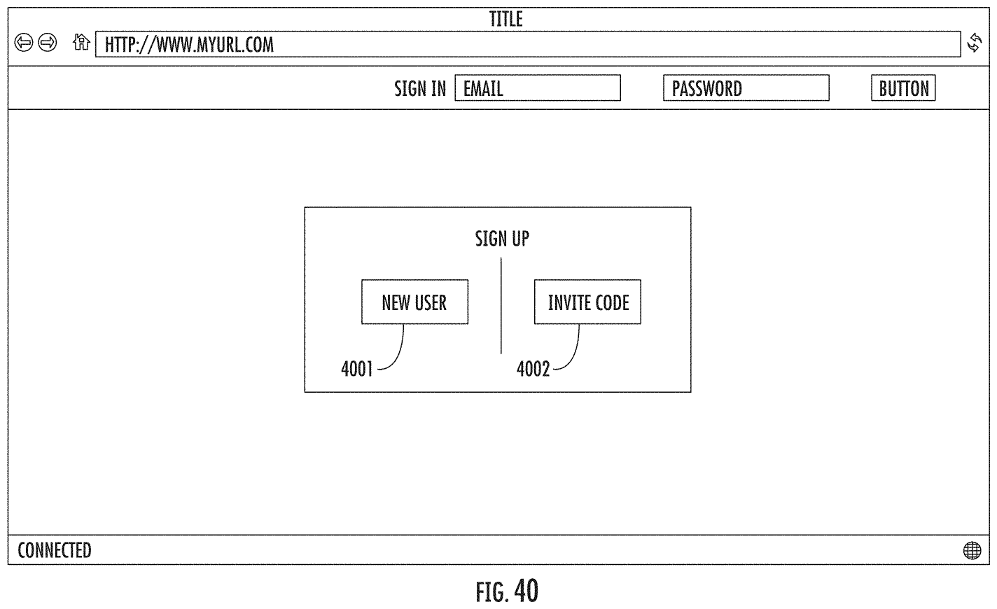

FIGS. 40-61 show screens of an encrypted messaging system. The screens include screens captured from a browser executing on a computing device (e.g., personal computer, tablet, smartphone) or an application designed to execute on a smartphone or tablet.

FIGS. 40-61 show screen guiding through a user during a signup process. The signup process is used to answer the following questions:
Was the User invited?
Is the user a Doctor or a Staff member?
Is the user in our database?
Is the Place in our Database?

FIG. 40 shows an opening screen for the signup process. FIG. 40 includes:

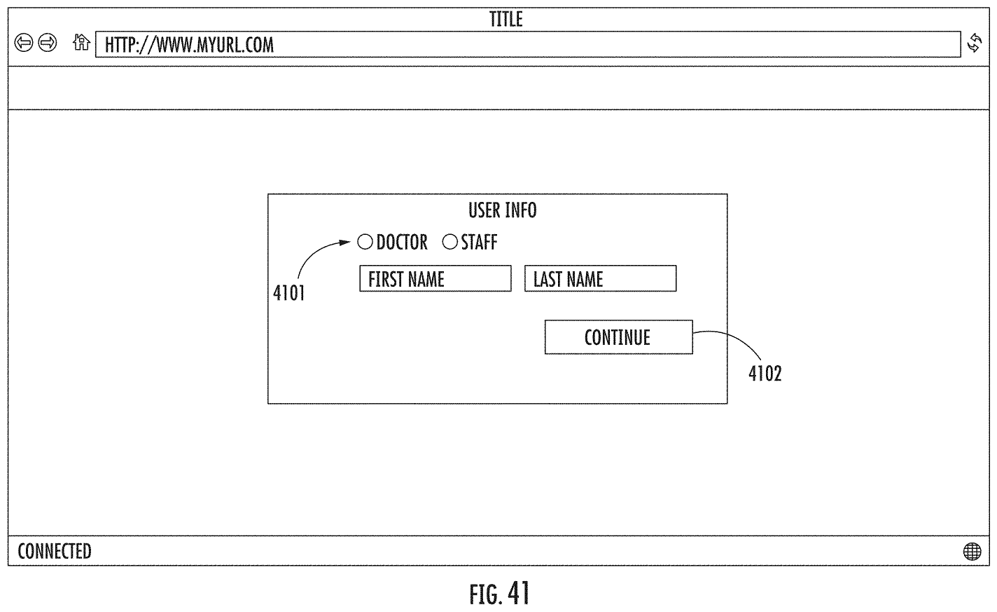

4001. If the user has not been invited—proceed to FIG. 41

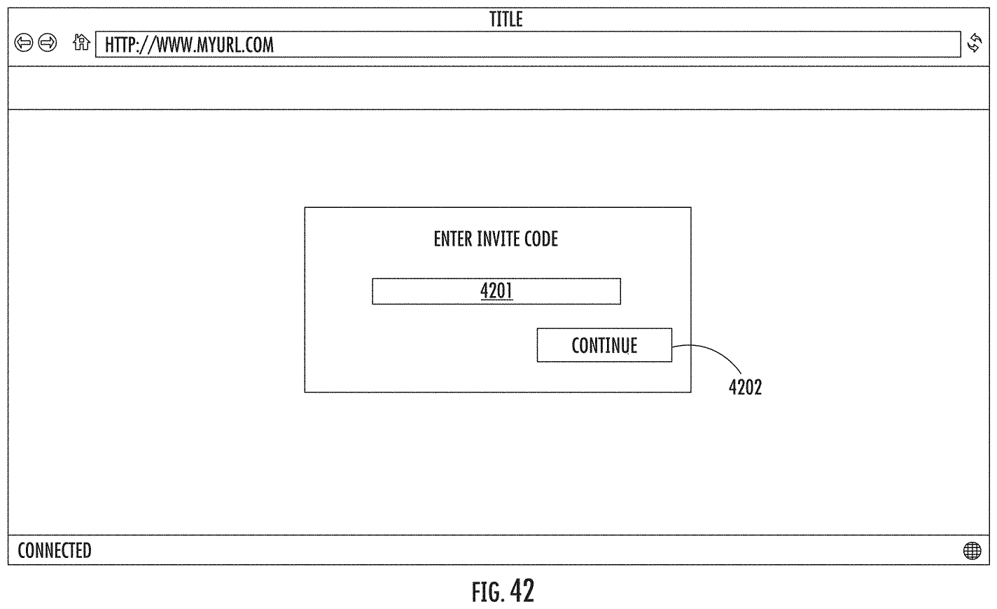

4002. If the user has received an invite code via fax/email/text—proceed to FIG. 42

FIG. 41 shows a screen to collect user information. FIG. 41 includes:

4101. To determine whether the user is eligible to create/own a place, they must be a doctor. The encrypted messaging system may use the users First/Last name to search the database for user preloaded information

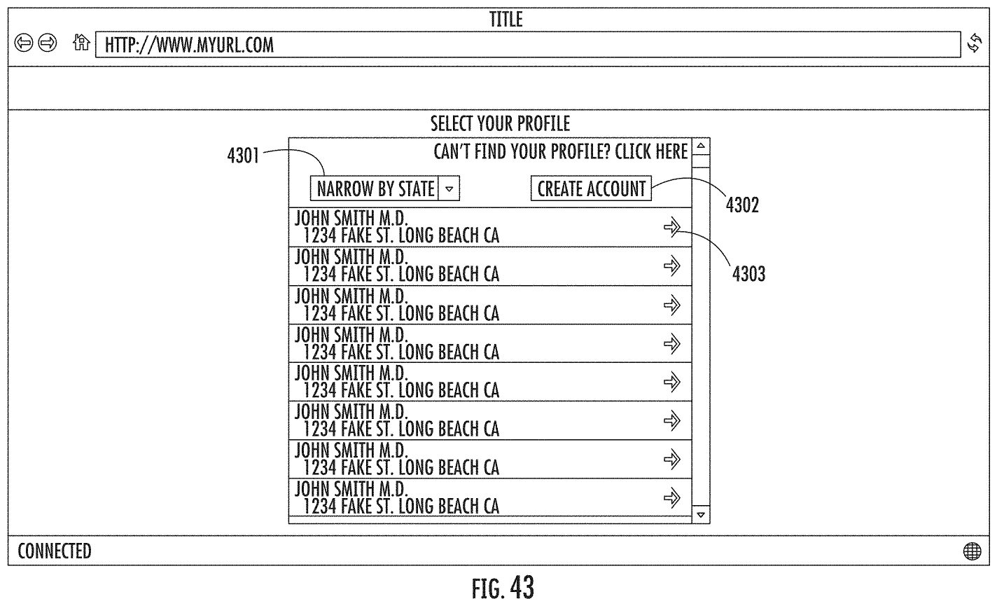

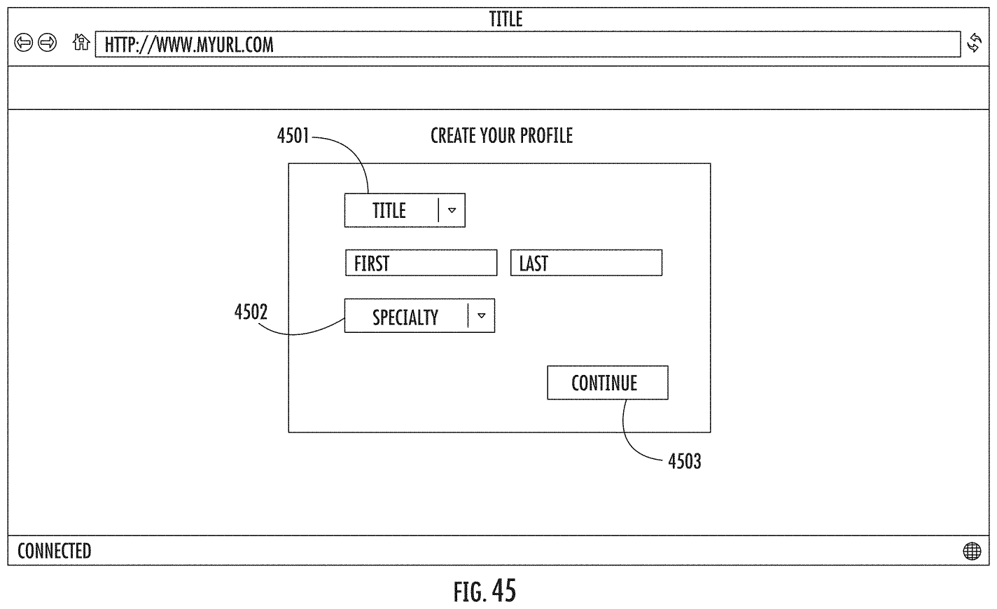

4102. If there is a user in the database with that name, they may be directed to FIG. 43. If not found, they may be directed to FIG. 45.

FIG. 42 shows a screen to input an invite code. A user with an invitation code may have received it from another user, through email, fax, or a text. This is also a way to for the users to self-verify each other. If a doctor invites someone, that means they are responsible for that person's access to the encrypted messaging system, giving a layer of security and making it HIPAA compliant. FIG. 42 includes

4201. User enters invite code here

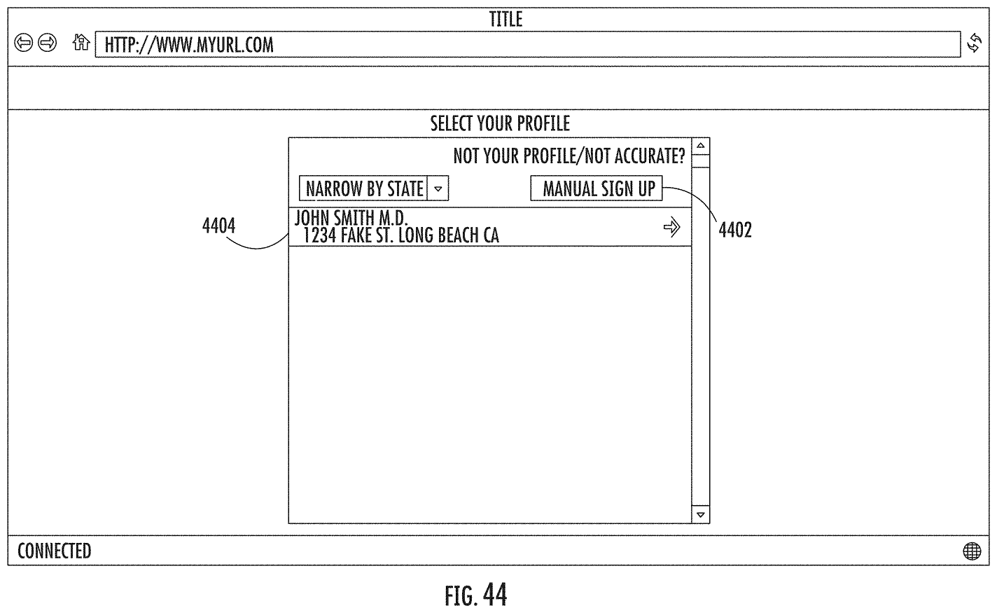

4202. If invite code is correct/valid, it depends whether the user's information is in the encrypted messaging system's database or not on where they may be directed. If they are in the database, they may be directed to FIG. 44 to verify that their info is correct. If they are not found, they may be taken to FIG. 45 to complete their full profile.

FIG. 43 shows a screen to choose a particular identity found in a listing of medical providers. For example, if the user's first and last name match up to users in the database, it is assumed, unless the name is very uncommon, that there may be multiple users with the same First and Last name. In this instance, the user may choose from a list of First/Last names in the database along with their respective business addresses. The users may use their address to narrow down the list and find themselves. FIG. 43 includes:

4301. If the user needs to narrow the list down, they can use this dropdown to select their specific state

4302. If the user searches through the list and cannot find themselves in our database, they may choose “create account” and be directed to FIG. 45.

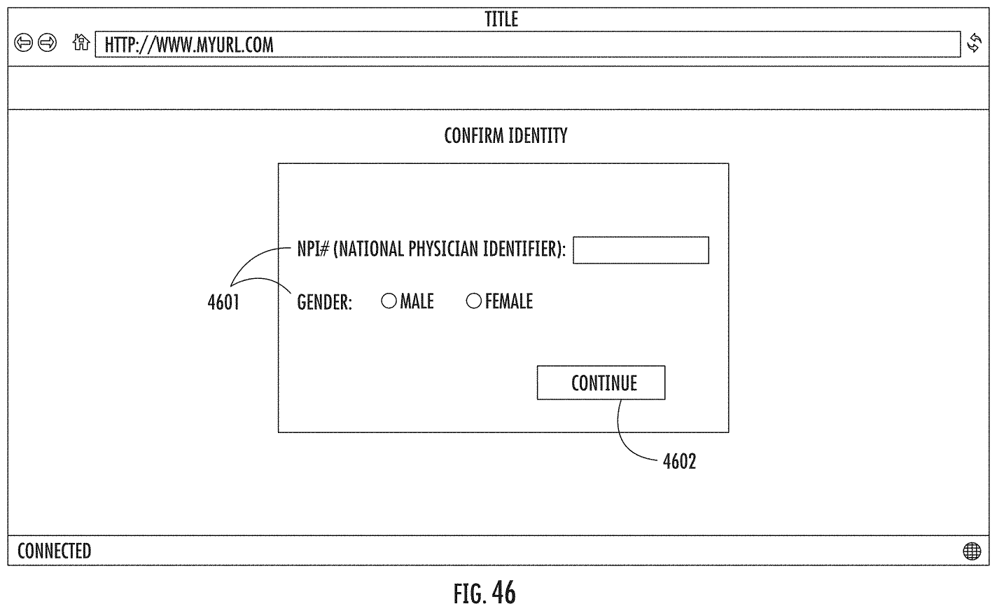

4303. When/if the user finds the correct database info for themselves, they may select themselves and be directed to FIG. 46 to confirm their identity. If no database information can be found, the user may be directed to FIG. 45.

FIG. 44 shows a screen to verify database information. In the case that a user is invited and their information happens to be in our database, they may be shown the data that the encrypted messaging system may already have for them. FIG. 44 includes:

4402. If the user does not recognize the information displayed as their own, they are given the option to create their own account manually, such as shown in FIG. 45.

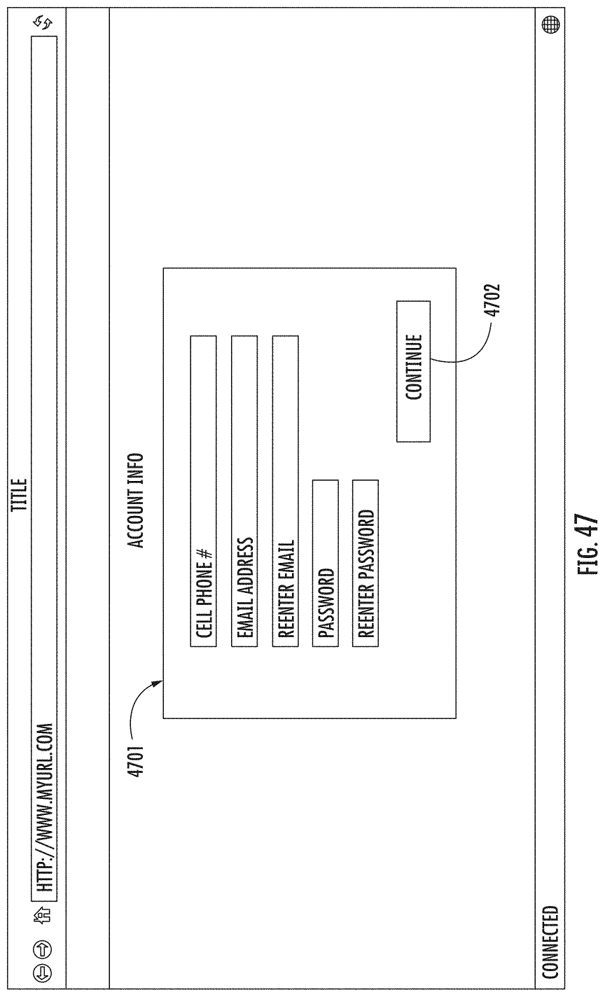

4404. If the user's info is correct, there are two possible instances:
The first case is that the user belongs to a PLACE that has already been established by another user. In that case, the invited user may skip the “Place Search” process and be directed to the screen of FIG. 47 to complete their ACCOUNT INFO. This may be used most frequently by the STAFF of a place, seeing as they may be invited by doctors
The second case is that this user may have been invited by a user that does not belong to the same place as the 1st user. In this case, the user may search for his respective place, being directed to 5 to complete account info, and then to place search.

FIG. 45 shows a screen to complete a user profile. If the user's information is not correct/cannot be found, they may be directed to this screen. FIG. 45 includes:

4501. complete list of medical titles. For example, the encrypted messaging system may interface with third-party databases to determine any titles associated with a physician, such as the one available at http://www.pamforg/physicians/titles.html. If the user already filled in information (e.g., first name) in a previous screen, these fields should contain what the user provided previously

4502. complete list of medical specialties. For example, the encrypted messaging system may interface with third-party databases to determine any specialties associated with a physician, such as the one available at http://www.mclarenhealthplan.org/HealthAdvantage/AListofDefinitionsofMedicalSpecialties Adv.aspx

4503. This may take the user to complete their account info screen as shown in FIG. 46. These fields are shown for both doctors & staff members.

FIG. 46 shows a screen to confirm an identity. To verify the user's identity, the encrypted messaging system may collect their NPI # and their gender 4601. If both points of data match our database, when the user presses “continue” 4602, the user may be directed to the screen of FIG. 47.

FIG. 47 shows a screen to complete an account. FIG. 47 includes:

4701. All fields are required fields

4702. if all info is collected, emails and passwords match, the user may be directed to search for their place. If they have been invited, the user may be taken to their dashboard. If the user is new and needs their place verified, or their account verified, they may be put on an administrative hold to have their account verified.

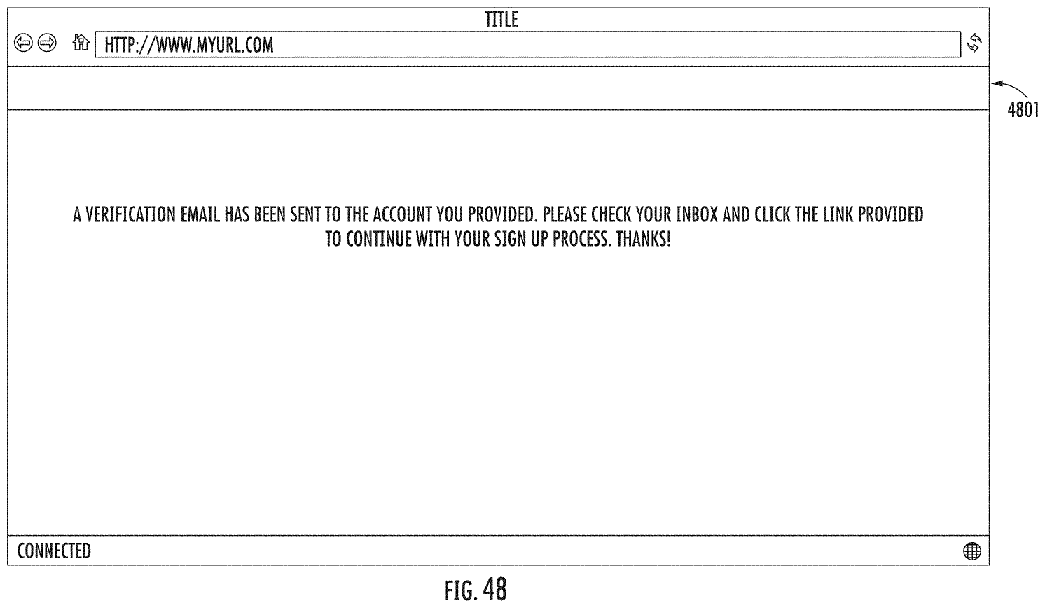

FIG. 48 shows a screen for email verification. FIG. 48 includes:

4801. After a user enters their account info, they may view this screen. At the same time, an email (example email shown in FIG. 50) may be sent to the email address the user provided. The email may contain a link to verify the account.

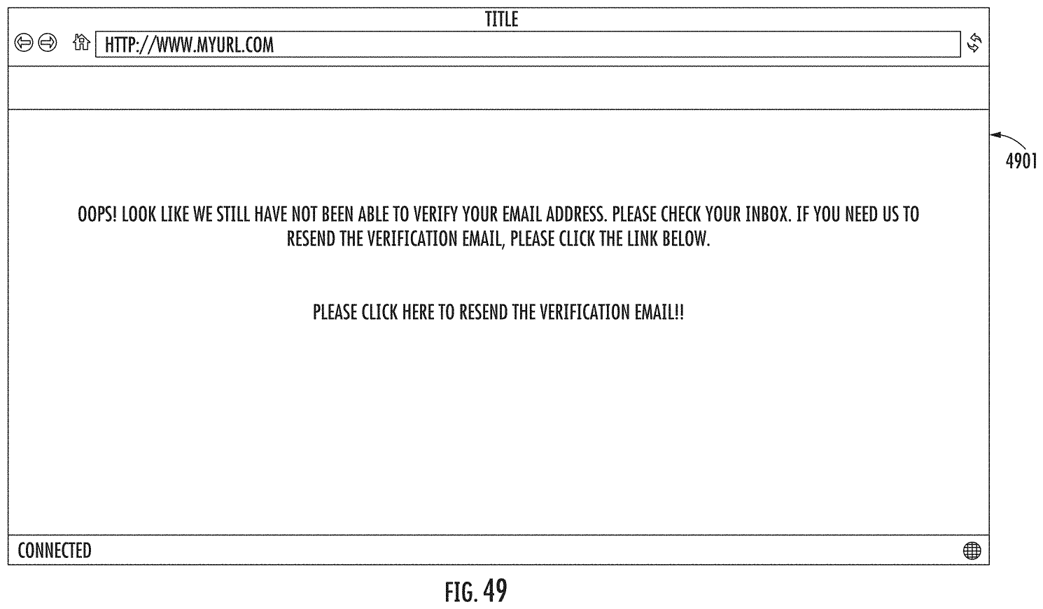

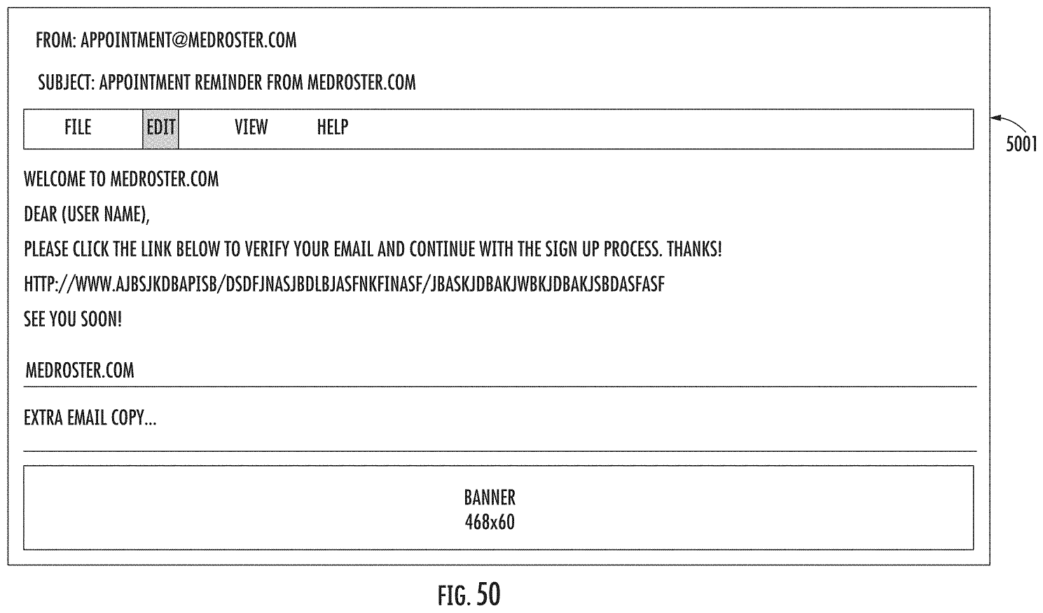

FIG. 49 shows a screen when a login is attempted before verification, as shown in 4901. If the user attempts to login before they have verified their account via email, they may see this screen. If the user has not received the email, they may have the option to click the link to resend the email.

FIG. 50 shows an email to verify an account as shown in 5001. This is the email the user may receive to verify their account. The same email may be used if they choose to resend their verification email as shown in FIG. 49, If they click the link, they may be directed to the screen shown in FIG. 49, which gives confirmation of their email being verified.

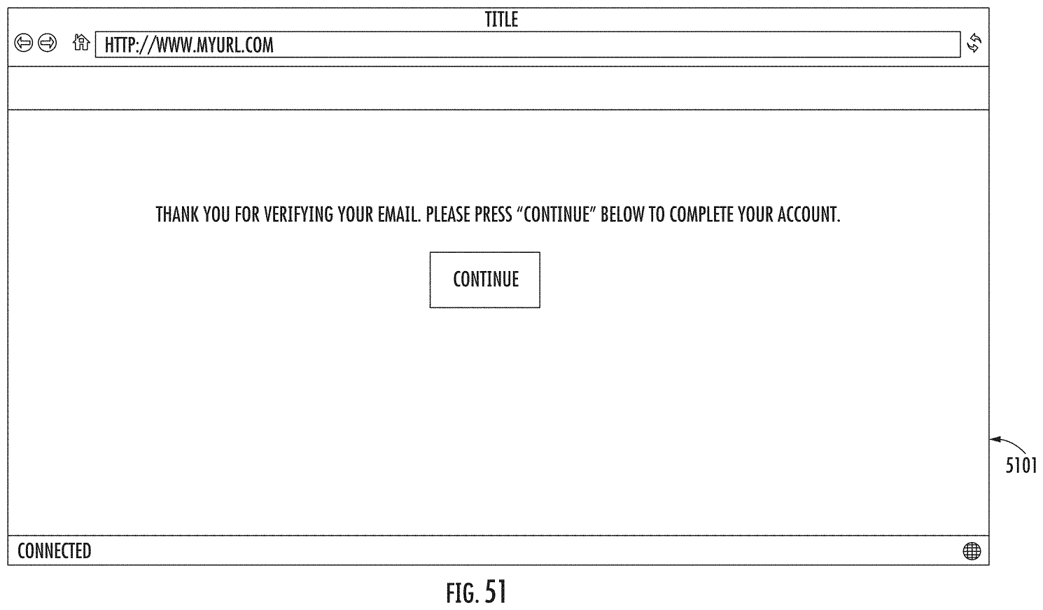

FIG. 51 shows a screen that email verification is complete as shown in 5101. After the user clicks the link in the verification email, they may see this screen. Pressing the “continue” button may take the user to the next screen in their sign-up process.

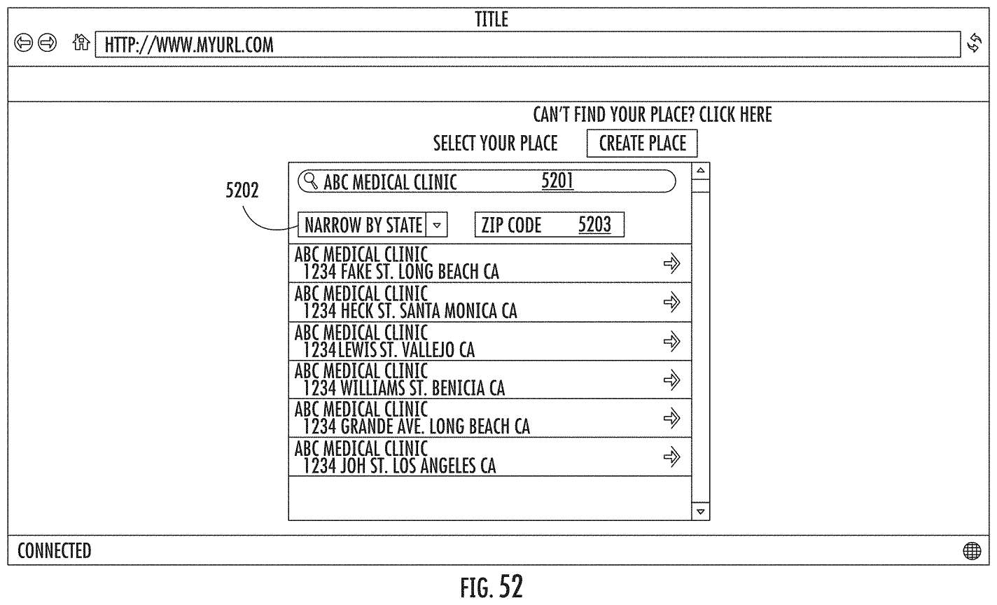

FIG. 52 shows a screen to perform a place search. Users may search for their place in the search bar (5201) and if the list of possible places is too long, they can either narrow by state (5202) or by zip code (5203). There may be instances where a user cannot find their respective place through our database search, but their place has already been established. In this case, the encrypted messaging system may have to figure out some other way to verify the place they belong to (discuss). If their place is not in our database, they have the option to create a place, but further verification may be needed.

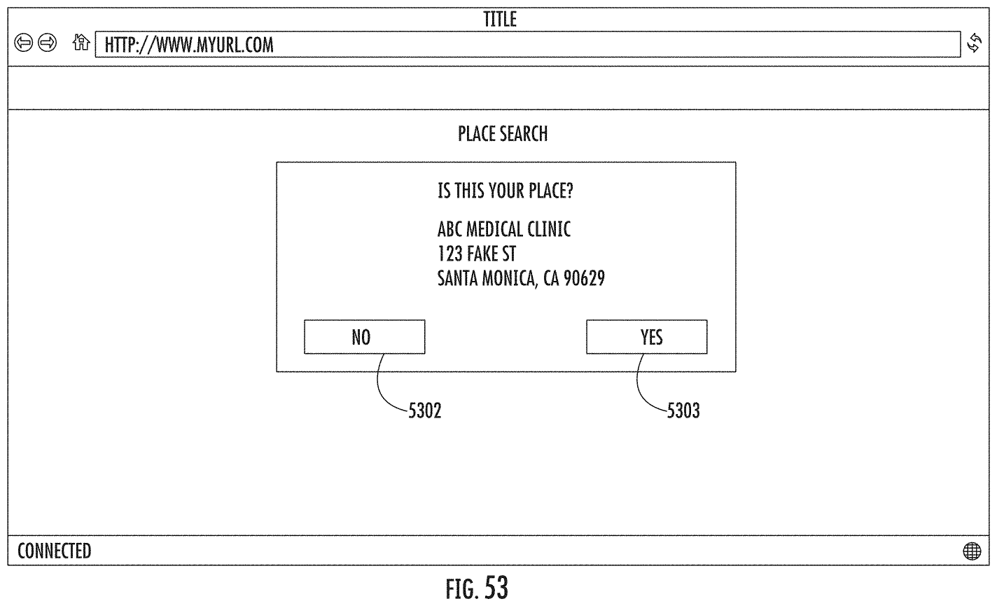

FIG. 53 shows a screen to confirm a place. When/if the user finds their correct place, they may be directed to this screen to verify that this is the correct information. FIG. 53 includes:

5302. If information is incorrect, the user may select no, and be directed to create their place. As stated previously, in the instance that the user cannot find their correct place, but it does exist, the encrypted messaging system may need to figure out a way to avoid instances of duplicate places being created.

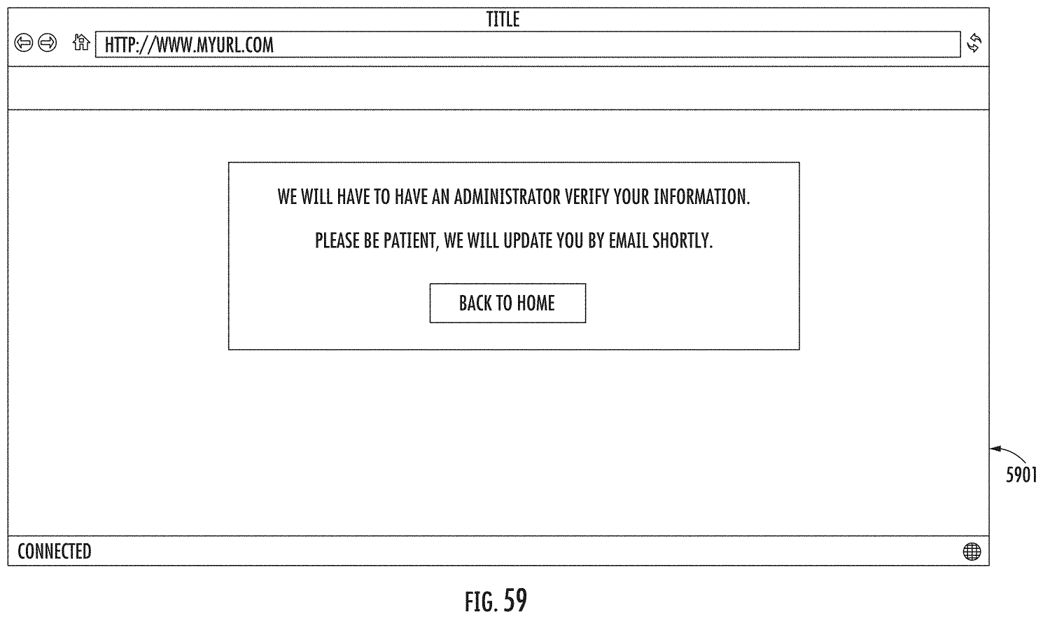

5303. If the Place info is correct and this is the first user to sign up for this place, the user may be directed to verify themselves. If not the first user to sign up for this place, they may be redirected to a screen such as FIG. 59 so the Place administrator may verify them.

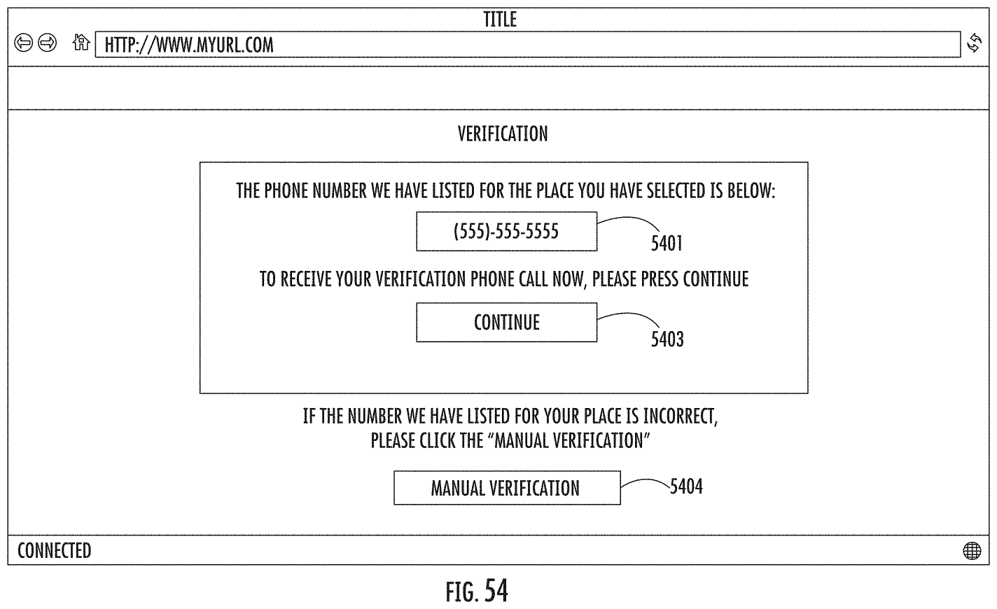

FIG. 54 shows a screen for phone verification. If the encrypted messaging system has been unable to verify a user, the encrypted messaging system uses the main phone number the encrypted messaging system has in the database, once the user selects “continue” our system may call the user and provide a verification code. FIG. 54 includes:

5401. The user's main phone number from the database

5403. Pressing “continue” may prompt the system to call the phone number and move to the next screen

5404. Pressing “manual verification” may take the user to view screen shown in FIG. 59 and send the user information to the “admin verification” screen.

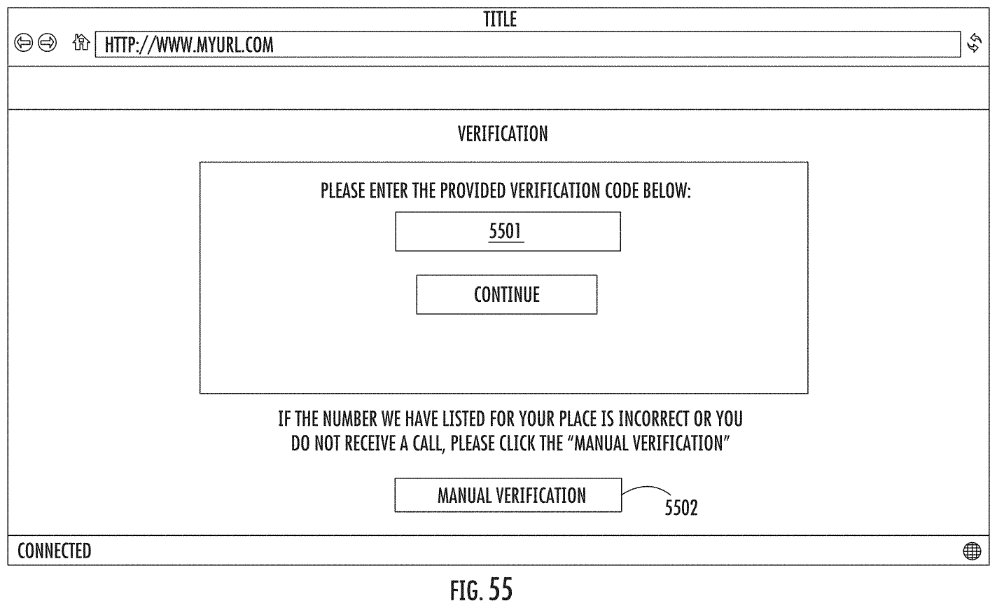

FIG. 55 shows an additional screen for phone verification. For example, after the user prompts the system to call the database provided phone number, the user may receive a call to that number. The system may provide the user a code to enter into the text field. FIG. 55 includes:

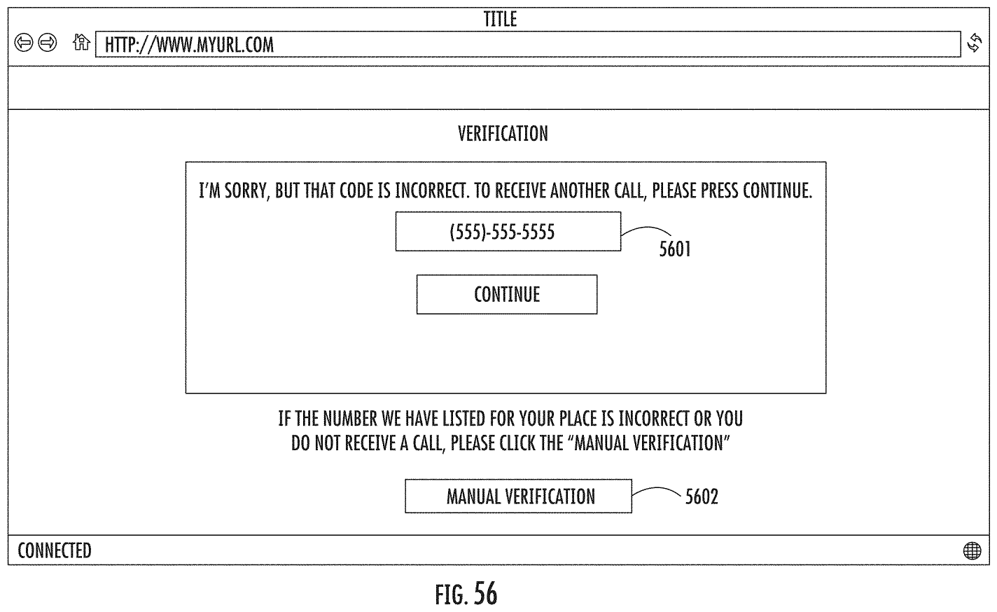

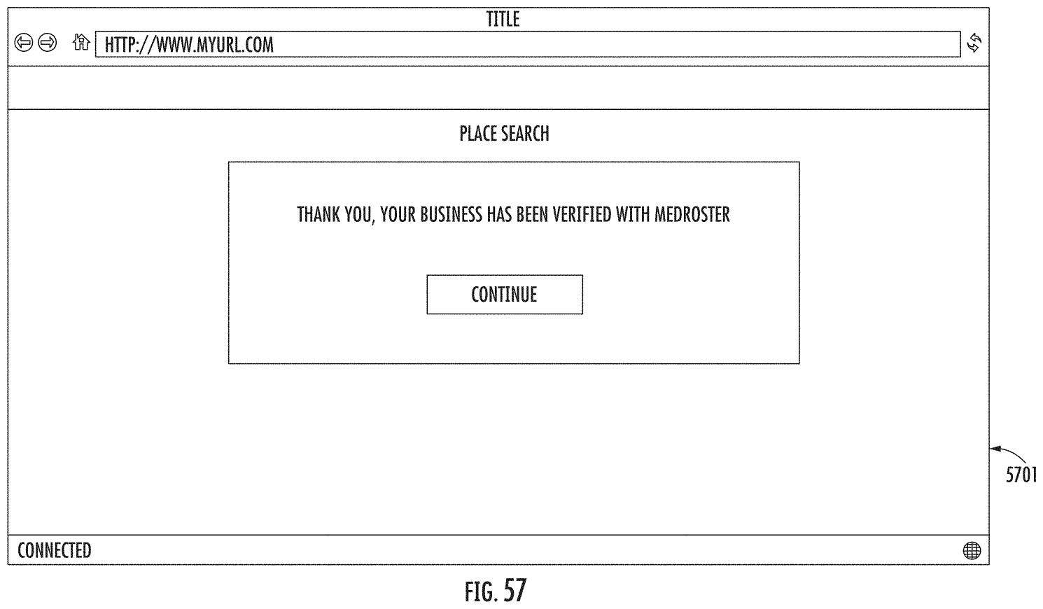

5501. Text field for verification code. Pressing continue with the correct verification code may take the user to screen shown in FIG. 57. An incorrect code may take the user to screen shown in FIG. 56.

5502. Pressing “manual verification” may take the users data to the Admin verification screen.

FIG. 56 shows a screen when a phone verification error has occurred. If the user enters an incorrect code, they may view this screen. FIG. 56 includes:

5601. The user has the option to either receive another call or manually verify his information. This cycle may continue until the user enters the correct verification code. Pressing continue may send the user to the screen shown in FIG. 54 to resend the call

5602. Manual verification.

FIG. 57 shows a screen where verification has completed, as shown in 5701. If the user calls from the correct phone number and press continue, they may come to this screen. Pressing continue may take them to the dashboard. If a staff member registers and finds their place, but their place is not active in the encrypted messaging system, they are shown a message asking them to require a doctor to sign up and activate the place. This may be similar to the screen shown in FIG. 59 (diagram 4g1—same as 4g with a different message).

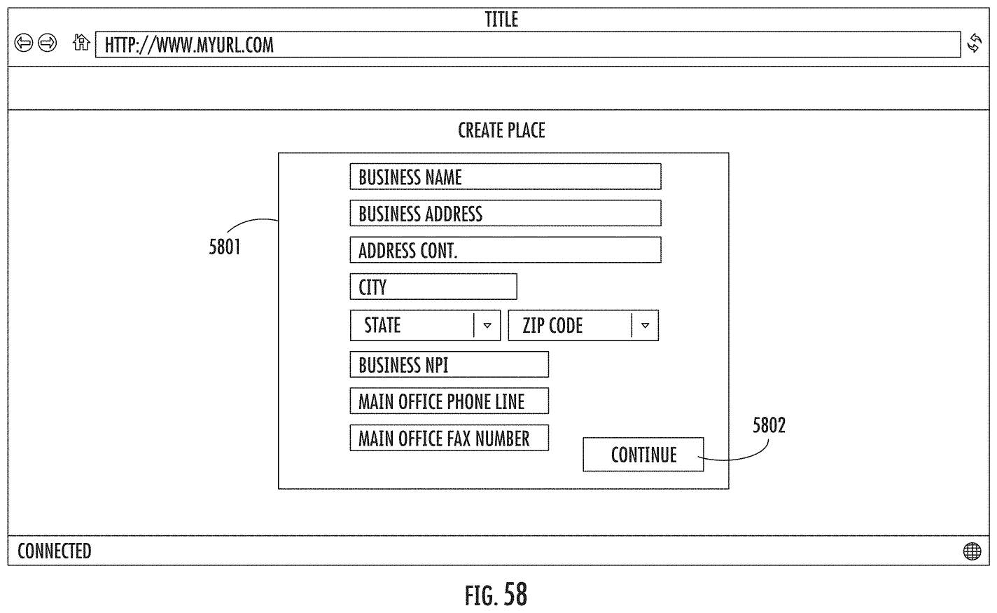

FIG. 58 shows a screen to create a place. FIG. 58 includes:

5801. In certain cases (about ten-percent of the time) a place may not be in the database, or that information may not be accurate for one reason or another. If this is the case, the user may be directed to this screen to create their place profile. This user must be a doctor, have his info verified through our database, and be in charge of this place (HIPAA) to establish this place. If the doctor user cannot be verified through our system, the user may not be allowed to create a place (HIPAA). Staff cannot create places (HIPAA)

5802. When complete, and only if all info is complete, and the user was verified through our database, the user may be directed to wait for an administrator through the encrypted messaging system to verify their account. They may be on admin hold such as the screen shown in FIG. 59.

FIG. 59 shows a screen for administrator verification, as shown in 5901. When a user attempts to join an already established place, they may need to be verified by that Place's account administrator. When a user reaches this stage, they cannot communicate with other users, view the users or spaces of the place, only the name, used in the encrypted messaging system for the place due to HIPAA violation concerns. The user, when clicking “back to home” may be allowed to view a short tutorial of the encrypted messaging system and view the dashboard.

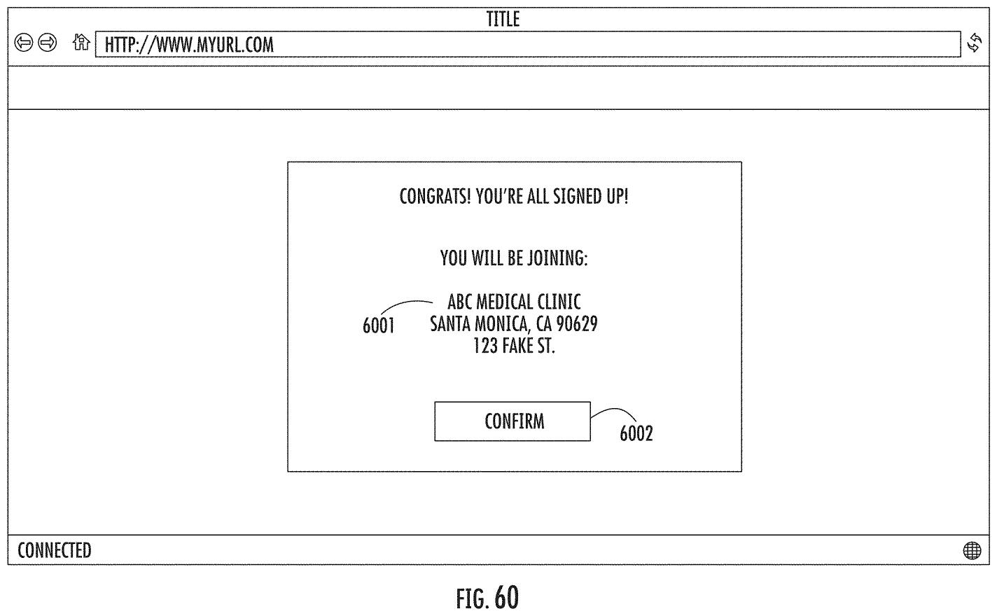

FIG. 60 shows a screen for inviting others to join a place. When a user is invited to join a place, they may have already confirmed their place by clicking the link in the email. When they complete account info, they may view this page which shows they have complete sign up, the place they join, and they may have access to the dashboard. FIG. 60 includes:

6001. Place the user was invited to

6002. Confirm button goes to dashboard, giving user access to the place.

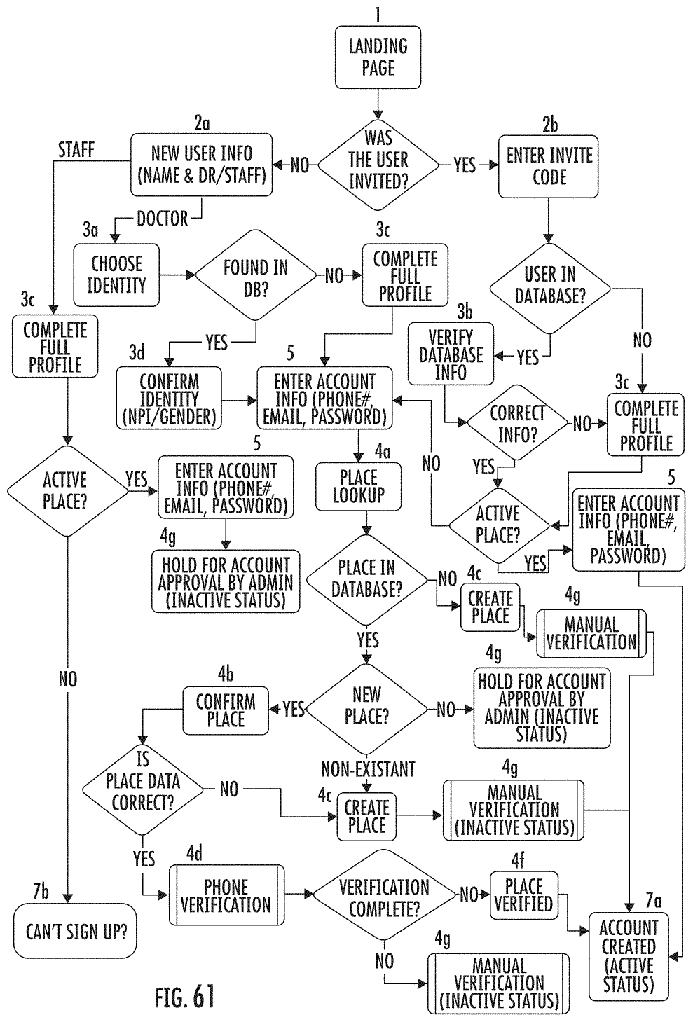

FIG. 61 shows a flow for a website that implements features of the encrypted messaging system. The flow provides a flow for each user-case. Each diagram label corresponds to the flowchart and a specific diagram identified in the FIGS. 40-60. Definitions for some of the terms used in the flow include:

Non-existent Place: A Place that is not in our database. User must create.

New Place: A place which has yet to be active in the encrypted messaging system but is in the database

Active Place/Existing Place: A place in which users are actively communicating

Non-Invited User: User signing up for medroster.com without an invite code

Inactive Status: User can access dashboard and go through the tutorial but does not have access to communications with other place users.

Table 2 provided below identifies use cases for the encrypted messaging system on a right row and a list of diagram identifiers for a flow that the particular use case would use. The diagram identifiers are included as headers for FIGS. 40-60.

TABLE 2

| User Case | Diagram Identifier |
|---|---|
| Non-invited user/Doctor/In System/New Place | 1-2a-3a-3d-5-5a-5c-5d 4a-4b-4d-7a |
| Non-invited user/Doctor/In System/Existing Place | 1-2a-3a-3d-5-5a-5c-5d 4a-4g |
| Non-invited user/Doctor/In System/Non-existing | 1-2a-3c-5-5a-5c-5d 4a-4c-4g (encrypted |
| Place | messaging system admin) |
| Non-invited user/Doctor/Not In System/Existing | 1-2a-3c-5-5a-5c-5d 4a-4b-4g |
| Place | |
| Non-invited user/Doctor/Not In System/New Place | 1-2a-3c-5-5a-5c-5d 4a-4b-4d-7a |
| Non-invited user/Staff/not in system/Existing Place | 1-2a-3c-5-5a-5c-5d 4a-4g |
| Non-Invited User/Staff/Not in System/New Place | 1-2a-7b |
| Invited User/Doctor/In System/Existing Place | 1-2b-3b-5-5a-5c-5d 7a |
| Invited User/Doctor/Not in System/Existing Place | 1-2b-3c-5-5a-5c-5d 7a |
| Invited User/Doctor/Not In System/New Place | 1-2b-3c-5-5a-5c-5d 4a-4b-4d-7a |
| Invited User/Staff/Not in system/Existing Place | 1-2b-3c-5-5a-5c-5d-7a |
| Referral - Non-invited user/Doctor/In System/New | 1-2b-3b-5-5a-5c-5d-7a |
| Place | |
| Referral - Non-invited user/Doctor/In | 1-2b-3c-5-5a-5c-5d-7a |
| System/Existing Place | |
| Referral - Non-invited user/Doctor/Not In | 1-2b-3bc-5-5a-5c-5d-4a-4b-4d-7a |
| System/Existing Place | |
| Referral - Non-invited user/Doctor/Not In | 1-2b-3c-5-5a-5c-5d-7a |
| System/New Place | |

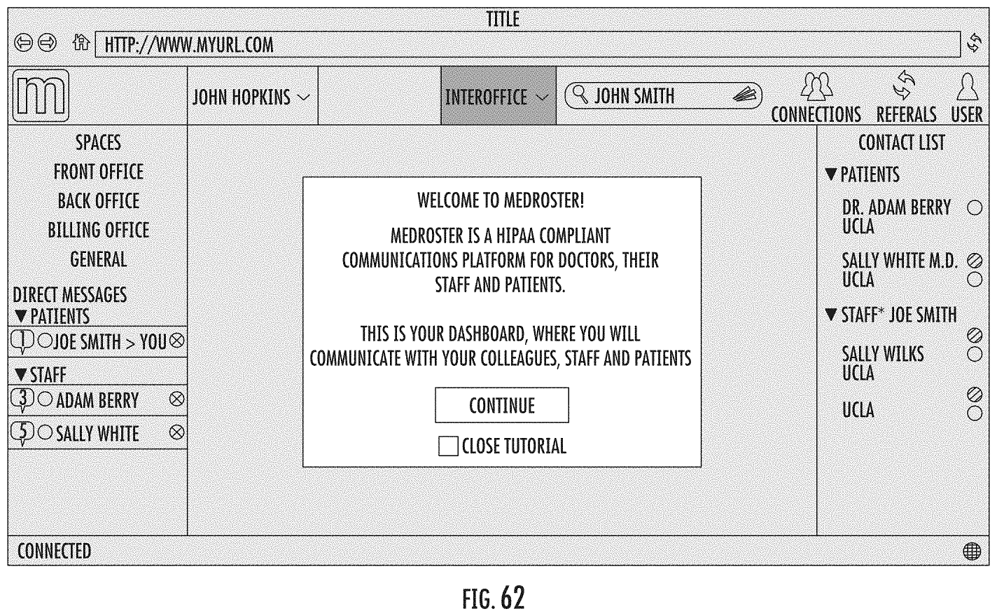

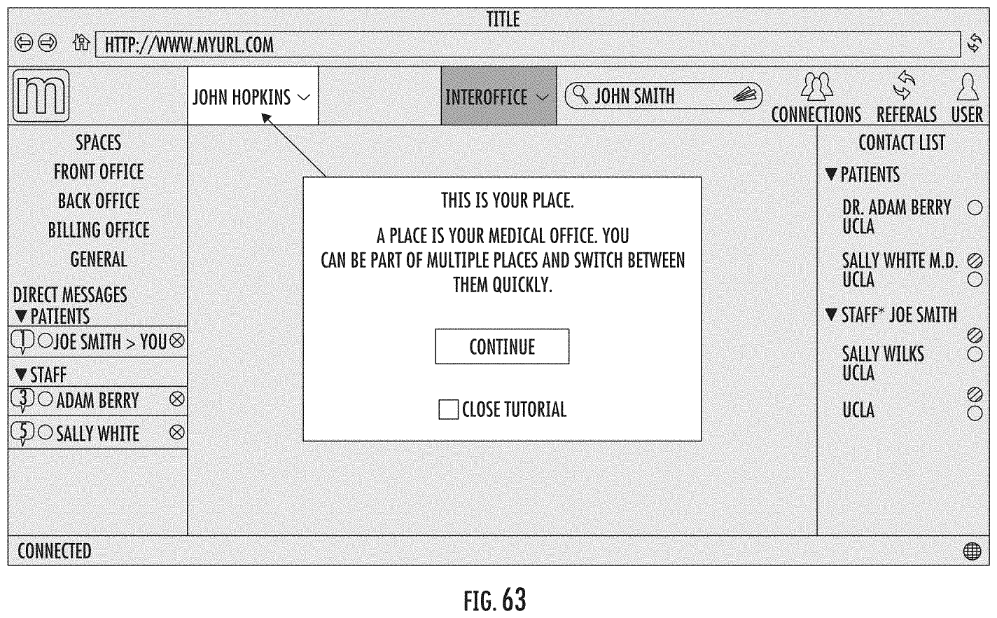

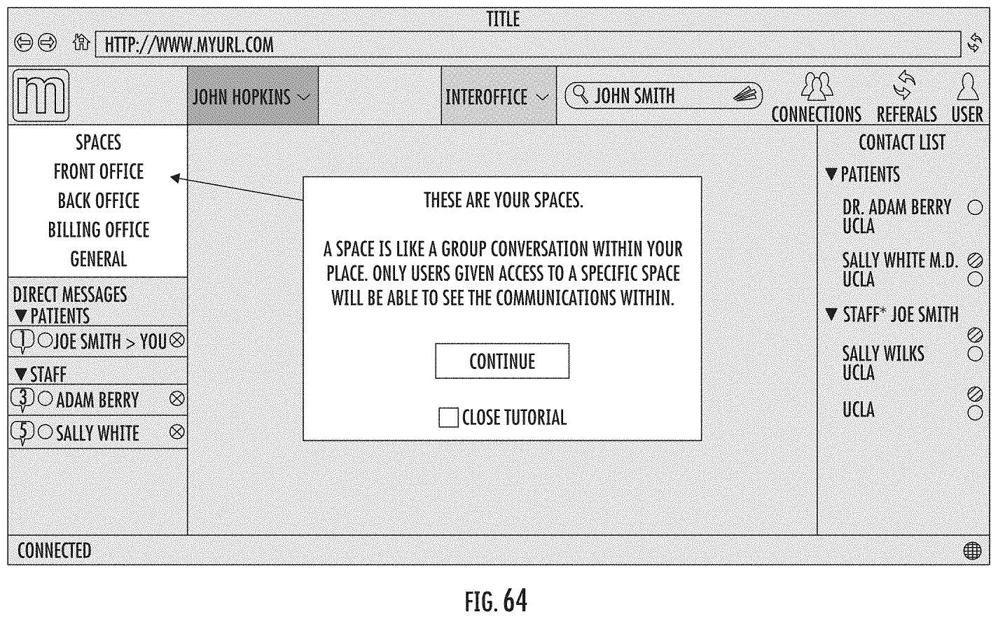

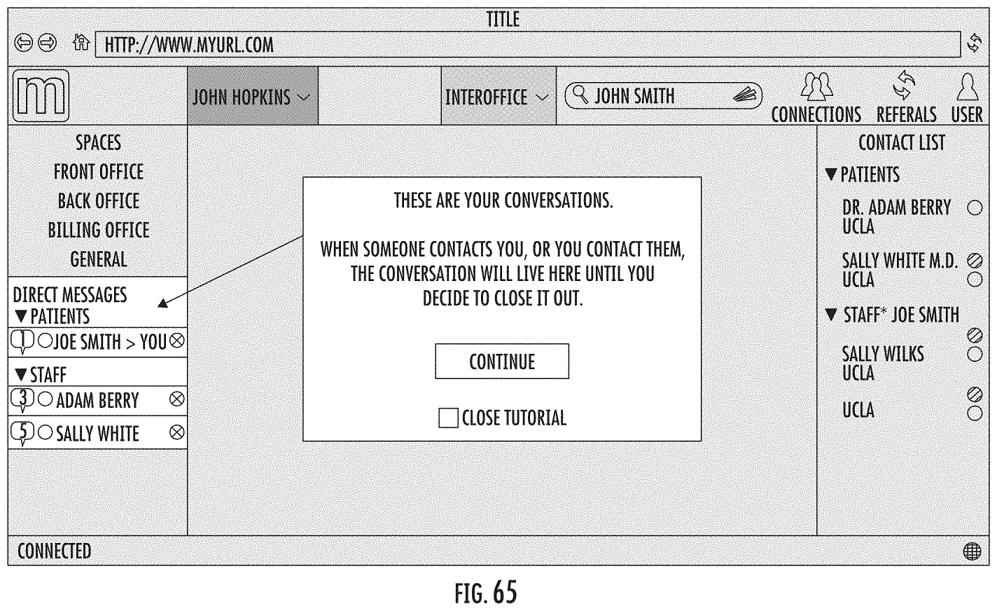

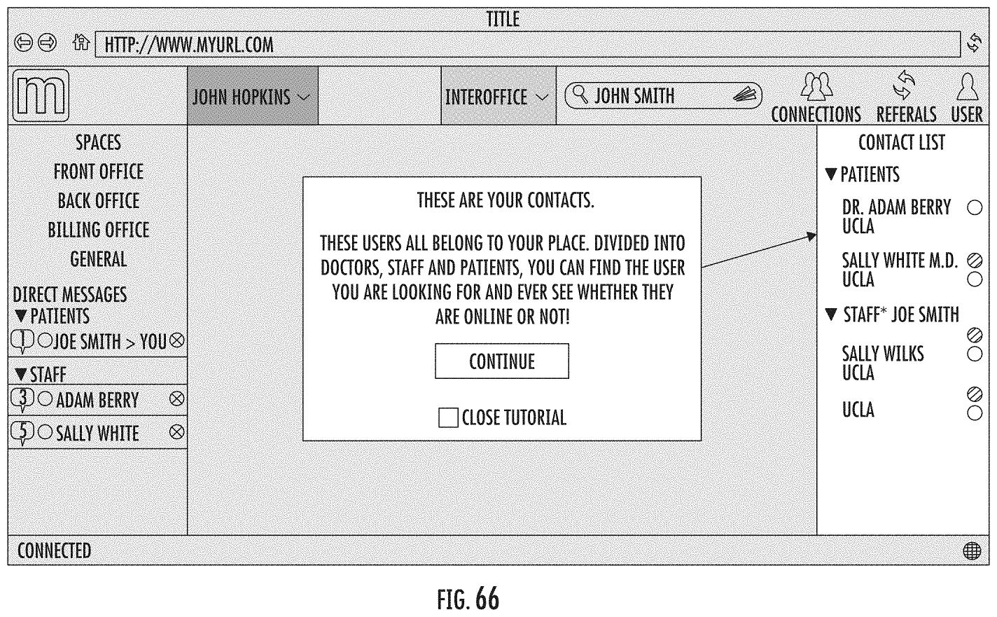

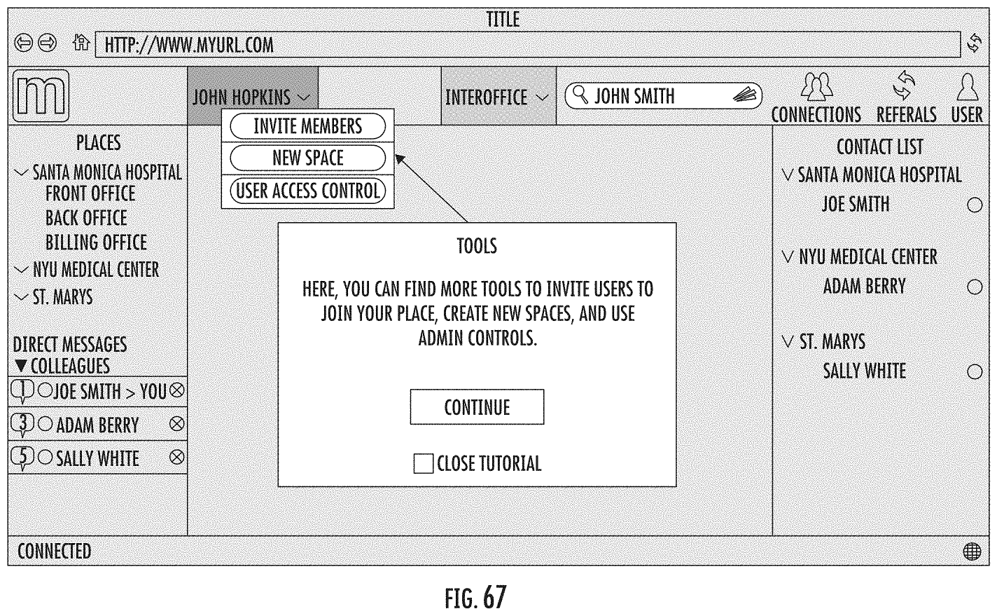

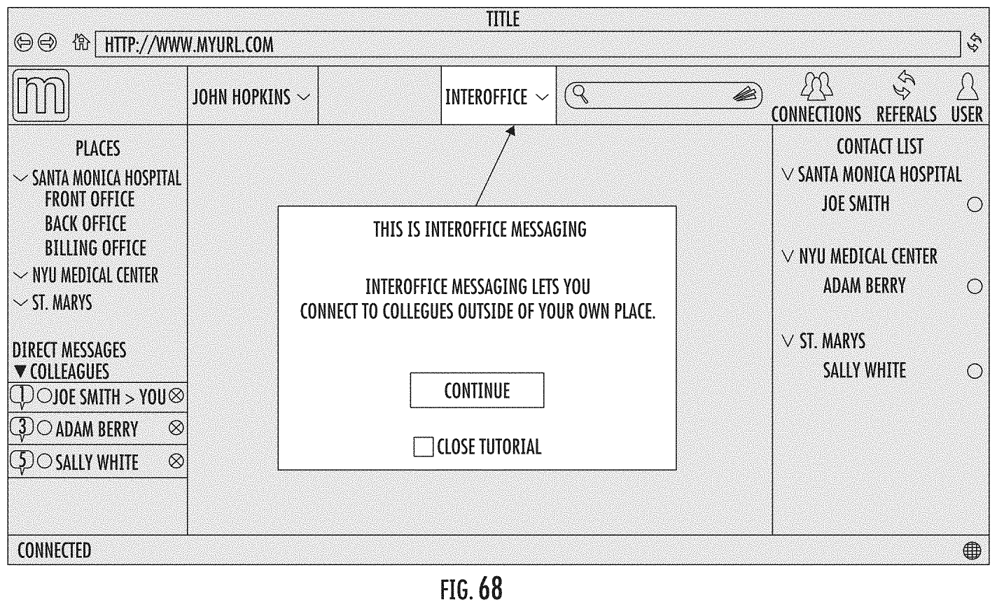

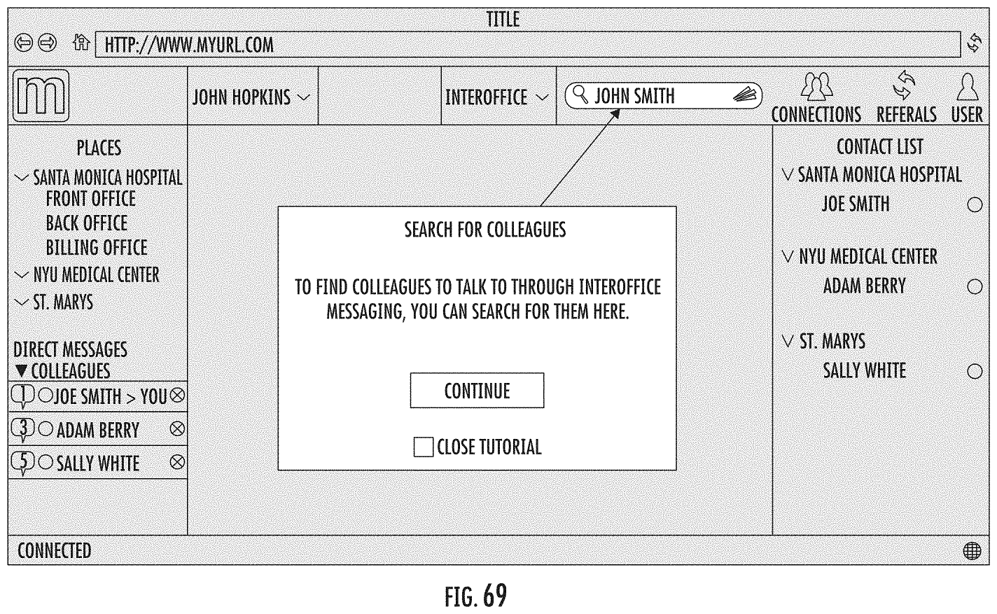

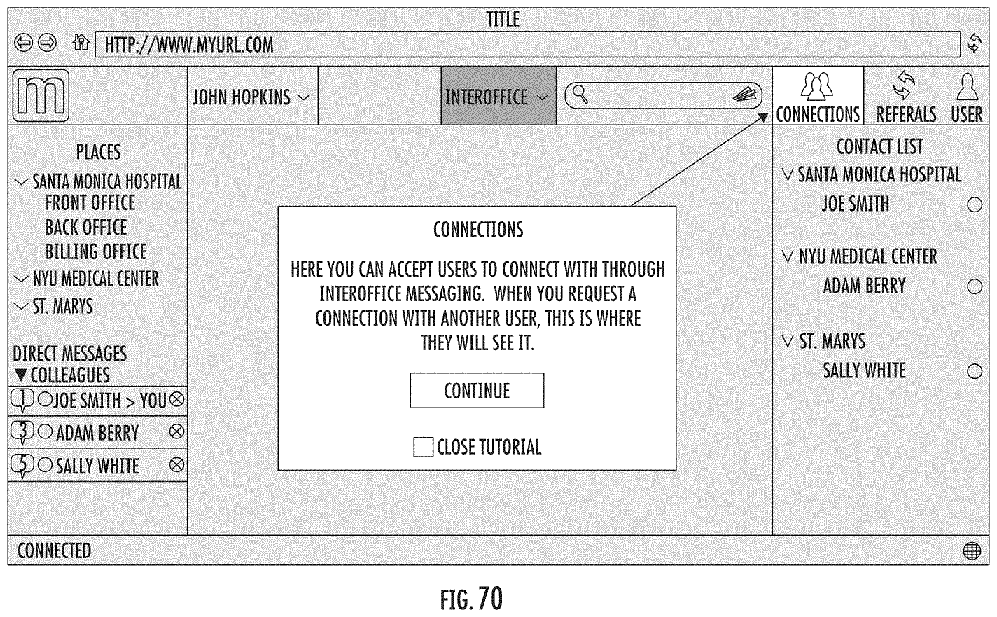

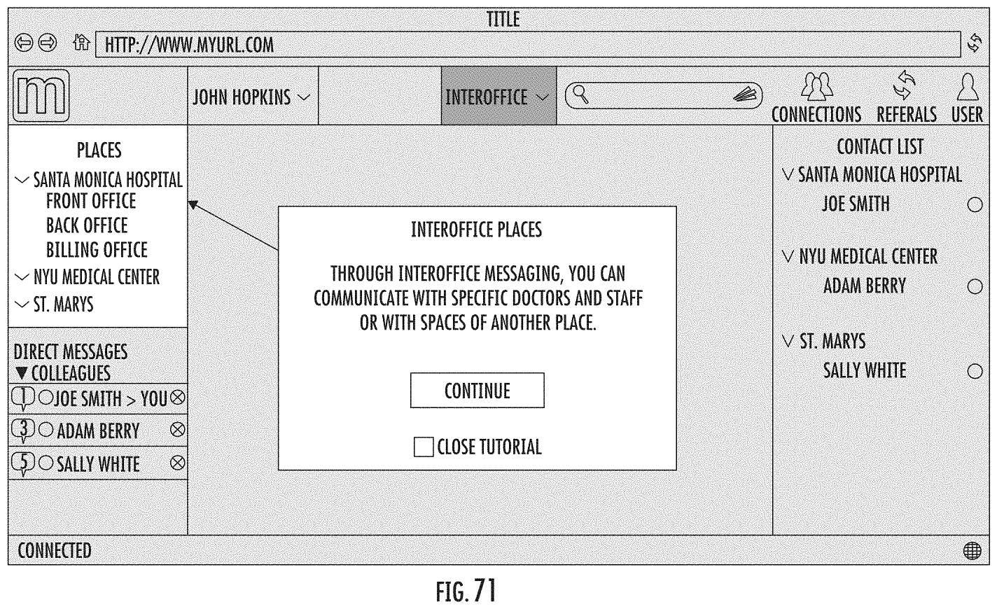

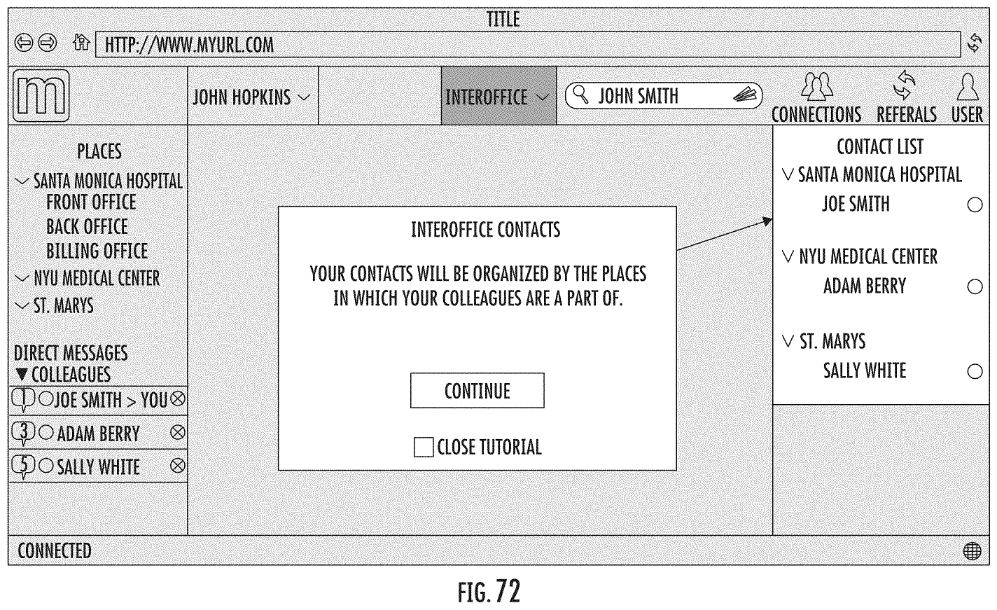

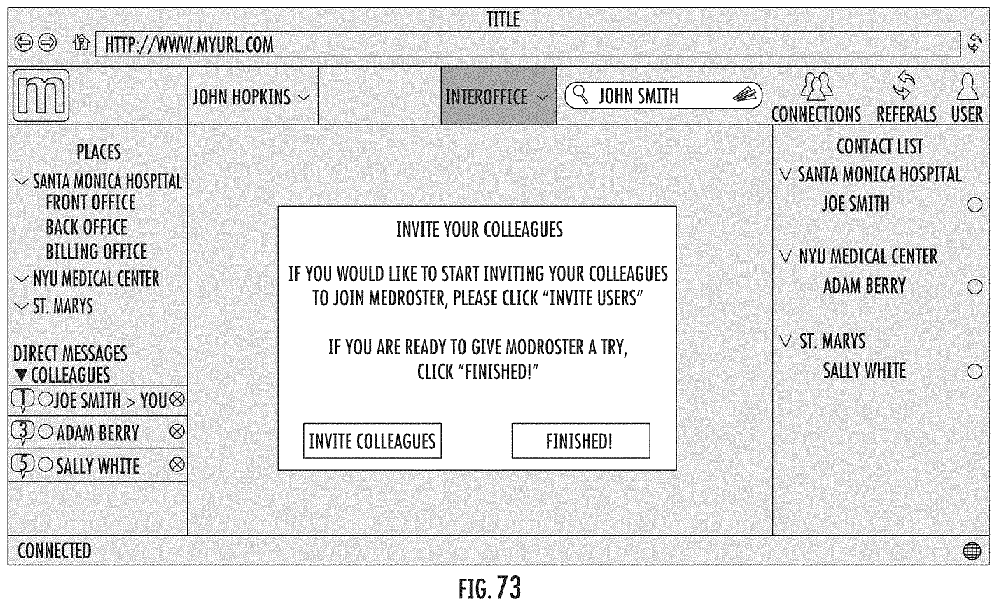

FIGS. 62-73 shows screens for an onboarding tutorial. For example, after signing up for the encrypted messaging system, a doctor/staff user may be directed to their dashboard. When they are registered, the encrypted messaging system may also give the user a walkthrough of the site in order to familiarize them to the environment. Also, at the end of the landing tutorial, the user may be given the opportunity to invite their colleagues to try the encrypted messaging system. For every screen, if the user clicks “continue”, the screen may move to the next one in the sequence. If they click the “close tutorial” checkbox, they may be directed to their dashboard.

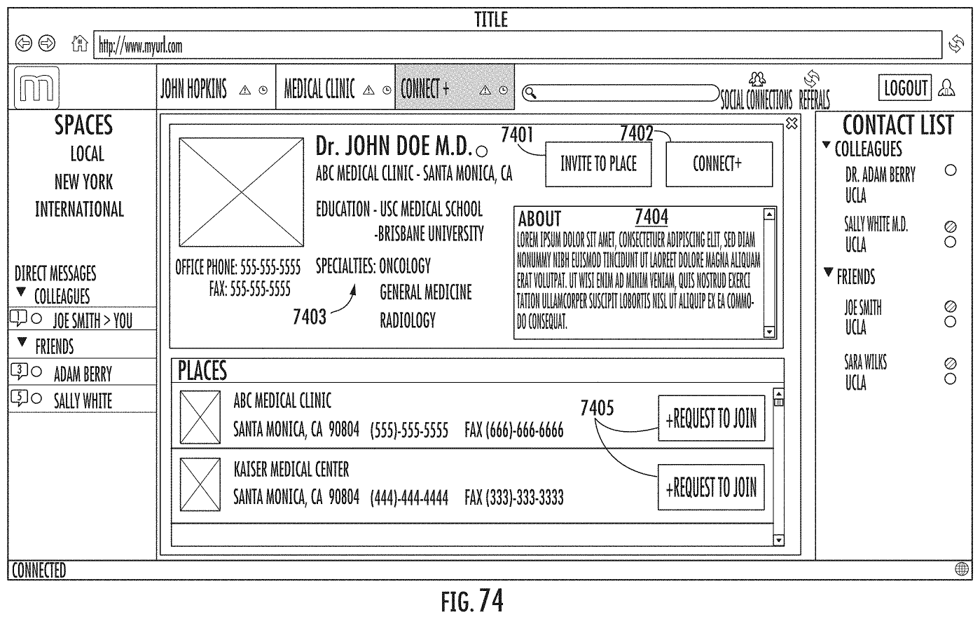

FIG. 74 shows a screen for a user profile. This allows users/places a profile to provide other users easy access to information, ways to connect and gives the encrypted messaging system a more social feel. The profile is broken down into two sections: user information and place information. FIG. 74 includes:

7401. Pressing “invite to place” may send the user an invite to join a user's place (see FIG. 46)

7402. Pressing the “social connection” button may send the user a “colleague request”, and if accepted, may add the user to the users in your social place (see social connection spec)

7403. This is where a user's main information may be, including education and specialties.

7404. When a user edits their profile and creates an “about me” section, this is where that information may be viewed.

7405. This section may list off the user's places that he is a member of. Clicking “request to join” may send that place's admin a request to be a member of the place.

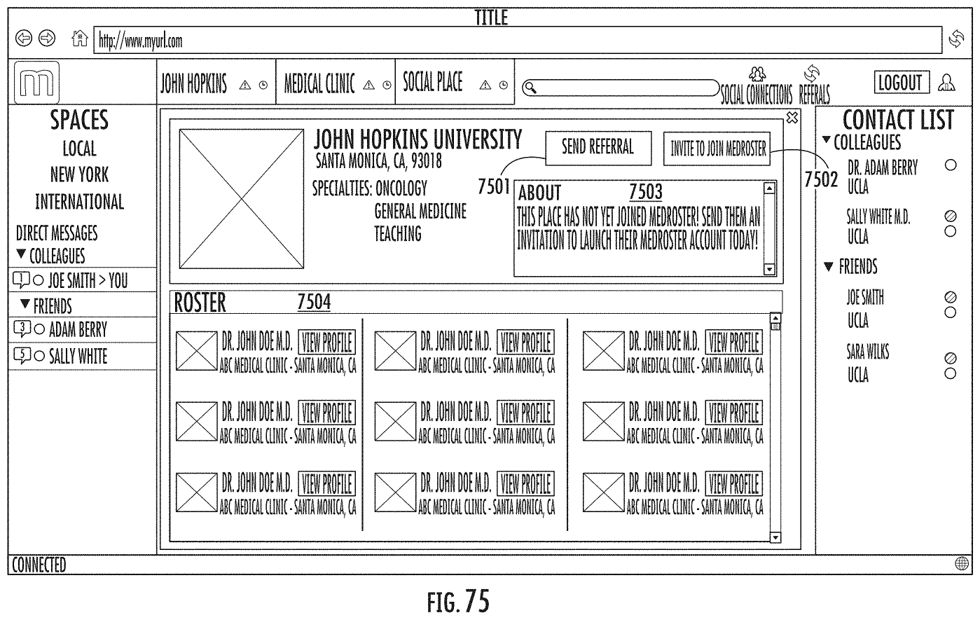

FIG. 75 shows a screen for a place profile. The place profile is similar to the user profile in that it gives more detailed information about the place and different ways a user can connect with that place. FIG. 75 includes:

7501. Selecting “send referral” may open a modal that may give the user the ability to send a referral directly to a place through an autofill list (see send place referral diag)

7502. “invite to join Medroster” may on be visible if the place has not been activated through the encrypted messaging system yet. If they are active, the button may read “request to join place.” Clicking “request to join place” may send that place a connection request that may allow that user to join the place.

7503. This may show information about the place.

7504. This “roster” may show which users are connected to/members of this place. It may also give the user to option to view the profiles of those users and connect with them that way.

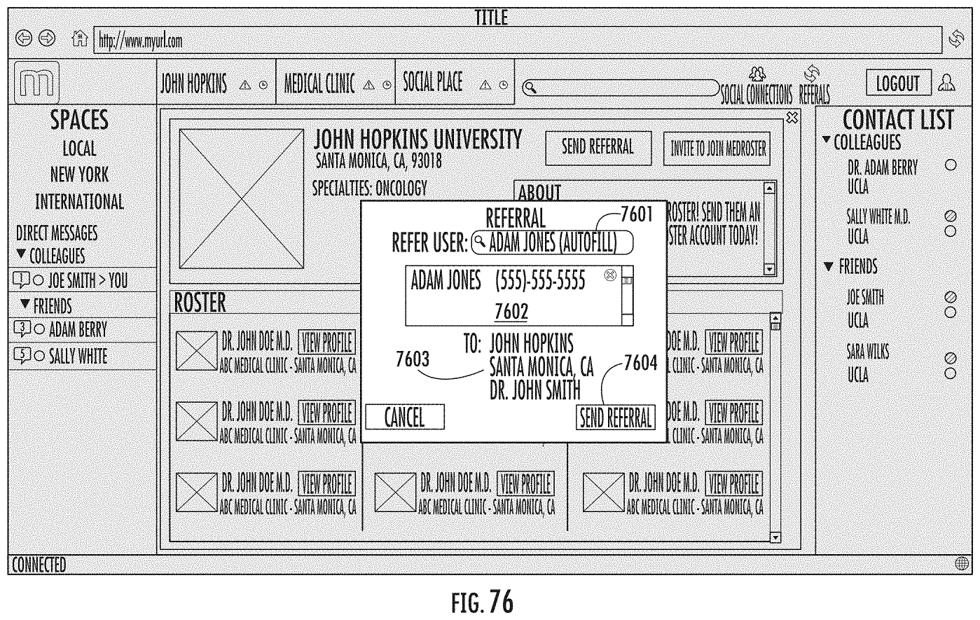

FIG. 76 shows a screen for a place profile referral. This modal may provide the user a quick way of sending a patient referral to another place. FIG. 76 includes:

7601. The doctor/staff may type the doctor's name which they wish to refer into an autofill list.

7602. After clicking return, the patient's name/info may fill this screen. Multiple patients may be added to this list and referred at one time.

7603. This shows where to patient is being referred to.

7604. After clicking “send referral”, the referred patient's info may be sent to the place that was selected. The patient's info may also be added to the “sent referrals” of the user's current place.

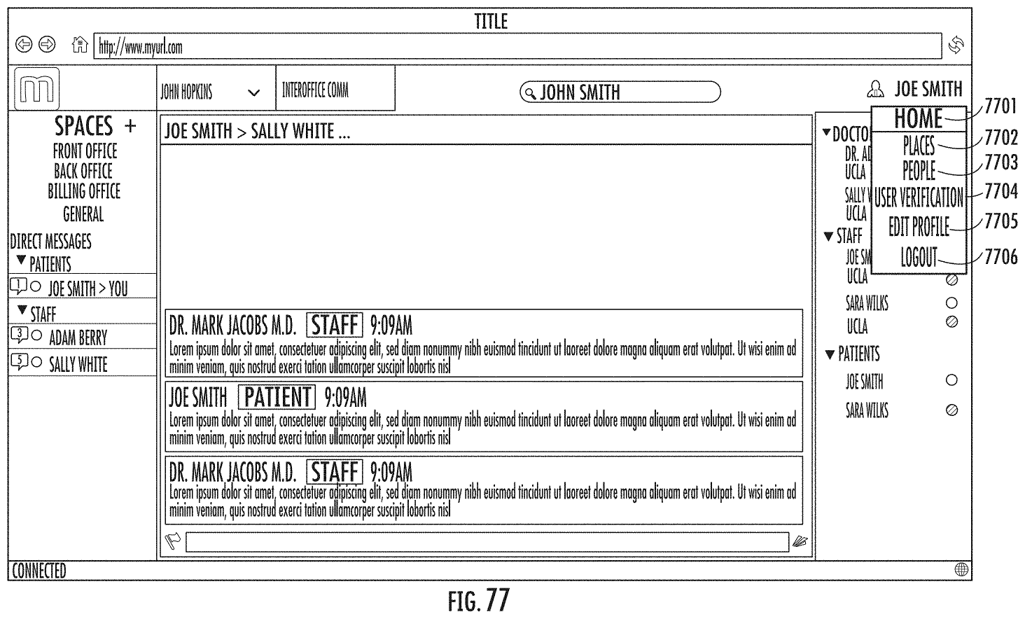

FIG. 77-83 shows screens for a user dropdown specification. The user dropdown may be accessed on the right of the top navigation bar and organized to follow a hierarchy. Access to certain features of the user dropdown may be limited depending on a user's type. For example, only administrators can edit people, places, place profiles or people profiles, and user access control.

FIG. 77 shows a screen for a web application dropdown. FIG. 77 includes:

7701. Home: Goes to Dashboard of last visited place

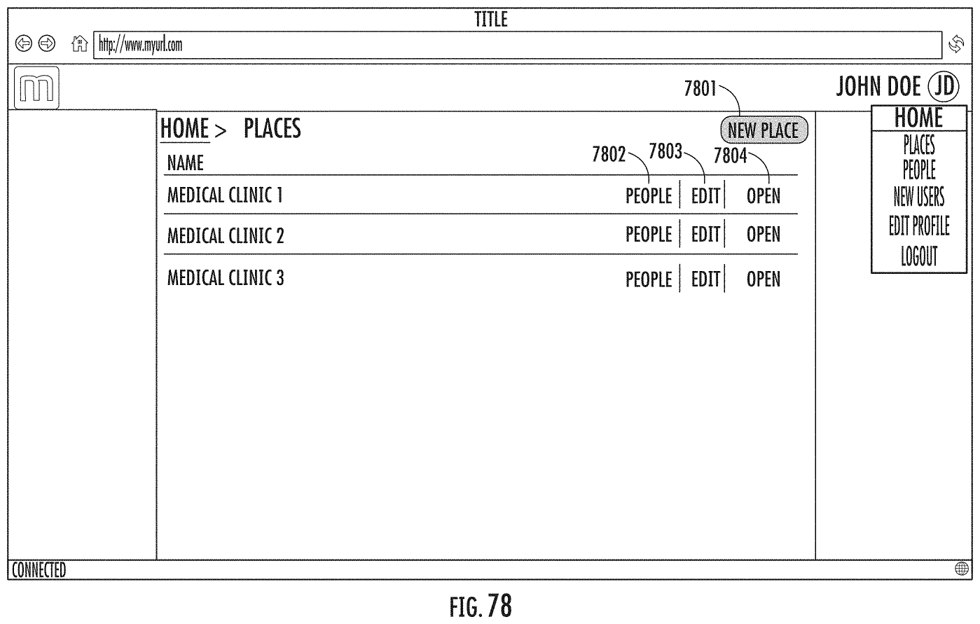

7702. Places: to screen shown in FIG. 78

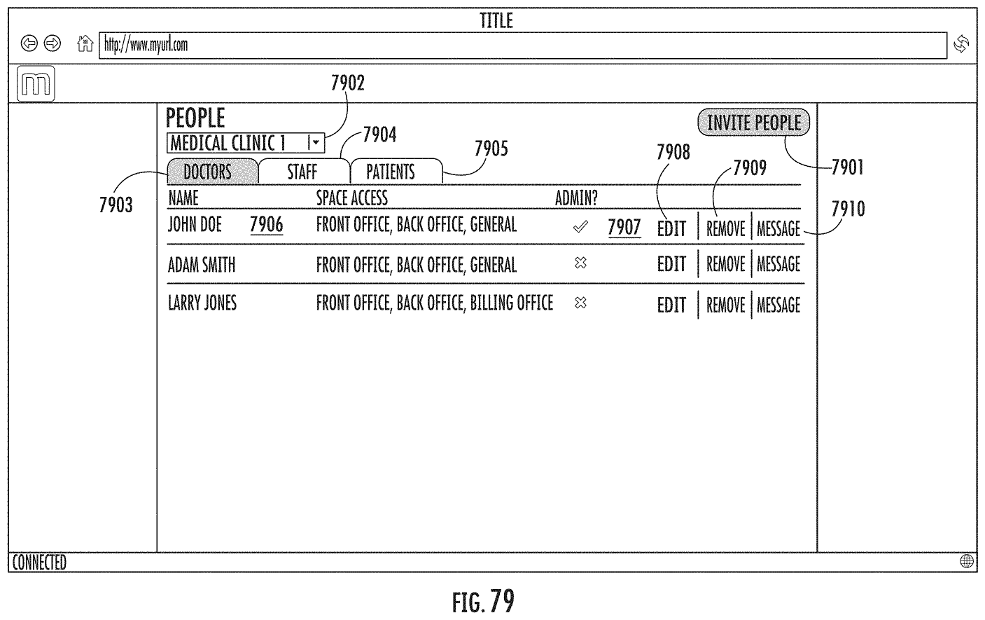

7703. People: to screen shown in FIG. 79

7704. User Verification: (discussed elsewhere)

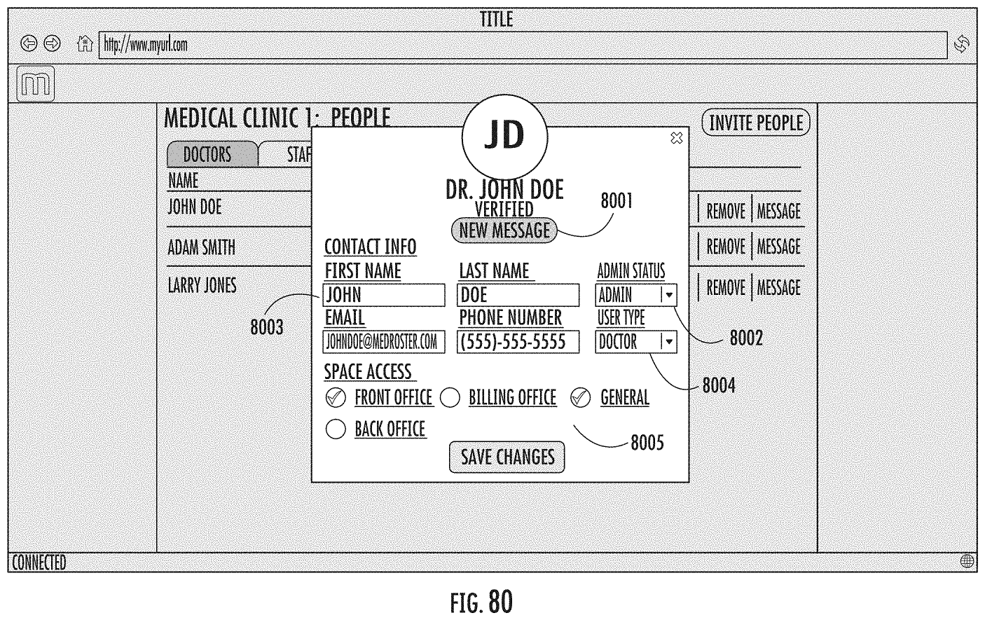

7705. Edit Personal Profile: to screen shown in FIG. 80

7706. Logout: Signs user out of the encrypted messaging system and opens homepage.

FIG. 78 shows a screen for web application place page. FIG. 78 includes:

7801. “New Place”: Brings user to registration process. Because the user has already entered all of their personal information into the system, they may skip that part and go directly to the places search screen. This may be further explained in the “New Place” spec.

7802. “People”: to screen shown in FIG. 79

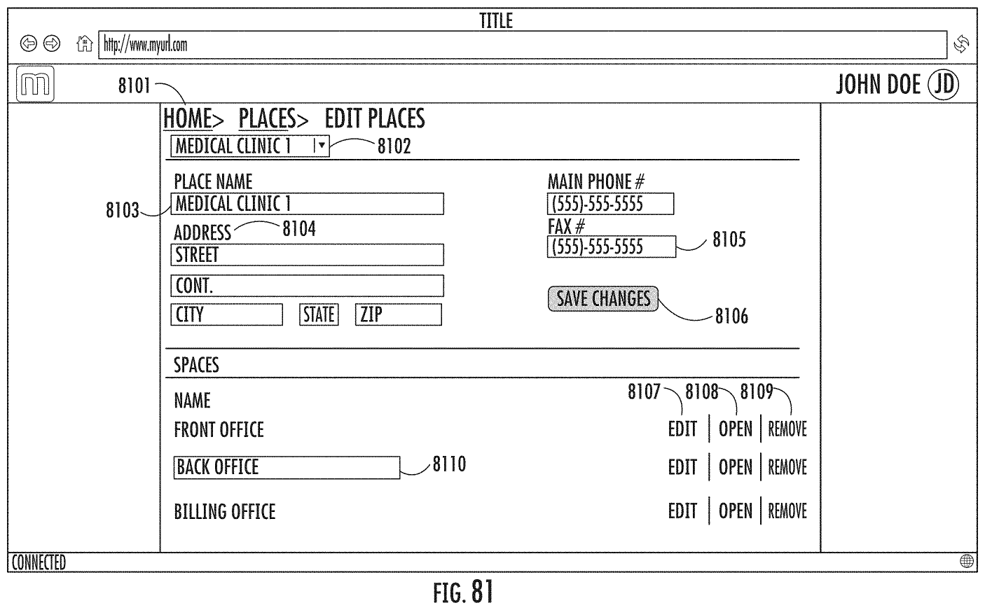

7803. “Edit Places”: to screen shown in FIG. 81

7804. “Open”: Opens dashboard of selected place.

FIG. 79 shows a screen for web application people page. FIG. 79 includes:

7901. “Invite People” button opens invitation modal

7902. This dropdown allows the user to switch between places. When a user selects a new place, the screen may refresh to show that user database.

7903. The “Doctors” tab may show only doctor users in the specific place chosen.

7904. The “Staff” tab may show only staff users in the specific place chosen.

7905. The “Patients” tab may show only patient users in the specific place chosen.

7906. Each row may show a different user's name, spaces they have access to, and whether the user is an administrator or not.

7907. If the user listed has administrator status, a “check” may fall under the admin category. If not, there may be an X.

7908. “Edit” link opens to FIG. 80, a modal to edit a user's personal info.

7909. “Remove” link opens a dialogue box to remove the user from the place.

7910. “Message” link may open a conversation with the user in the dashboard.

FIG. 80 shows a screen for editing a user profile. FIG. 80 includes:

8001. “new message” button may open a new conversation with the user in the dashboard.

8002. “Admin Status” dropdown may contain the selections “Admin” and “Non-Admin.” If “Admin” is selected, that user may have all administrator capabilities.

8003. User Info

8004. “User Type” dropdown may contain the selections “Doctor” “Staff” and “Patient.” This may affect the user's labeling throughout the site.

8005. Checking a space group may allow the user access to that specific space. Unchecking a space group may disallow the user from access to the space.

A “Save Changes” button may update all changed information in the database.

FIG. 81 shows a screen for editing a place. FIG. 81 includes:

8101. Home-goes to dashboard of selected place

8102. Dropdown list of user's places

8103. User-facing name of Place. May affect database place name and name site wide

8104 and 8105. for place profile/database

8106. May update place information/database site wide

8107. “Edit” may let the user change the name of a space (see point #10.)

8108. “Open” may take user to dashboard with selected space open

8109. “Remove” may open a modal asking if the user is sure they want to remove it (Remove/cancel options)

8110. View of “Edit” space.

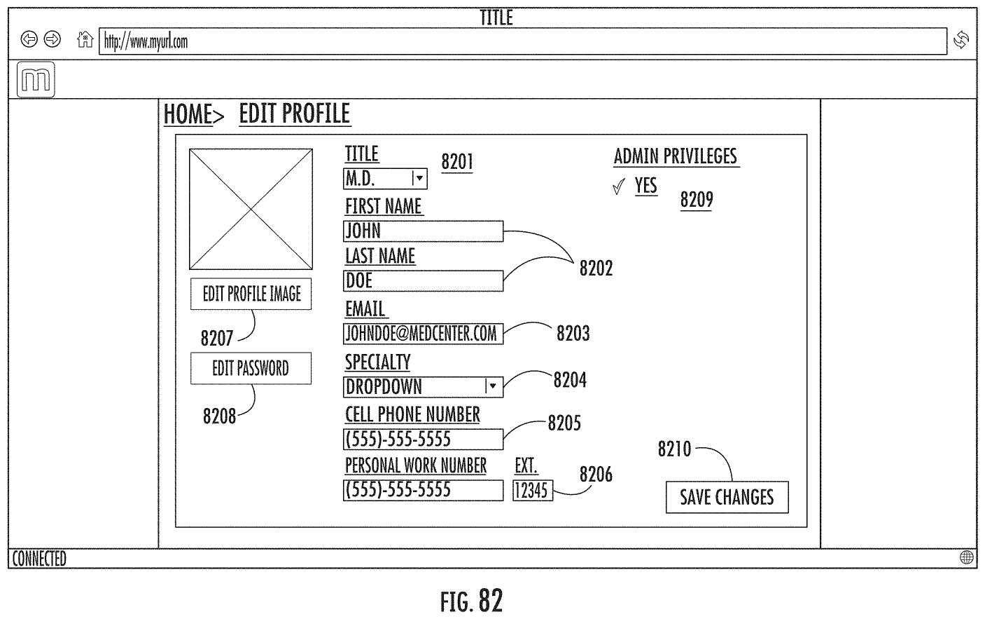

FIG. 82 shows a screen for editing a personal profile. FIG. 82 includes:

8201. Title: Use http://www.pamf.org/physicians/titles.html for list.

8202. User Info

8203. email

8204. Specialty: Use data available at http://www.mclarenhealthplan.org/HealthAdvantage/AListofDefinitionsofMedicalSpecialties Adv.aspx for list.

8205. Cell phone number—used to cross reference with Dr App

8206. Work number: Number that is called when user is in patient's contact list and patient presses the “phone” button.

8207. Edit Image: Image applies as profile image for user site wide. Opens file browser to choose image.

8208. Edit Password

8209. Admin Privilege: Shows Check if user is admin, X if user is not

8210. Saves all changes and takes user to previous page.

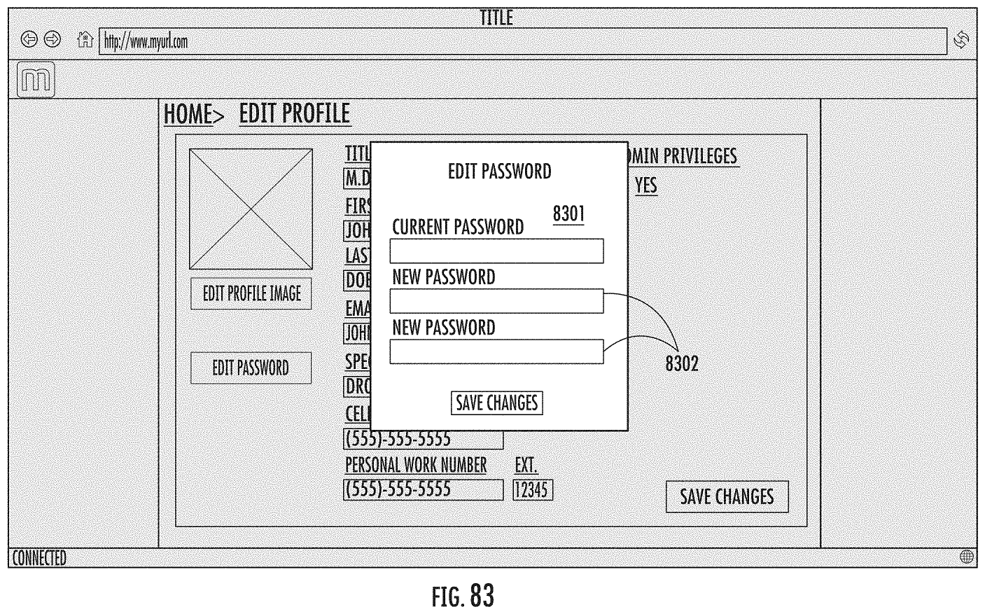

FIG. 83 shows a screen for editing a password. FIG. 83 includes:

8301. current password must match the user's current password

8302. New passwords must match.

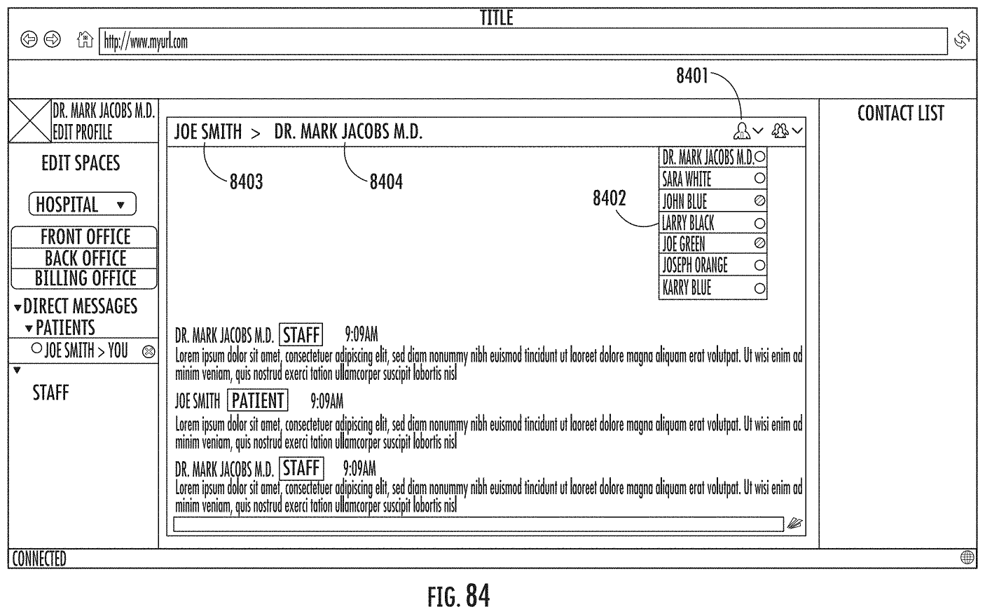

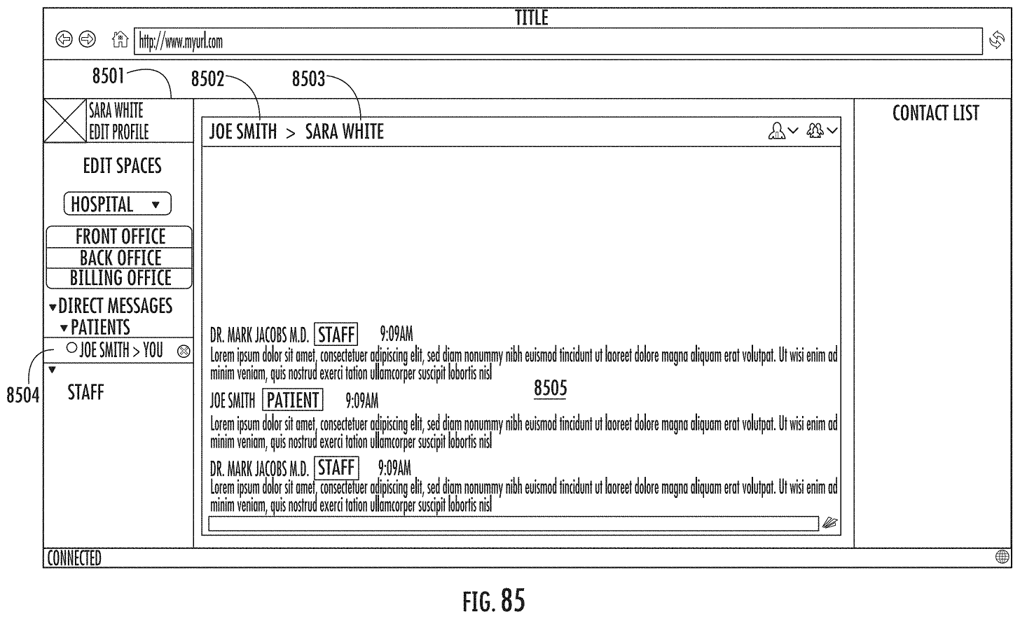

FIGS. 84-85 show screens for a transferred user interface. For example, the transferred user interface may be used to allow a transferring user, such as a Doctor/staff user (A) passing off a conversation with a Patient (P) user to another doctor/staff user (B). When this happens, the A-P conversation is closed, the whole A-P conversation is copied over to the B-P conversation. The encrypted messaging system may check whether a conversation was already passed previously, so that those messages may not be copied again (only copy messages from the last transfer point onward). B and P may be able to view the correspondence between A and P, in “their” conversation, and continue the discussion themselves. FIG. 84 includes:

8401. This Icon, when clicked, may provide the user of a dropdown list of users (excluding patients) that are allowed access to this specific space.

8402. Selecting one of these users may “transfer” the entire correspondence of the visible chat to whomever was selected. Doing this may effectively remove the correspondence from your control to the selected user.

8403. Patient/User that the transferring-user is speaking with—to be transferred

8404. The transferring user (yourself).

FIG. 85 shows a screen that depicts the user seeing the correspondence they received after said transfer. FIG. 85 includes:

8501. receiving user

8502. transferred user (with correspondence)

8503. receiving user

8504. the correspondence may appear under your direct messages as a new correspondence

8505. all of the correspondence between the transferring user and the transferred user may be available for viewing, and from this point the transferring user is no longer in control of this correspondence. This is now an active correspondence and the receiving user may continue to chat with the transferred user if necessary.

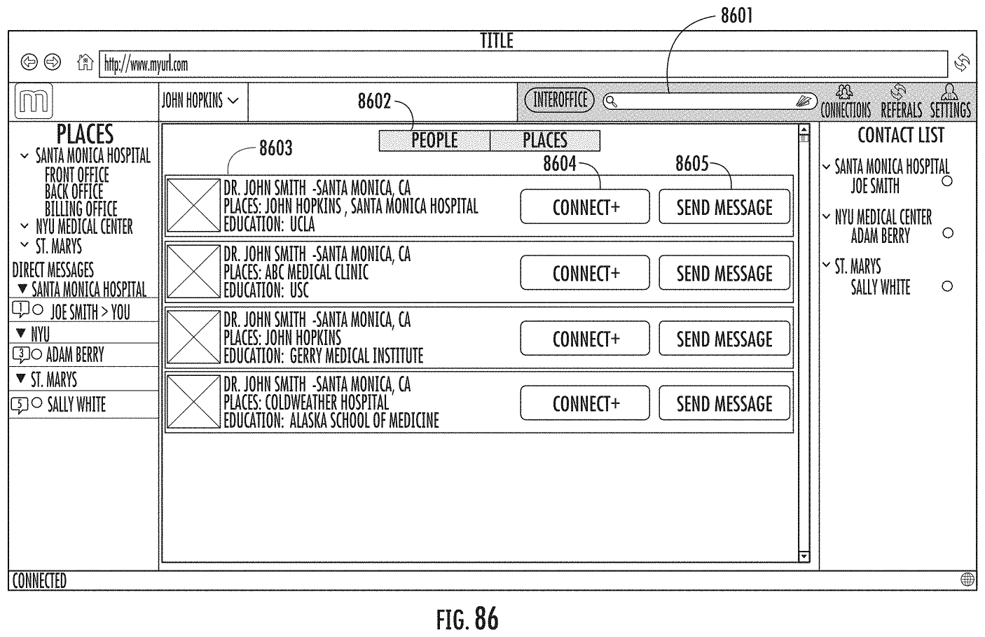

FIGS. 86-89 show screens for a people/places search feature. To find colleagues to invite to join places, create a connection or send referrals, doctors/staff may need the ability to search the database. Doctors and staff may be able to search for other doctors or other places, view user/place profiles and send messages/connection requests. FIG. 86 shows a screen for a colleague search. FIG. 86 includes:

8601. To initiate search, a doctor/staff can start typing in the search bar on the top nay. Once the doctor/staff hits return, the search results screen may show all users that fit the searched text.

8602. Doctors/staff may have the option to search for doctors and places. Selecting “people” may search doctors in the database. Selecting “Places” may search places by name or address

8603. Once a name is entered into the search field, doctors with matching names may fill the page. Along with the doctor's name, their location, place and education may show to provide the searching doctor/staff with a more information to help them find the correct user.

8604. Clicking “connect+” may and the selected user to the original users contact list.

8605. Clicking “send message,” may give doctors/staff a quick way to message another doctor.

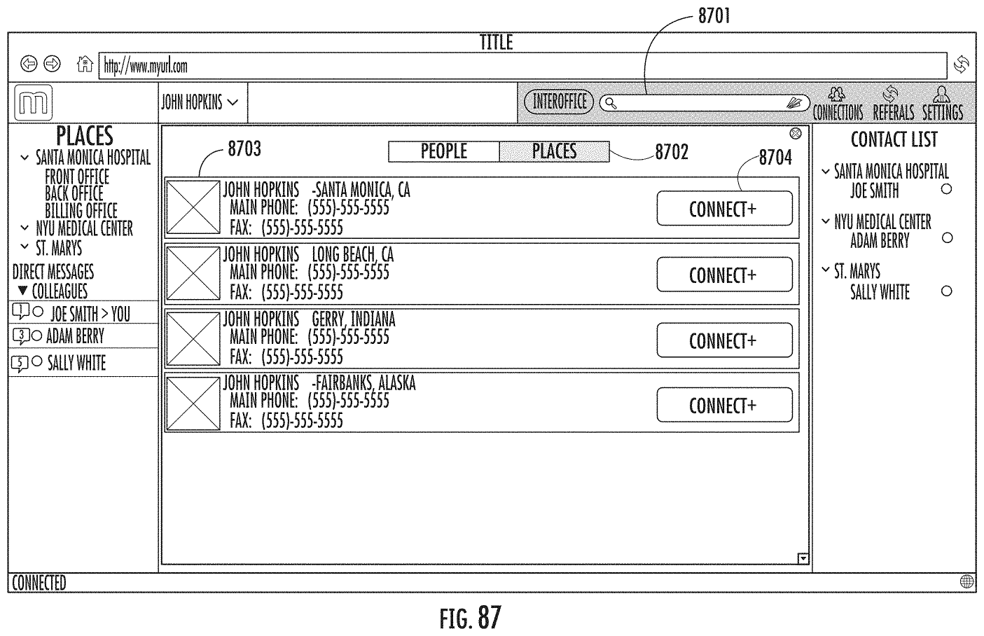

FIG. 87 shows a screen for a place search feature. FIG. 87 includes:

8701. To initiate search, a doctor/staff can start typing in the search bar on the top nay. Once the doctor/staff hits return, the search results screen may show all places that fit the searched text.

8702. Doctors/staff may have the option to search users and places. Selecting “people” may search users. Selecting “Places” may search places by name or address

8703. Once a place is entered into the search field, places with matching names may fill the page. Along with the place name, the searching doctor/staff may see thee place's main phone line and fax phone number.

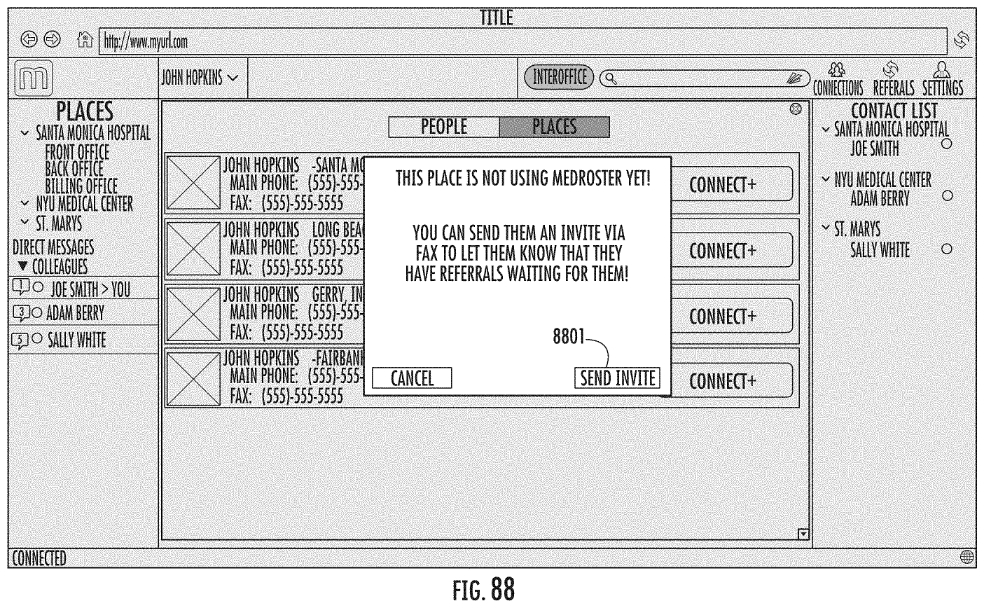

8704. Clicking “connect+” may add the place to the users “Places” list on the left sidebar. Under the place, the spaces of that place may be listed. The user may now be able to communicate with the spaces of that place in the same way a patient would be able to communicate with the spaces of that place that he/she belongs to. If the user attempts to connect with a place that is not currently active on the encrypted messaging system, the user may be directed to the screen shown on FIG. 88. There, the user may be prompted that the business is not active and given the option to invite the place by Fax.

FIG. 88 shows a screen where a place is not found. In the case a place is searched and has yet to become active on the encrypted messaging system, the searching doctor/staff may view this message. They may have the option to send an invite to that place. FIG. 88 includes:

8801. If the doctor/staff chooses to invite the place to join the encrypted messaging system, that place may receive a FAX to the fax number we have in the database for that place. The fax may give information on who is inviting them, how to join, and if they have referrals waiting on the encrypted messaging system.

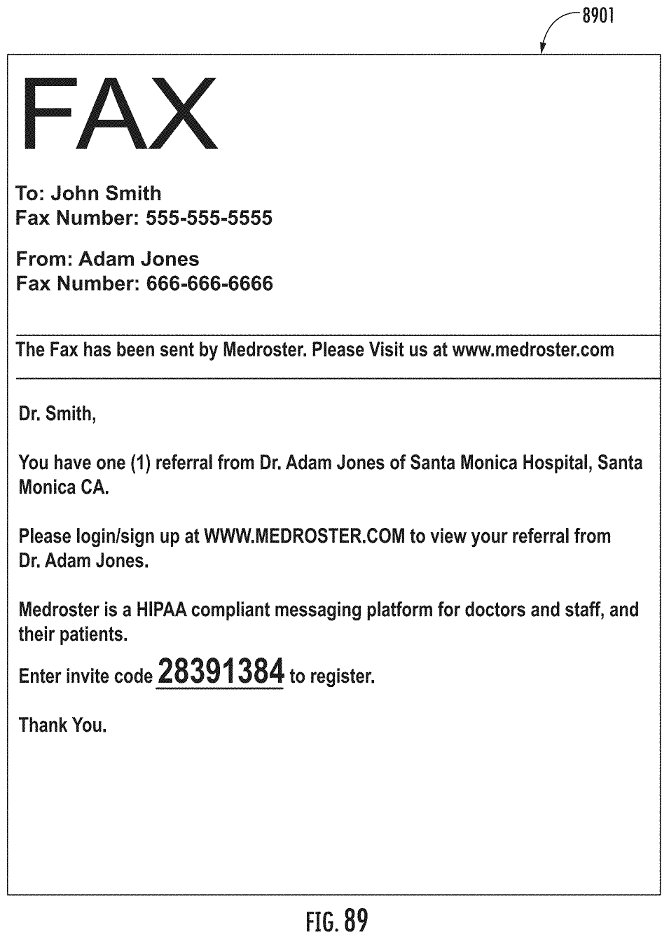

FIG. 89 shows a fax a user may use to sign up for the encrypted messaging system, as shown in 8901.

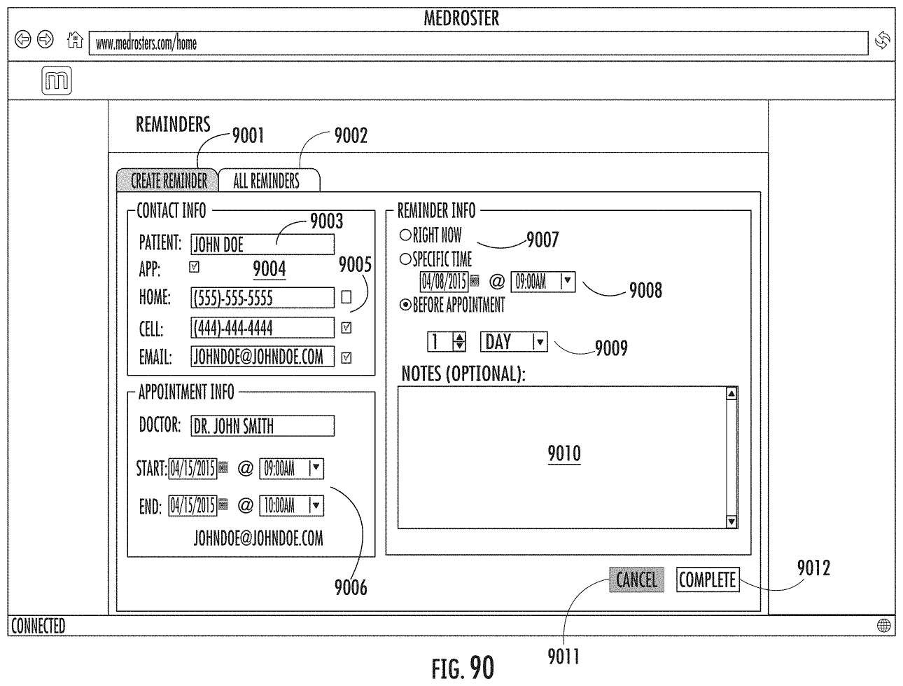

FIGS. 90-93 show information of a reminders feature in a web and messenger application. The Reminder Feature functions as a way for Staff users to communicate with patient users by Phone, text, email, or through the application in the present or at a predetermined time in the future. FIG. 90 shows a screen for a staff to create reminders. FIG. 90 includes:

9001. Create Reminder Tab displays this interface

9002. All Reminders interface tab

9003. “Contact info”: Patient Name text field is autofill, and may fill in other fields (Home #, cell #, email address)

9004. Checkboxes: the patient user may receive the “reminder” at all checked selections (e.g., this user may be reminded through the application, they may receive a text to their cell phone, may receive an email.)

9005. Cell # may always receive text message

9006. Appointment times: The patient user may receive the following information when a reminder is sent to them: Place where appointment may be held, doctors name, Date/time of appointment, and notes (if provided).

9007. Right Now: this may immediately send the patient user the message when “complete” is pressed.

9008. “Specific Time”: This may send the patient user a reminder at the exact time selected.

9009. “Before Appointment”: Selecting this option may use the patient's appointment time as the variable. The options for the spinner should go no higher than 7, and the options for the drop down should be: Day, Week, Month.

9010. “Notes”: Any extra information the staff should wish to provide may go in this text field (optional). All other information is necessary to complete the form.

9011. “Cancel”: Form may delete, and bring the user back to the dashboard

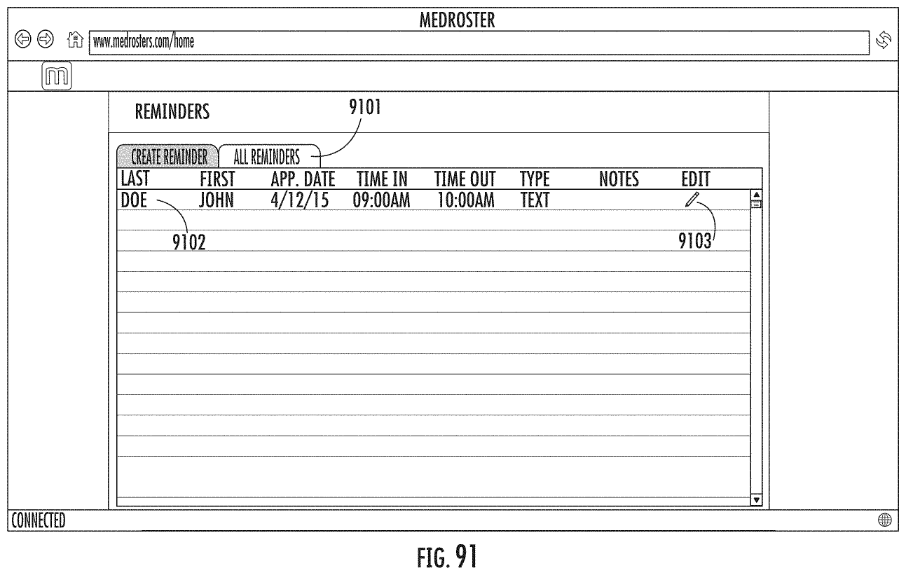

9012. “Complete”: Selecting “Complete” may archive the reminder until it is meant to be sent or send immediately if the staff user has selected that option. It may also send the reminder to the form on screen shown in FIG. 91 (All Reminders).

FIG. 91 show a screen for all reminders interface for staff. FIG. 91 includes:

9101. Selecting the “All Reminders” tab may display this screen

9102. After a reminder has been completed, all data from said reminder may display here

9103. Selecting the “edit” button may display the “Create Reminder” screen, but all information from the selected reminder may be completely filled in. Selecting “Cancel” may delete the reminder from the list, and it may not be sent to the patient user.

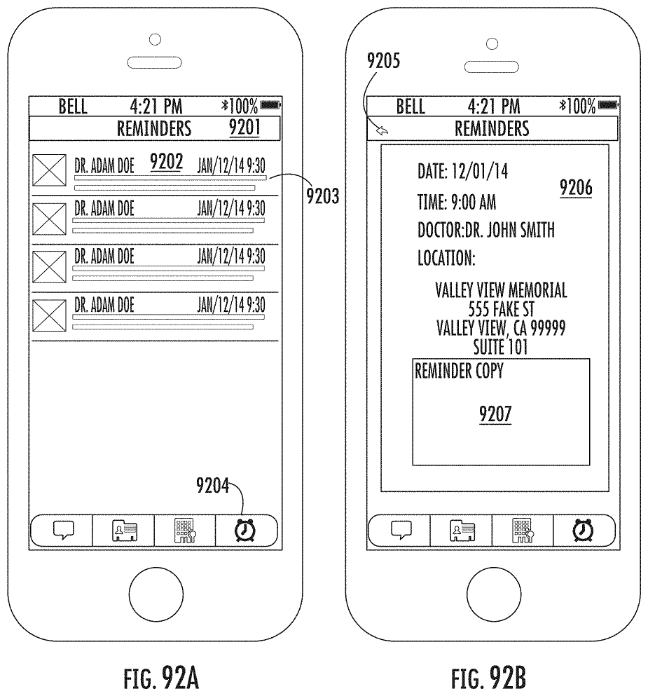

FIGS. 92A-92B show screens for a reminders interface for patients. FIGS. 92A-92B includes:

9201. Selecting the “Reminders” Icon (4) may display this screen

9202. “Reminder Summary List” may display the doctors name, the date/time of the patient user's appointment (NOT TIME RECEIVED) and a short sample of text from the “notes” field followed by ellipses similar to a message summary in history

9203. Date/Time of appointment

9204. “Reminders” Icon

9205. “Back Button” may bring the user back to the reminders screen

9206. “Reminder”: All information from this screen is pulled from the fields of FIG. 90 except the location. The place information may be loaded automatically depending on where the reminder was sent from

9207. Any text from the Notes field may appear here

Reminders may disappear after the appointment date has passed.

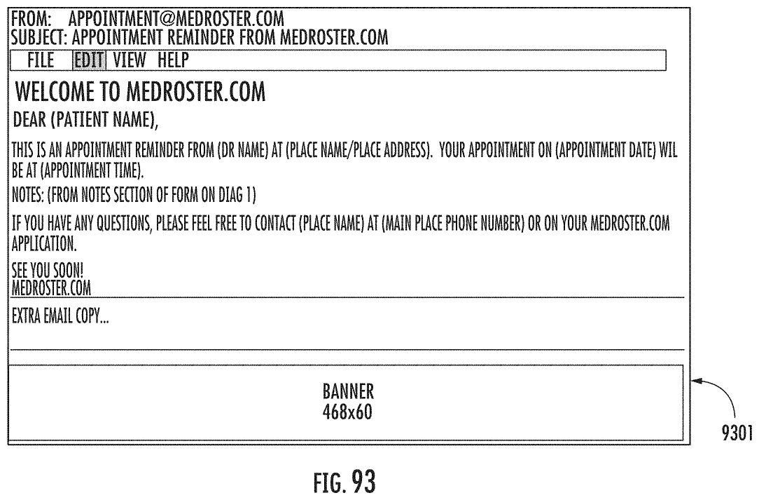

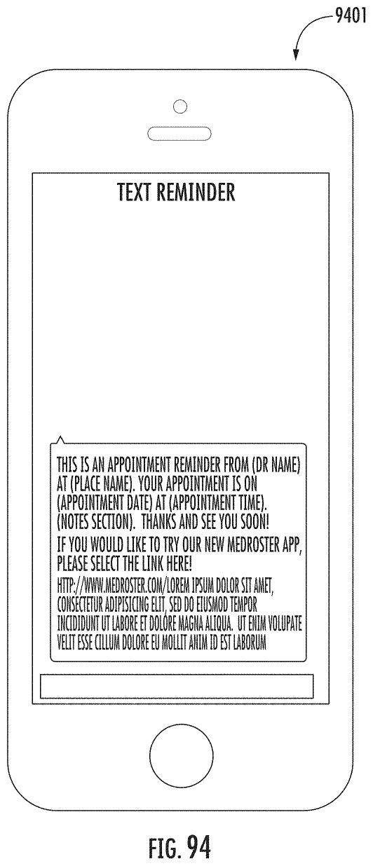

FIG. 93 shows a copy of an email for a patient reminder, as shown in 9301. User variables from patient reminder form (e.g., FIG. 90) may be inserted in relevant portions of the email. FIG. 94 shows a copy of an in-application patient reminder, as shown in 9401.

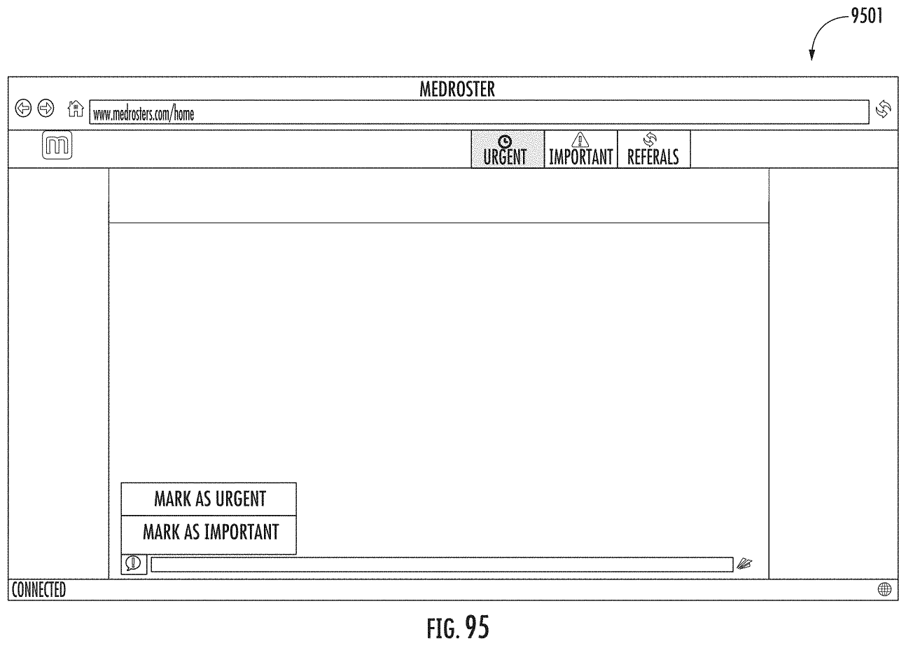

FIGS. 95-98B show screens for a chat feature in a web messenger application. FIG. 95 shows a screen for the chat feature in the messenger. When a user selects the (!) icon to the left of the text bar, the urgent/important box may pop up. When selected, the message may be flagged as either urgent or important.

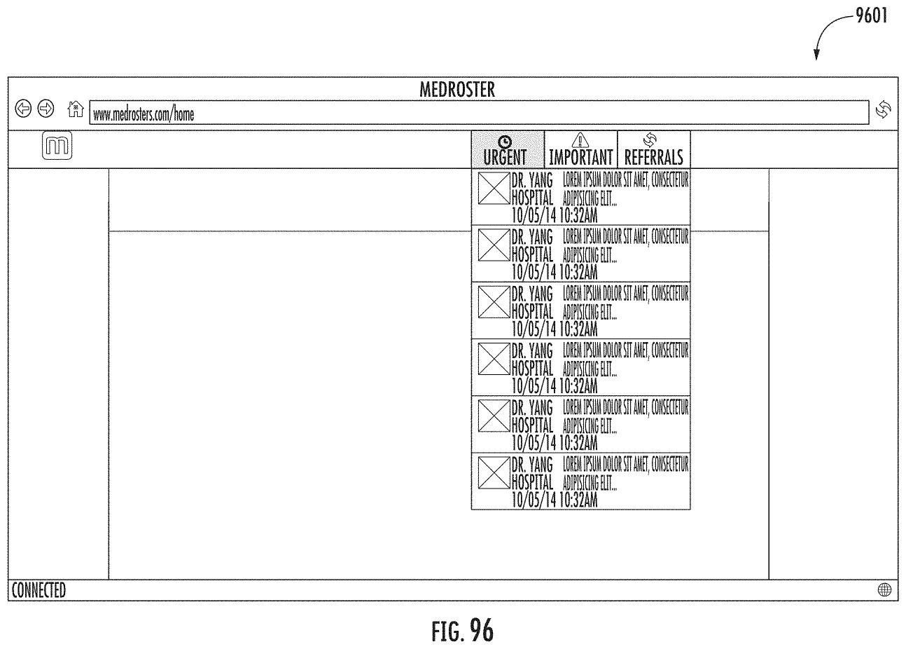

FIG. 96 shows a screen for notification, as shown in 9601. When a user receives a message that was marked as either urgent, important, or is a referral, they may be notified here. Each may be a drop down. Each entry is an unread/important message. When an entry is selected from the dropdown, the conversation may open up on the user's dashboard and the selected message may be scrolled into view. Each entry has a button that allows closing it (and removing it from the Urgent/Important menu). The menu may include a small number icon to show how many urgent messages remain.

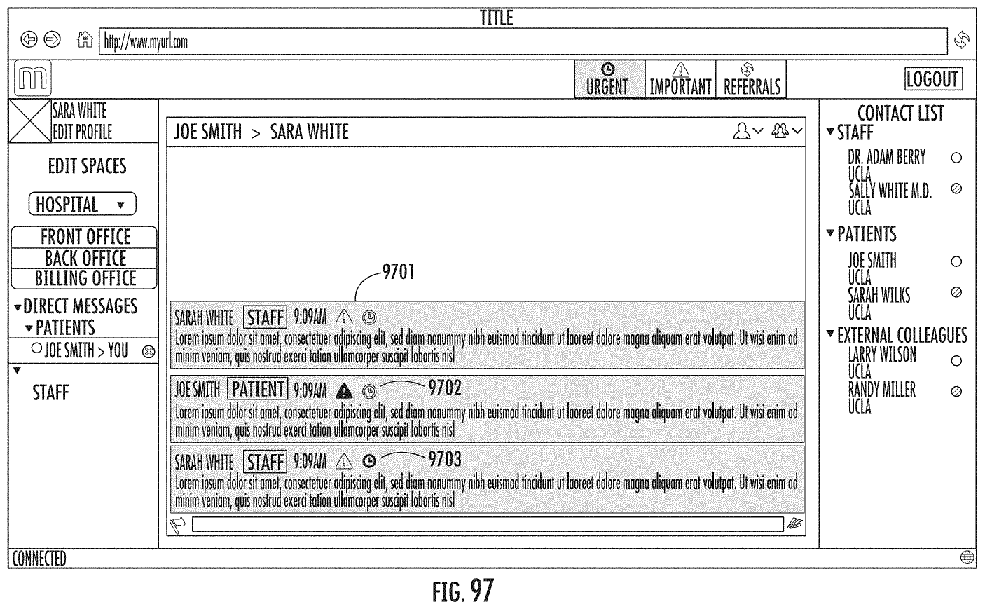

FIG. 97 shows priorities features. The way a user selects an urgent/important message is still the same. The only thing that has changes is the way the user can mark/unmark the message after it has been sent. FIG. 97 includes:

9701. This shows the state which shows the message is neither important nor urgent. These icons are clickable. If the icon is not marked, the user now may gain the ability to mark any message urgent/important after the fact

9702. This shows the icon “important” marked. This may look the same whether the sending user sends the message marked important, or if the receiving user deems the message to be important

9703. This shows the message marked urgent. It has the same functionality as the important icon. Both urgent and important can be marked at the same time.

If the user wants to mark a message they have received as urgent/important, they may be able to do so, but it may not be visible to anyone else seeing that message. This may happen when a user marks a message before they send it to that user.

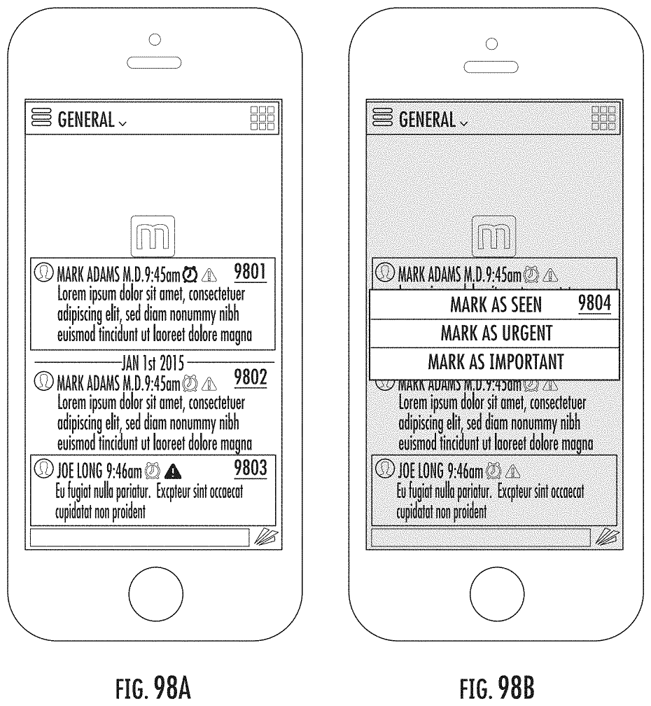

FIGS. 98A-98B show screens for the chat feature in a mobile application. Urgent/important messages should have a color-coded indicator on the history list and in the conversation view. Red exclamation for urgent. Yellow or orange symbol for important like a warning sign. FIGS. 98A-98B includes:

9801. Urgent message

9802. Important Message

9803. Holding message may dim screen and display message

9804. Pressing “Mark as Seen” may show the message as normal. No color or icon. May also remove from notifications.

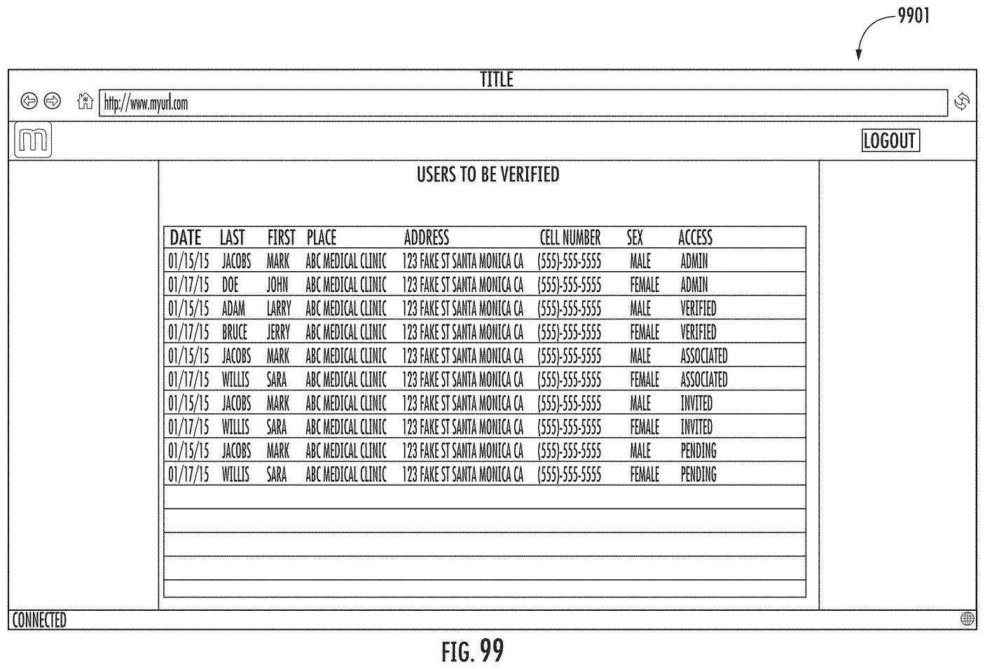

FIG. 99 shows a screen for a user waiting to be verified, as shown in 9901. Access to this feature may be limited to doctor and staff type users, not for patient type users. While a user (doctor or staff) is on “hold” because their account has yet to be verified by their place admin, they may view this screen. It may show the user their info and any other user info that are also waiting to be verified by the same place. Once the user has been verified, they may be given access to the dashboard and may no longer see this screen. All users placed in ONE office location and status listed and action can be executed: (1) user . . . active, pending activation, verified, pending verification, HIPPA authorized and (2) invite (by email, txt, NPI, office #), verify (by fax, phone), and authorize by owner(admin or doctor). FIG. 99 includes User info from sign up process; “Admin” is admin user of place; 3. “Verified” is a verified user of the place; “Associated” is a user that is verified by association to another doctor; “Invited” means they are invited but have not been verified yet; “Pending” is a user that signed up on their own but have not been verified yet.

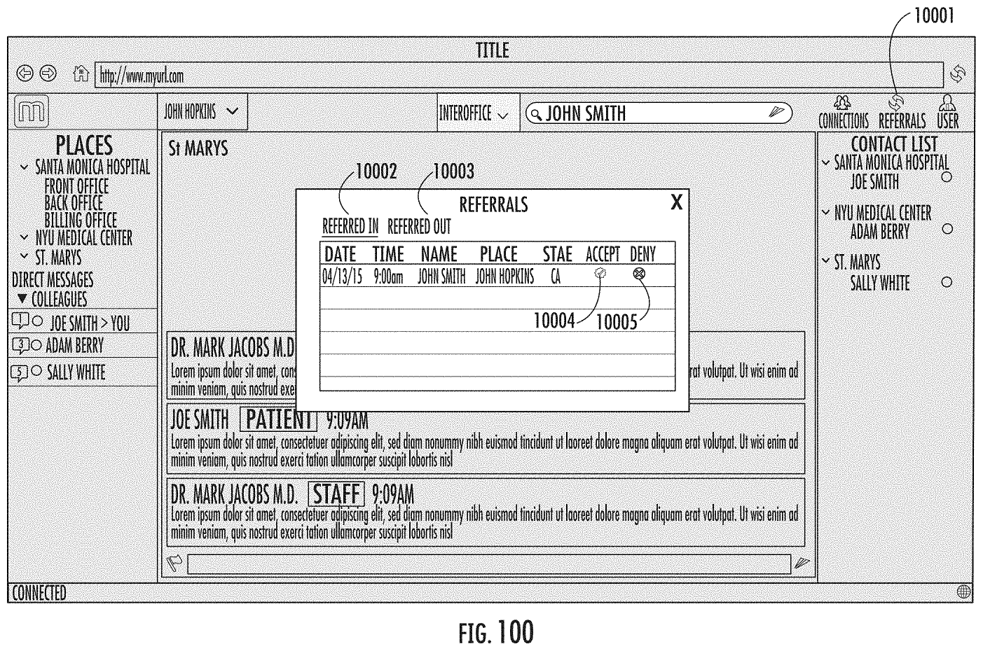

FIG. 100 shows a screen for a referral modal. FIG. 100 includes:

10001. Press to open modal

10002. “referred in” are referrals received from other doctors/places

10003. “referred out” are referrals sent from your place

10004. “accept” button may add the user to your “patients” contact list

10005. “deny” may remove the user from this list.

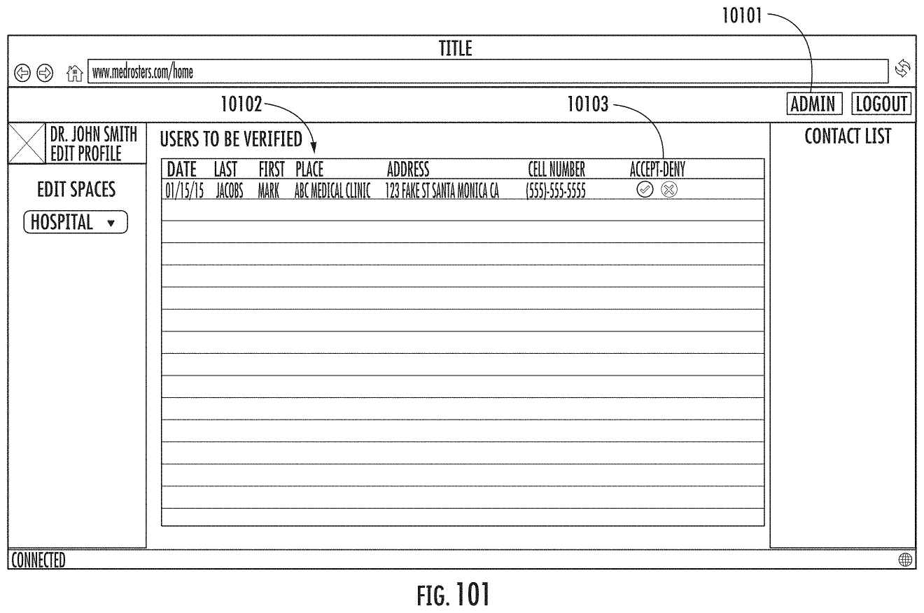

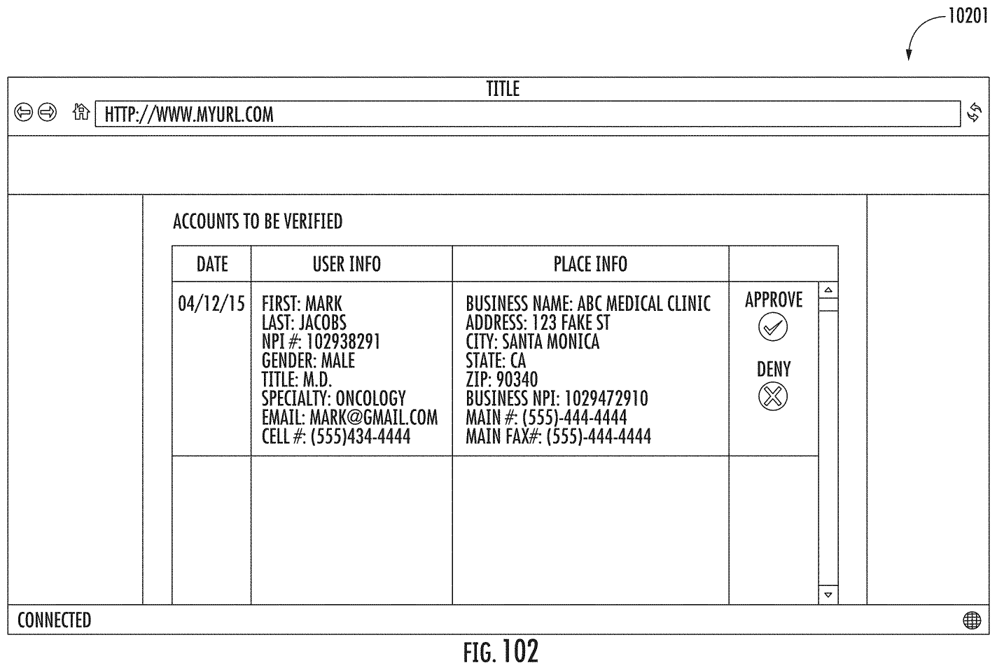

FIGS. 101-102 show screens for an administrator to manually verify a new user. After a user completes their sign up process to join an already existing/operational place in the encrypted messaging system, the ADMIN of that place may view this form that may give detailed information about the user attempting to join, and be given the option to accept/deny access to their place. FIG. 101 includes:

10101. Admin access from this button

10102. All information visible may be pulled from the forms of the user's registration process. Place/address may be the place that the user is attempting to join

10103. Pressing the accept button may add the user to the place. They may have full access (besides admin privileges) and be verified through association. Pressing this may also send them the “Accept” email and remove them from this list. The deny button may also remove them from the list, as well as send them the rejection email. They may not be given access.

FIG. 102 may be visible for the encrypted messaging system staff to view and edit, as shown in 10201. When users are unable to verify either themselves or the place they are creating, we may need to do that in-house. When hitting accepts, the user then may receive an email stating his verification and may have full access to medroster.com. When rejected, a rejection email may be sent to the user. When either accept/deny is pressed, the user disappears from this list.

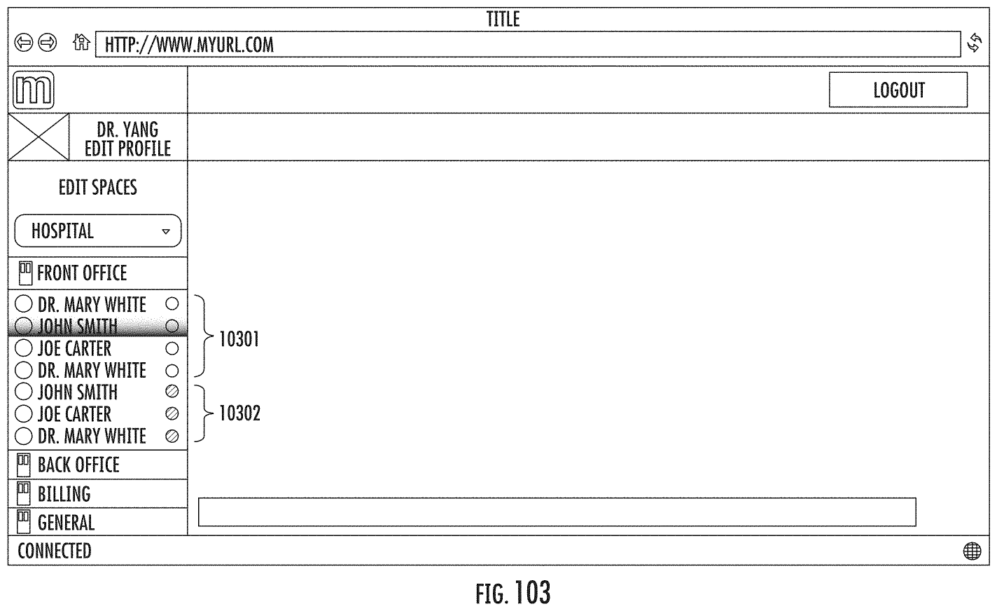

The encrypted messaging system may include online/offline status capability to allow users on the web and mobile (later, after history or contacts are in) to see what users are online and offline. FIG. 103 shows a screen with the online/offline status capability. FIG. 103 includes:

10301. Green Dot (or empty dot) indicates User is online

10302. Grey dot (or hatched dot) indicates user is offline.

Online users should be grouped together on top. Online users should be in alphabetical order. Offline users should be grouped together on bottom. Offline users should be in alphabetical order.

If a patient type user tries to contact someone from a space and no staff is online, that user should receive a message saying no one is online and that their message may be returned when someone is online.

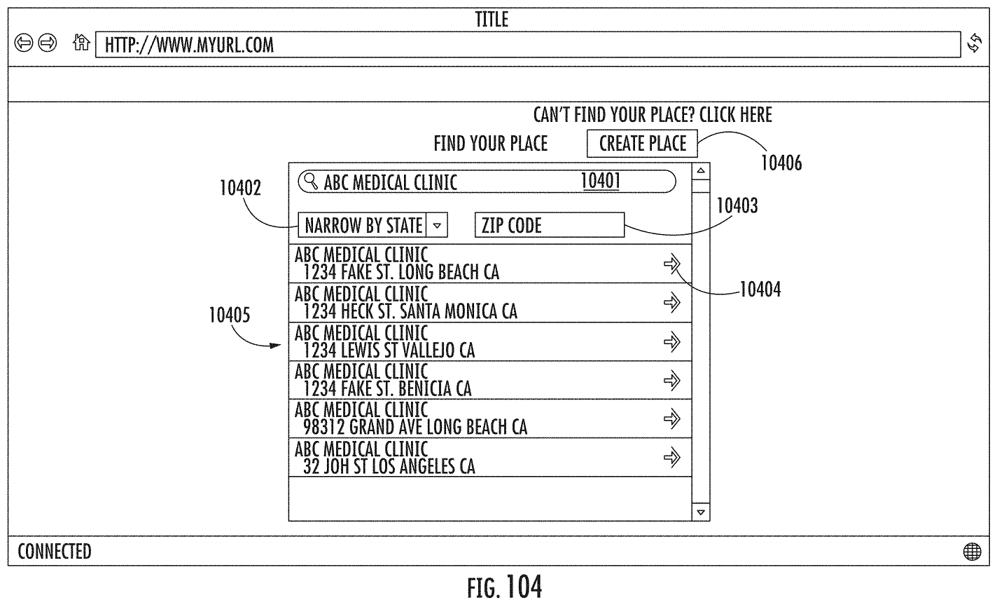

FIGS. 104-111 show a series of screens on how to add places for an encrypted messaging system. The encrypted messaging system may refer to any medical business or establishment as a place.

The encrypted messaging system may allow multiple methods to create a place. For example, creating a new place may occur during a signup process. If a user wishes to activate/create a new place, the user would need to sign up under a separate email address (e.g., use an email that does not exist in the encrypted messaging system). To solve this problem, the encrypted messaging system allows a user the ability to activate/create a new place while they are logged in (e.g., logged into the encrypted messaging system with an email already stored in the encrypted messaging system).

FIG. 104 shows a screen to make a search for a place. Similar to the signup process, a user would need to first search for the place they are attempting to create/activate. They may be directed to find their place, and if they cannot, they may have the option to create their own. FIG. 104 includes:

10401. Enter name or address of place to search

10402. Narrow by state

10403. Narrow by zip code

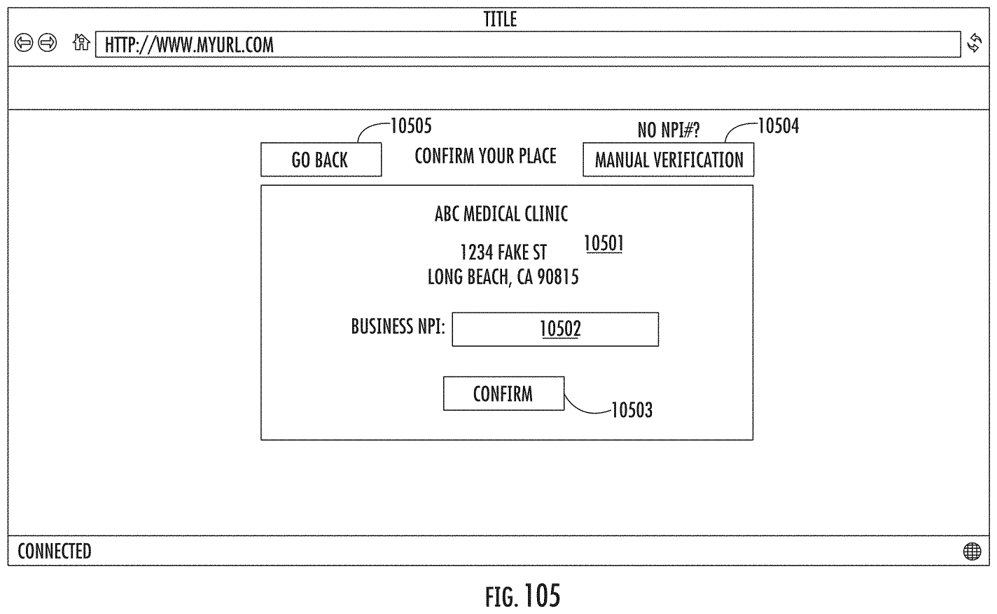

10404. Selecting a specific place may take the user to confirm page (FIG. 105)

10405. List of places that match search field

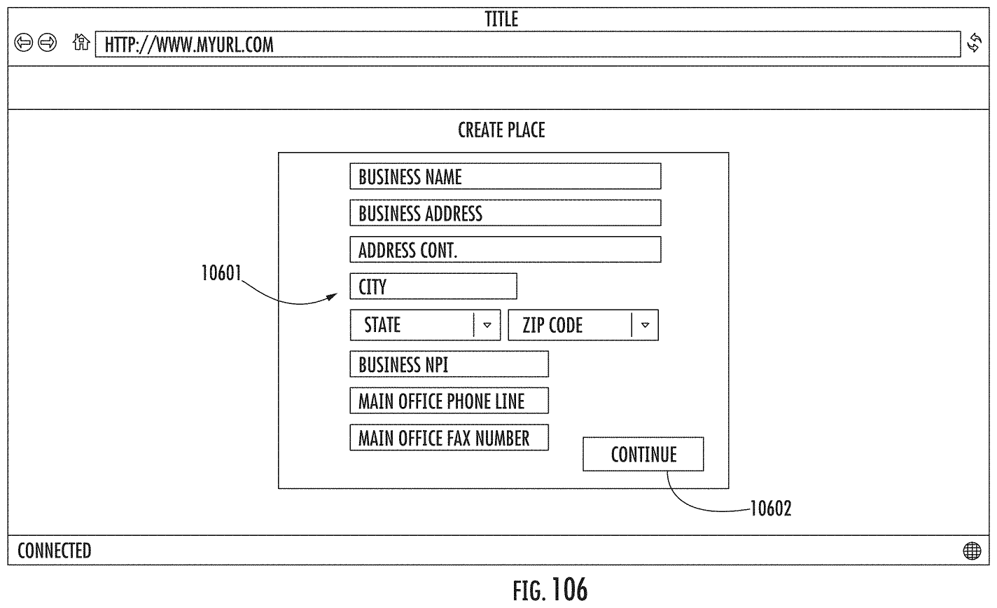

10406. If place is not in database, user can create their own and wait for administrative approval (FIG. 106).

FIG. 105 shows a screen to confirm a place. For example, the screen may be presented to a user after they have selected a place from FIG. 104. FIG. 105 includes:

10501. Display of selected place

10502. User may need to enter National Provider Identifier (NPI) of place to confirm

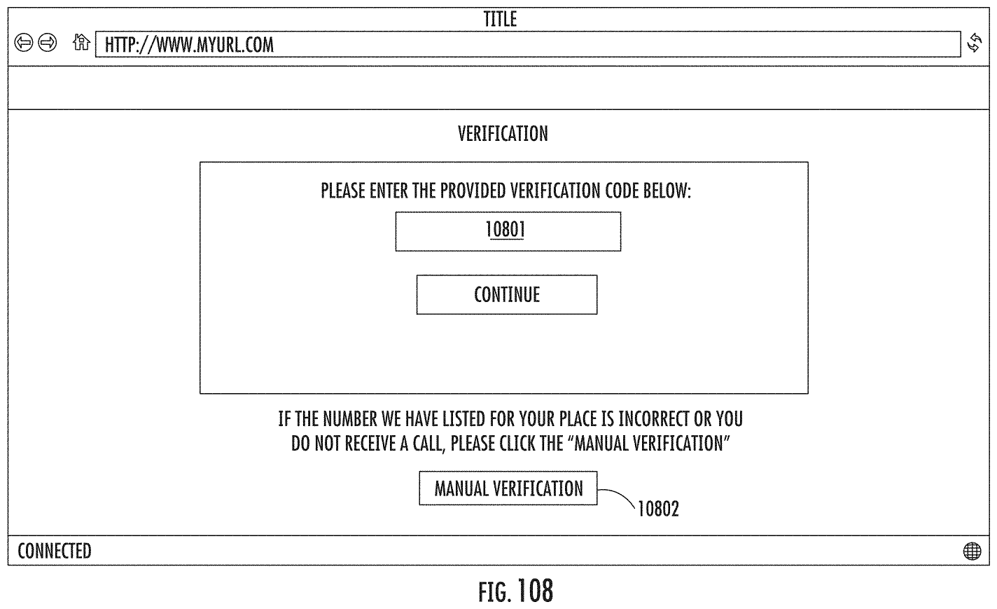

10503. Clicking confirm may check NPI against database info. If correct, the user may be shown FIG. 108 for phone confirmation

10504. If user has no NPI, they may be shown FIG. 108 (manual verification)

10505. User can go back if wrong place is selected (goes back to place search FIG. 104).

FIG. 106 shows a screen to create a place. Users selecting “create a place” on FIG. 104 may be directed to this screen. FIG. 106 includes:

10601. User enters info into all fields

10602. If all info is collected, user may continue to manual verification screen (FIG. 108).

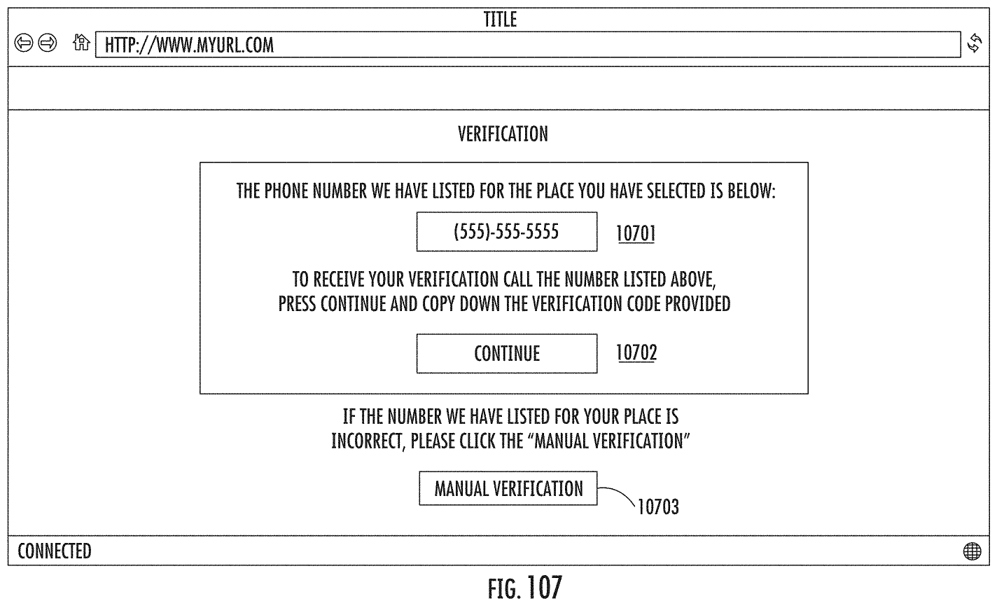

FIG. 107 shows a screen to verify a place. Using the main phone number the encrypted messaging system has in a database, once the user selects “continue” the encrypted messaging system may call the user and provide a verification code. FIG. 107 includes:

10701. The users main phone number from the database

10702. Pressing “continue” may prompt the system to call the phone number in #1 and move to the next screen (FIG. 108)

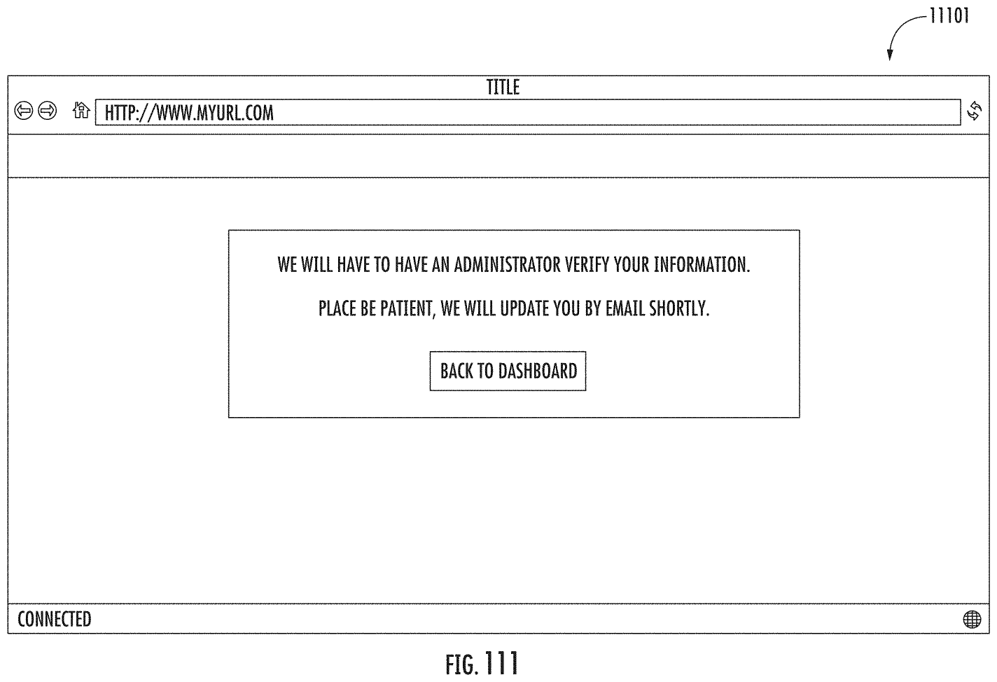

10703. Pressing “manual verification” may take the user to screen shown in FIG. 111 and send the user information to the “admin verification” screen.

FIG. 108 shows a screen to verify a phone. After the user prompts the system to call the database provided phone number, the user may receive a call to that number. The encrypted messaging system may provide the user a code to enter into the text field. FIG. 108 includes:

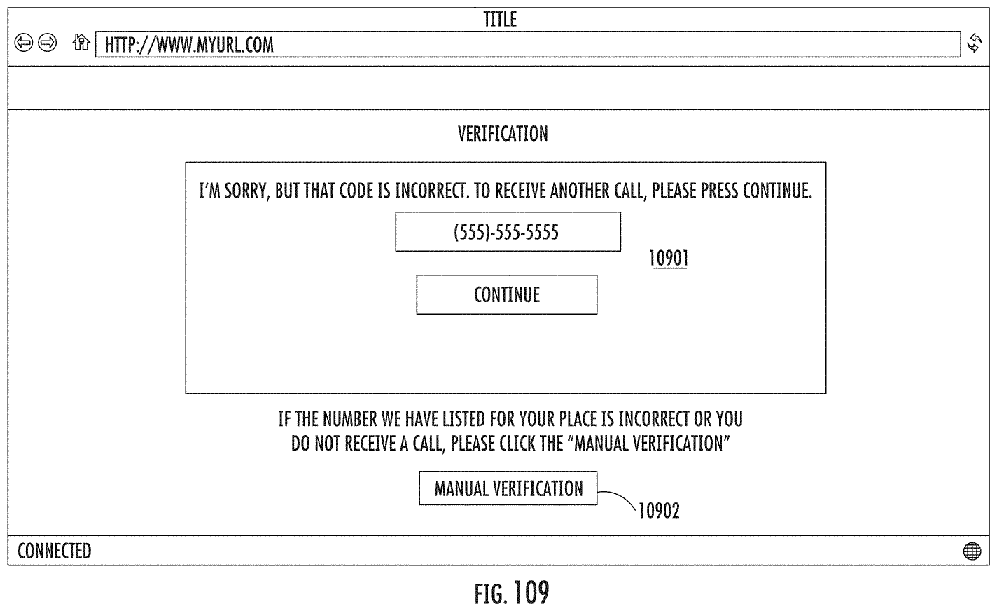

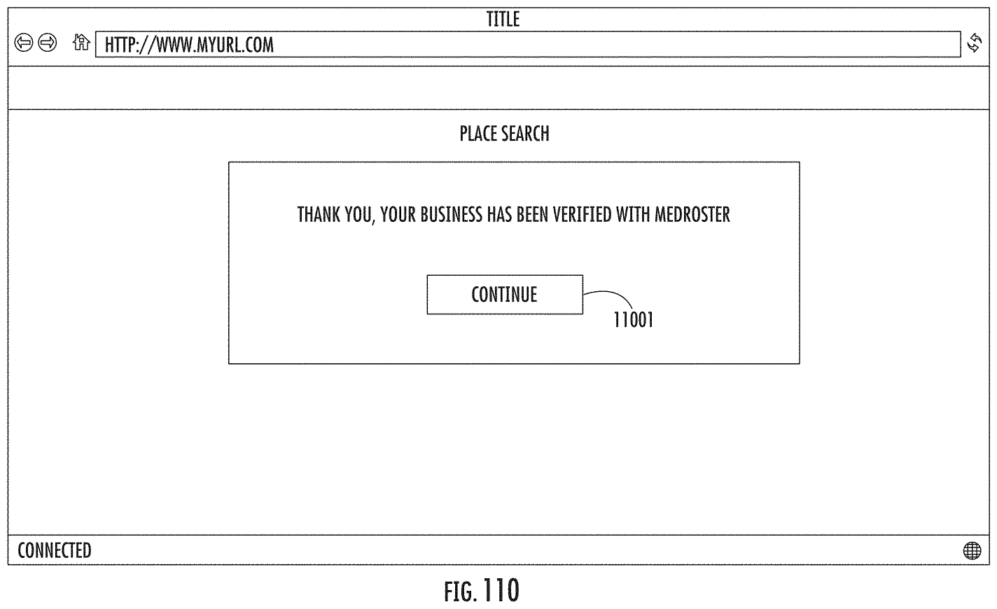

10801. Text field for verification code. Pressing continue with the correct verification code may take the user to the screen shown in FIG. 110. An incorrect code may take the user to the screen shown in FIG. 109.

10802. Pressing “manual verification” may take the user's data to the Admin verification screen.

FIG. 109 shows a screen when there is a phone verification error. If the user enters an incorrect code, they may view this screen. FIG. 109 includes:

10901. The user has the option to either receive another call or manually verify their information. This cycle may continue until the user enters the correct verification code. Pressing continue may send the user to 107 to resend the call.

10902. Manual verification.

FIG. 110 shows a screen when phone verification completes successfully. Pressing continue 11001 may take them to the dashboard.

FIG. 111 shows a screen 11101 to perform manual verification. For example, if a user attempts to create/activate a place but cannot verify the NPI or the main phone number, the user may have to be verified by an encrypted messaging system admin. Clicking “back to dashboard” may take them back to an account page. They should always be logged in.

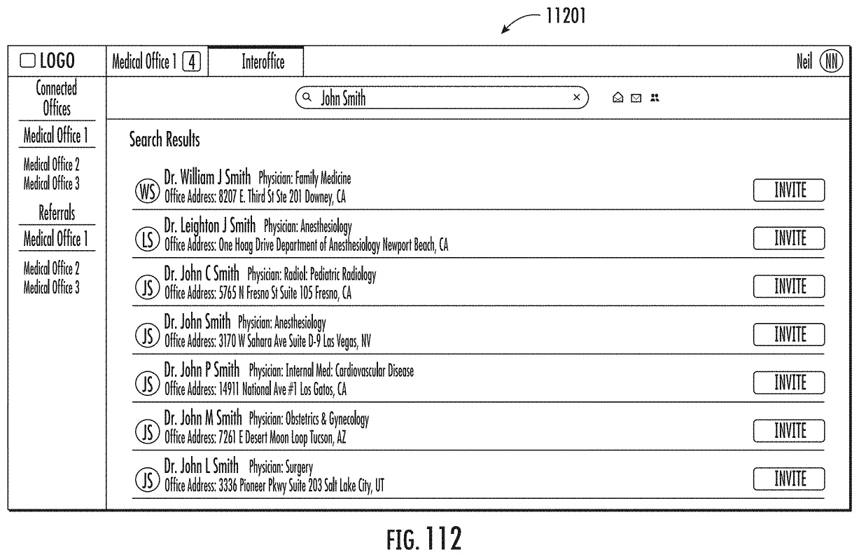

FIG. 112 shows a screen of a search results pages with points of reference, as shown in 11201. Points of reference may include full name, practice area, address, or other relevant user information. The points of reference may be sorted by closest practitioner to the place location first and listing in descending order from closest to furthest (based on geolocation information of the searching user).

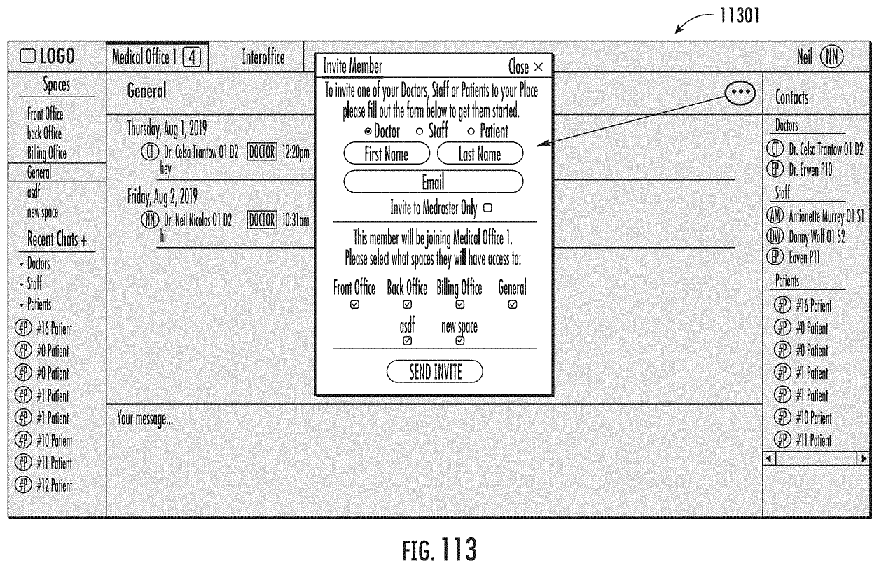

FIG. 113 shows a screen of an invite form, as shown in 11301. For example, this screen shows how to add colleagues to the encrypted messaging system. This screen consolidates invite details on a single screen, to simply user adding users to a Medroster team.

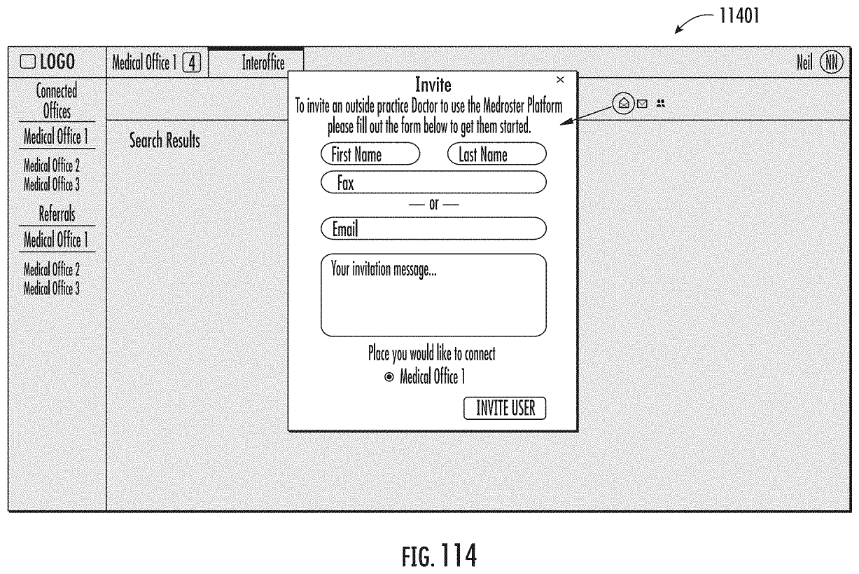

FIG. 114 shows a screen of an invite form, without adding them to an existing medical practice, as shown in 11401. For example, this may be used to invite users to try the Medroster platform and does not add them to your team.

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description may enable others skilled in the art to best utilize and practice the invention in various embodiments and with various modifications as are suited to a particular use. The scope of the invention is defined by the following claims.

The invention claimed is:

1. A method comprising:
fetching, from a key server, a first encryption key for a first conversation from a first client device intended for a first functional unit that corresponds to a second client device;
encrypting the first conversation using the first encryption key to create a first encrypted conversation;
storing a first timestamp and a first conversation identifier that uniquely identifies the first encrypted conversation with the first encryption key;
transmitting the first encrypted conversation to a message server;
when exceeding an expiration period for the first conversation according to the first timestamp, causing to be deleted the first encrypted conversation from the message server;
receiving from the second client device a request to retrieve the first encrypted conversation from the message server;
if the first encrypted conversation has been deleted, responding to the second client device that the first encrypted conversation has been deleted;
if the first encrypted conversation has not been deleted, causing to be determined at the second client device, based on the first conversation identifier, whether the second client device has stored a copy of a first decryption key; and
receiving, based on the second client device not storing a copy of the first decryption key, a request at the key server for the first encryption key.

2. The method of claim 1 wherein the first conversation comprises an interoffice communication.

3. The method of claim 1 wherein the first conversation comprises an intraoffice communication.

4. The method of claim 1 comprising if the key server has not found the first encryption key, returning in response to the fetching the first encryption key a new encryption key generated to be used as the first encryption key.

5. The method of claim 1 comprising:
before the first encrypted conversation is transmitted to the second client device, transmitting the first encrypted conversation to the message server; and
transferring, from the message server, the first conversation to the second client device.

6. The method of claim 5 wherein the message server and the key server are separate servers.

7. The method of claim 5 wherein exceeding the expiration period for the first conversation according to the first timestamp comprises deleting the first encrypted conversation from the second client device.

8. The method of claim 1 wherein the medical device corresponds to the second client device and a third client device.

9. The method of claim 1 wherein the second client device comprises an identity verified user.

10. The method of claim 9 wherein the first client device comprises another identity verified user.

11. The method of claim 10 wherein the identity verified user or the other identity verified user comprises a user verified through one or more identity verification methods including a unique business phone or fax number verification.

12. The method of claim 10 wherein the identity verified user or the other identity verified user comprises a user verified through one or more identity verification methods including a mobile phone verification and access to conversations depends on a user type of the user.

13. The method of claim 1 wherein the first client device comprises a medical office staff member user.

14. The method of claim 1 wherein the second client device comprises a patient user.

15. The method of claim 1 wherein the first timestamp corresponds to a time when the first conversation was sent from the first client device to the second client device.

16. The method of claim 1 wherein the first functional unit corresponds to a medical office and the medical office further corresponds to a second functional unit that is different than the first functional unit.

17. The method of claim 1 wherein the first encryption key may decrypt one or more messages in the first encrypted conversation.

18. The method of claim 1 wherein the first encryption key cannot decrypt a second encrypted conversation from the first client device intended for the first functional unit that corresponds to the second client device.

19. A method for a secured messaging system comprising:
receiving from a patient user's device a request to secure a first message to be sent to a first functional unit of a medical office;
in response to the patient user's request, selecting a first encryption key;
creating, based on the first message and the first encryption key, a secured first message;
causing storing the secured first message and a unique identifier for the secured first message;
determining, based on the medical office, medical office users of the secured messaging system associated with the medical office;
determining, based on the medical office users and the first functional unit, first and second users of the secured messaging system associated with the first functional unit and the medical office;
transmitting the secured first message to devices associated with the first and second users; and
causing decrypting, based on the unique identifier of the secured first message and a first decryption key being stored on the first user's device before the first user attempting to open the secured first message, the first secured message on the first user's device.

20. The method of claim 19 comprising:
causing transmitting, based on the unique identifier of the secured first message and that the first decryption key is not stored on the second user's device before the first user attempting to open the secured first message, the first decryption key to the second user's device; and
decrypting the first secured message on the second user's device.

21. The method of claim 19 wherein transmitting the first decryption key and transmitting the secured first message occurs during different transmissions.

22. The method of claim 19 wherein the first user comprises a staff role user and the second user comprises a doctor role user.

23. The method of claim 19 comprising:
receiving from the first user's device a request to secure a second message to be sent to the patient user;
in response to the first user's request, selecting a second encryption key;
creating, based on the second message and the second encryption key, a secured second message;
causing storing the secured second message and a unique identifier for the secured second message;
transmitting the secured second message to the patient user's device; and
causing decrypting, based on the unique identifier of the secured second message and that a second decryption key is stored on the patient user's device before transmitting the second secured message, the second secured message on the patient user's device.

24. The method of claim 23 wherein the secured second message includes an indication that the first user is responding to the patient user on behalf of the second user.

25. The method of claim 19 comprising:
receiving from the first user's device a request to secure a second message to be sent to the patient user;
in response to the first user's request, selecting a second encryption key;
creating, based on the second message and the second encryption key, a secured second message;
causing storing the secured second message and a unique identifier for the secured second message;
transmitting the secured second message to the patient user's device;
causing transmitting, based on the unique identifier of the secured second message and that the second decryption key is not stored on the patient user's device before transmitting the second secured message, the second decryption key to the patient user's device; and
decrypting the second secured message on the patient user's device.
